(19) United States
(12) Patent Application Publication
(10) Pub. No.: US 2024/0217989 A1
(43) Pub. Date: Jul. 4, 2024

(54) Heteroaryl Fluoroalkenes As DGK Inhibitors

(71) Applicant: Incyte Corporation, Wilmington, DE (US)

(72) Inventors: Ming Xiang, Wilmington, DE (US); Joshua Hummel, Hockessin, DE (US); Shaoqun Qian, Claymont, DE (US); Shicheng Shi, Princeton, NJ (US); Xiaozhao Wang, Moorestown, NJ (US)

(21) Appl. No.: 18/512,514

(22) Filed: Nov. 17, 2023

Related U.S. Application Data
(60) Provisional application No. 63/426,595, filed on Nov. 18, 2022.

(51) Int. Cl. C07D 498/10; C07D 401/04; C07D 401/14; C07D 403/14; C07D 417/14; C07D 471/04; C07D 471/10

(52) U.S. Cl. CPC C07D 498/10; C07D 401/04; C07D 401/14; C07D 403/14; C07D 417/14; C07D 471/04; C07D 471/10

(57) ABSTRACT

The present application provides heteroaryl fluoroalkene compounds that modulate the activity of diacylglycerol kinase (DGK), which are useful in the treatment of various diseases, including cancer.

### TECHNICAL FIELD

The present invention provides heteroaryl fluoroalkene compounds that modulate the activity of diacylglycerol kinase (DGK) and are useful in the treatment of diseases related to diacylglycerol kinase, including cancer.

### BACKGROUND

Diacylglycerol kinases (DGKS) are a family of enzymes that regulate many biological processes, including cellular proliferation, migration, immunity and pathogenesis of diseases such as cancer. In mammalian systems, there are ten DGK family members classified into five subtypes based on shared common domains (Sakane F. et al., Int. J. Mol. Sci., 2020. 21: p6794-6829). The diverse and specific cellular function of individual DGK isoforms is regulated through their tissue restricted expression, localization within cells and interactions with regulatory proteins (Joshi, R. P. and Koretzky, G. A., Int. J. Mol. Sci., 2013. 14: p6649-6673).

In T lymphocytes, DGKα and ζ are the dominant DGK isoforms expressed (Krishna, S. and Zhong, X.-P., Front Immunol., 2013. 4:178). Specifically, in response to T cell receptor (TCR) activation, phospholipase Cγ1 (PLCγ1) hydrolyzes membrane phospholipid PIP2 to produce diacylglycerol (DAG) (Krishna, S. and Zhong, X.-P., Front Immunol., 2013. 4:178; Riese, M. J. et al., Front Cell Dev Biol., 2016. 4:108). In turn, DAG functions as a second messenger to recruit RasGRP1 and PKCθ to the cell membrane and thereby initiates multiple downstream signaling events resulting in T cell activation. To prevent hyperactivation of T cells, DGKα and ζ tightly regulate the levels of intracellular DAG by phosphorylating DAG to produce phosphatidic acid (PA). Both mouse and human cell line genetic studies support the important regulatory role of DGKα and (in T cell activation. Knockout or depletion of DGKα and ζ has been reported to enhance T cell activation, cytokine production and proliferation. Furthermore, knockout of both DGKα and ζ show even greater T-cell activation over individual knockouts, indicating a non-redundant role of these two isoforms (Riese, M. J. et al., Cancer Res., 2013. 73:p3566-3577; Jung, I.-Y. et al., Cancer Res., 2018. 78: p4692-4703). Thus, DGKα and ζ, by regulating cellular DAG levels link lipid metabolism and intracellular signaling cascades and function as key regulators of T cell activation.

Cytotoxic T lymphocytes (CTLs) are a major component of the adaptive immune system that recognize and kill cells with bacterial or viral infections, or cells displaying abnormal proteins, such as tumor antigens. However, cancer cells can evolve to utilize multiple mechanisms that mimic peripheral immune tolerance to avoid immune surveillance and killing by CTLs. Such mechanisms include downregulation of antigen presentation, suppression of T cell function through increased expression of inhibitory molecules, as well as increased production of immunosuppressive proteins in the tumor microenvironment (Speiser, D. E. et al., Nat. Rev. Immunol., 2016. 16: p. 599-611, Gonzalez H. et al., Genes & Dev., 2018. 32:p1267-1284). Immune checkpoint therapy (ICT) by blocking inhibitory molecules such as PD(L)-1 and CTLA4, can restore T cell activity and have been clinically useful in treating many different types of cancers. However, only subsets of patients respond to ICT due to primary or acquired resistance (Sharma, P. et al., Cell. 2017. 168: p707-723). Thus, despite the significant recent clinical successes of immunotherapies to treat cancer, resistance remains a challenge (Sharma, P., et al., Cancer Discov., 2021. 11: p838-857).

Overexpression of DGKα and ζ has been observed in tumor infiltrating lymphocytes (TILs) from human tumors and proposed to suppress T cell function. Importantly, significant immune-mediated antitumor activity has been shown in DGKα and DGKζ deficient mouse models (Merida, I. et al., Adv. Biol. Regul., 2017. 63:p22-31, Prinz, P. U. et al., J. Immunol., 2012. 188:p5990-6000). Furthermore, DGKα and DGKζ deficient T cells are resistant to several immunosuppressive factors within the tumor microenvironment such as TGFβ, PGE2 and adenosine, and to other T cell inhibitory pathways such as PD(L)-1 mediated immune suppression (Riese, M. J. et al., Cancer Res., 2013. 73:p3566-77; Jung, I.-Y. et al. (2018) Cancer Res., 2018. 78:p4692-4703; Arranz-Nicolas, J. et al., Cancer Immunol. Immunother., 2018. 67:p965-980; Riese, M. J. et al., Front. Cell Dev. Biol., 2016. 4:108). Thus DGKα and DGKζ are attractive targets as immunotherapies alone or in combination with current ICT therapies such as PD(L)-1 and CTLA4. By targeting T cell lipid metabolism, DGKα and DGKζ inhibition can potentially restore antitumor immunity in subsets of patient who have primary or acquired immune resistance and are consequently refractory to current ICTs. In addition to its function in T lymphocytes, DGKα and DGKζ, by regulating DAG level in cancer cells, have also been reported to directly contribute to cancer proliferation, migration, invasion and survival. Thus, DGK inhibition may have direct antitumor effect by interfering with tumor intrinsic oncogenic survival pathways (Cooke, M. and Kaznietz, M. G., Sci. Signal., 2022. 15:eabo0264).

Compounds in this application may have selective activities towards one or both DGKα and DGKζ. These DGK inhibitors alone or in combination with other therapeutic agent(s) can be used in treatment of cancer.

### SUMMARY

The present invention relates to, inter alia, compounds of Formula I:

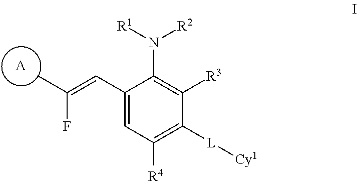

or pharmaceutically acceptable salts thereof, wherein constituent members are defined herein.

The present invention further provides pharmaceutical compositions comprising a compound of Formula I, or a pharmaceutically acceptable salt thereof, and a pharmaceutically acceptable carrier.

The present invention further provides methods of inhibiting an activity of diacylglycerol kinase (DGK), comprising contacting the kinase with a compound of Formula I, or a pharmaceutically acceptable salt thereof.

The present invention further provides methods of treating a disease or a disorder associated with expression or activity of a diacylglycerol kinase (DGK) in a patient by administering to a patient a therapeutically effective amount of a compound of Formula I, or a pharmaceutically acceptable salt thereof.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DETAILED DESCRIPTION

The present application provides a compound of Formula I:

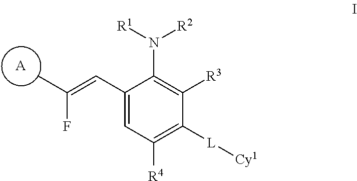

or a pharmaceutically acceptable salt thereof, wherein:
Ring A is:

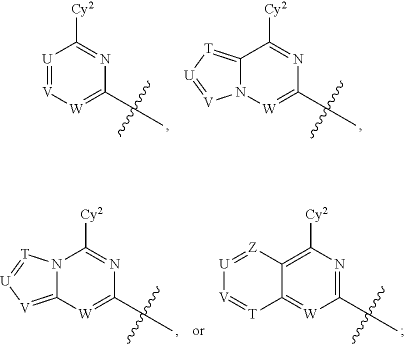

T is CR⁸ or N;
U is CR⁵ or N;
V is CR⁶ or N;
W is CR⁷ or N;
Z is CH or N;
wherein at least one of T, U, V, or W is not N;
L is a bond, O, or NR¹⁰.
Cy¹ is selected from C₆₋₁₀ aryl, 5-10 membered heteroaryl, 3-10 membered cycloalkyl, and 4-10 membered heterocycloalkyl, wherein the C₆₋₁₀ aryl, 5-10 membered heteroaryl, 3-10 membered cycloalkyl, and 4-10 membered heterocycloalkyl are each optionally substituted with 1, 2, 3, 4, 5, 6, 7, or 8 independently selected R¹¹ substituents;
each R¹¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a111}, SR^{a111}, NHOR^{a111}, C(O)R^{b111}, C(O)NR^{c111}R^{d111}, C(O)NR^{c111}(OR^{a111}), C(O)OR^{a111}, OC(O)R^{b111}, OC(O)NR^{c111}R^{d111}, NR^{c111}R^{d111}, NR^{c111}R^{c111}R^{d111}, NR^{c111}C(O)R^{b111}, NR^{c111}C(O)OR^{a111}, NR^{c111}C(O)NR^{c111}R^{d111}, C(═NR^{e111})R^{b111}, C(═NR^{e111})NR^{c111}R^{d111}, NR^{c111}C(═NR^{e111})NR^{c111}R^{d111}, NR^{e111}C(═NR^{e111})R^{b111}, NR^{c111}S(O)R^{b111}, NR^{c111}S(O)NR^{c111}R^{d111}, NR^{c111}S(O)₂R^{b111}, NR^{c111}S(O)(═NR^{e111})R^{b111}, NR^{c111}S(O)₂NR^{c111}R^{d111}, S(O)R^{b111}, S(O)NR^{c111}R^{d111}, S(O)₂R^{b111}, S(O)₂NR^{c111}R^{d111}, OS(O)(═NR^{e111})R^{b111}, and OS(O)₂R^{b111}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl- of R¹¹ are each optionally substituted with 1, 2, 3, 4, 5, 6, 7, or 8 independently selected R^{11A} substituents;
each R^{a111}, R^{c111}, and R^{d111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a111}, R^{c111} and R^{d11} are each optionally substituted with 1, 2, 3, 4, 5, 6, 7, or 8 independently selected R^{11A} substituents;
or, any R^{c111} and R^{d111} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{11A} substituents;
each R^{b111} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b111} are each optionally substituted with 1, 2, 3, 4, 5, 6, 7, or 8 independently selected R^{11A} substituents;
each R^{e111} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{11A} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a112}, C(O)NR^{c112}R^{d112}, C(O)OR^{a112} NR^{c112}R^{d112} S(O)NR^{c112}R^{a112}, S(O)₂R^{b112}, S(O)₂NR^{c112}R^{a112}, and OS(O)₂R^{b112}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{11A} are each optionally substituted with 1, 2, 3, or 4 independently selected R^{M} substituents;
each R^{a112}, R^{c112}, and R^{d112} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a112}, R^{c112} and R^{d112} are each optionally substituted with 1, 2, 3, or 4 independently selected R^{M} substituents;
or, any R^{c112} and R^{d112} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b112} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b112} are each optionally substituted with 1, 2, 3, or 4 independently selected R^{M} substituents;
Cy² is 4-pyridazinyl, 5 membered heteroaryl, or 4-8 membered heterocycloalkyl, wherein the 4-pyridazinyl, 5 membered heteroaryl, and 4-8 membered heterocycloalkyl are each optionally substituted with 1, 2, 3, or 4 independently selected R¹² substituents;
R¹² is selected from halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OR^{a121}, CN, C(O)OH, C(O)NHR^{a121}, and NR^{a121}R^{a121}, wherein the C₁₋₆ alkyl is optionally substituted with OH, CN, and NH₂;
each R^{a121} is independently selected from H, C₁₋₆ alkyl, C₁₋₆ alkoxy, C₁₋₆ haloalkyl, C₁₋₆haloalkoxy, OH, NH₂, and NHC₁₋₆ alkyl, wherein the C₁₋₆ alkyl is optionally substituted with OH, CN, and NH₂;
R¹ is selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, C(═NR^{e1})R^{b1}, C(═NR^{e1})NR^{c1}R^{d1}, C(═NOR^{a1})R^{b1}, C(═NOR^{a1})OR^{a1}, S(O)R^{b1}, S(O)NR^{c1}R^{d1}, S(O)₂R^{b1}, S(O)₂NR^{c1}R^{d1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹ are each optionally substituted with 1, 2, 3, 4, 5, 6, 7, or 8 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a1}, R^{b1} and R^{d1} are each optionally substituted with 1, 2, 3, 4, 5, 6, 7, or 8 independently selected R^{1A} substituents;
or, any R^{c1} and R^{d1} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{1A} substituents;
each R^{b1} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b1} are each optionally substituted with 1, 2, 3, 4, 5, 6, 7, or 8 independently selected R^{1A} substituents;
each R^{e1} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, 4, 5, 6, 7, or 8 independently selected R¹ substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a11}, R^{c11} and R^{d11} are each optionally substituted with 1, 2, 3, 4, 5, 6, 7, or 8 independently selected R¹ substituents;
or, any R^{c11} and R^{d11} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{1B} substituents;
each R^{b11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b11} are each optionally substituted with 1, 2, 3, 4, 5, 6, 7, or 8 independently selected R¹ substituents;
each R^{e11} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1B} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a12}, C(O)NR^{c12}R^{d12}, C(O)OR^{a12}, NR^{c12}R^{a12}, S(O)NR^{c12}R^{d12}, S(O)₂R^{b12}, S(O)₂NR^{c12}R^{d12}, and OS(O)₂R^{b12}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{B} are each optionally substituted with 1, 2, 3, or 4 independently selected R^{M} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a12}, R^{c2} and R^{d12} are each optionally substituted with 1, 2, 3, or 4 independently selected R^{M} substituents;
or, any R^{c2} and R^{d12} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b12} are each optionally substituted with 1, 2, 3, or 4 independently selected R^{M} substituents;
R² is selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, C(═NR^{e2})R^{b2}, C(═NR^{e2})NR^{c2}R^{d2}, C(═NOR^{a2})R^{b2}, C(═NOR^{a2})OR^{a2}, S(O)R^{b2}, S(O)NR^{c2}R^{d2}, S(O)₂R^{b2}, and S(O)₂NR^{c2}R^{d2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21} NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2B} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a22}, C(O)NR^{c22}R^{d22}, C(O)OR^{a22}, NR^{c22}R^{d22}, S(O)NR^{c22}R^{d22}, S(O)₂R^{b22}, S(O)₂NR^{c22}R^{d22}, and OS(O)₂R^{b22}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{2B} are each optionally substituted with 1, 2, 3, or 4 independently selected R^{M} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a22}, R^{c22} and R^{d22} are each optionally substituted with 1, 2, 3, or 4 independently selected R^{M} substituents;
or, any R^{c22} and R^{d22} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b22} are each optionally substituted with 1, 2, 3, or 4 independently selected R^{M} substituents;
or, R¹ and R², together with the N atom to which they are attached, form a 4-12 membered heterocycloalkyl, wherein the 4-12 membered heterocycloalkyl is optionally substituted with 1, 2, 3, 4, 5, 6, 7, or 8 independently selected R¹³ substituents;
each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a131}, SR^{a131}, NHOR^{a131}, C(O)R^{b131}, C(O)NR^{c131}R^{d131}, C(O)NR^{c131}(OR^{a131}), C(O)OR^{a131}, OC(O)R^{b131}, OC(O)NR^{c131}R^{d131}, NR^{c131}R^{d131}, NR^{c131}NR^{c131}R^{d131}, NR^{c131}C(O)R^{b131}, NR^{c131}C(O)OR^{a131}, NR^{c131}C(O)NR^{c131}R^{d131}, C(═NR^{e131})R^{b131}, C(═NR^{e131})NR^{c131}R^{d131}, C(═NOR^{a131})R^{b131}, C(═NOR^{a131})OR^{a131}, NR^{c131}C(═NR^{e131})NR^{c131}R^{d131}, NR^{c131}C(═NR^{e131})R^{b131}, NR^{c131}S(O)R^{b131}, NR^{c131}S(O)NR^{c131}R^{d131}, NR^{c131}S(O)₂R^{b131}, NR^{b131}S(O)(═NR^{e131})R^{b131}, NR^{c131}S(O)₂NR^{c131}R^{d131}, S(O)R^{b131}, S(O)NR^{c131}R^{d131}, S(O)₂R^{b131}, S(O)₂NR^{c131}R^{d131}, OS(O)(═NR^{e131})R^{b131}, and OS(O)₂R^{b131}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1, 2, 3, 4, 5, 6, 7, or 8 independently selected R^{13A} substituents;
each R^{a131}, R^{c131}, and R^{d131} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a131}, R^{c131} and R^{d131} are each optionally substituted with 1, 2, 3, 4, 5, 6, 7, or 8 independently selected R^{13A} substituents;
or, any R^{c131} and R^{d131} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{13A} substituents;
each R^{b131} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b131} are each optionally substituted with 1, 2, 3, 4, 5, 6, 7, or 8 independently selected R^{13A} substituents;
each R^{e131} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{13A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a132}, SR^{a132}, NHOR^{a132}, C(O)R^{b132}, C(O)NR^{c132}R^{d132}, C(O)NR^{c132}(OR^{a132}), C(O)OR^{a132}, OC(O)R^{b132}, OC(O)NR^{c132}R^{d132}, NR^{c132}R^{d132}, NR^{c132}NR^{c132}R^{d132} NR^{c132}C(O)R^{b132}, NR^{c132}C(O)OR^{a132}, NR^{c132}C(O)NR^{c132}R^{d132}, C(═NR^{e132})R^{b132}, C(═NR^{e132})NR^{c132}R^{d132}, NR^{c132}C(═NR^{e132})NR^{c132}R^{d132}, NR^{c132}C(═NR^{e132})R^{b132}, NR^{c132}S(O)R^{b132}, NR^{c132}S(O)NR^{c132}R^{d132}, NR^{c132}S(O)₂R^{b132}, NR^{c132}S(O)(═NR^{e132})R^{b132}, NR^{c132}S(O)₂NR^{c132}R^{d132}, S(O)R^{b132}, S(O)NR^{c132}R^{d132}, S(O)₂R^{b132}, S(O)₂NR^{c132}R^{d132}, OS(O)(═NR^{e132})R^{b132}, and OS(O)₂R^{b132}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{13A} are each optionally substituted with 1, 2, 3, 4, 5, 6, 7, or 8 independently selected R^{13B} substituents;
each R^{a132}, R^{c132}, and R^{d132} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a132}, R^{c132} and R^{d132} are each optionally substituted with 1, 2, 3, 4, 5, 6, 7, or 8 independently selected R^{13B} substituents;
or, any R^{c132} and R^{d132} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{13B} substituents;
each R^{b132} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b132} are each optionally substituted with 1, 2, 3, 4, 5, 6, 7, or 8 independently selected R^{13B} substituents;
each R^{e132} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{13B} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a133}, C(O)NR^{c133}R^{d133}, C(O)OR^{a133}, NR^{c133}R^{d133}, S(O)NR^{c133}R^{d133}, S(O)₂R^{b133}, S(O)₂NR^{c133}R^{d133}, and OS(O)₂R^{b133}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{13B} are each optionally substituted with 1, 2, 3, or 4 independently selected R^{M} substituents;
each R^{a133}, R^{c133}, and R^{d133} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a133}, R^{c133} and R^{d133} are each optionally substituted with 1, 2, 3, or 4 independently selected R^{M} substituents;
or, any R^{c133} and R^{d133} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b133} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b133} are each optionally substituted with 1, 2, 3, or 4 independently selected R^{M} substituents;
R³ is selected from H, halo, C₁₋₆ alkyl and C₁₋₆ haloalkyl;
R⁴ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₄ cycloalkyl, OH, CN, C(O)OH, C(O)NH₂, and NH₂; wherein the C₁₋₆ alkyl is optionally substituted with OH, CN, and NH₂;
R⁵ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a51}, and NHR^{a52}, wherein the C₁₋₆ alkyl is optionally substituted with OH, CN, and NH₂;
each R^{a51} is independently selected from H, C₁₋₆ alkyl, C₁₋₆ alkoxy, C₁₋₆ haloalkyl, C₁₋₆haloalkoxy, OH, NH₂, and NHC₁₋₆ alkyl, wherein the C₁₋₆ alkyl is optionally substituted with OH, CN, and NH₂;
each R^{a52} is independently selected from C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R⁶ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, and C(O)NH₂; wherein the C₁₋₆ alkyl is optionally substituted with OH, CN, and NH₂;
R⁷ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a71}, and NHR^{a72}, wherein the C₁₋₆ alkyl is optionally substituted with OH, CN, and NH₂;
each R^{a71} is independently selected from H, C₁₋₆ alkyl, C₁₋₆ alkoxy, C₁₋₆ haloalkyl, C₁₋₆haloalkoxy, OH, NH₂, and NHC₁₋₆ alkyl, wherein the C₁₋₆ alkyl is optionally substituted with OH, CN, and NH₂;
each R^{a72} is independently selected from C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R⁸ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a81}, and NHR^{a82}, wherein the C₁₋₆ alkyl is optionally substituted with OH, CN, and NH₂;
each R^{a81} is independently selected from H, C₁₋₆ alkyl, C₁₋₆ alkoxy, C₁₋₆ haloalkyl, C₁₋₆haloalkoxy, OH, NH₂, and NHC₁₋₆ alkyl, wherein the C₁₋₆ alkyl is optionally substituted with OH, CN, and NH₂;
each R^{a82} is independently selected from C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R¹⁰ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NH₂, and NH₂; wherein the C₁₋₆ alkyl, C₂₋₆ alkenyl, and C₂₋₆ alkynyl of R¹⁰ are each optionally substituted with 1, 2, 3, or 4 independently selected R^{M} substituents; and
each R^{M} is independently selected from H, OH, halo, oxo, CN, C(O)OH, NH₂, NO₂, SF₅, C₁₋₆ alkyl, C₁₋₆ alkoxy, C₁₋₆haloalkoxy, C₁₋₆haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-.

In some embodiments, at least two of T, U, V, or W is not N.

In some embodiments, Ring A is:

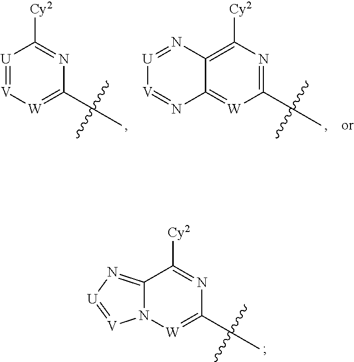

and at least one of U, V, or W is not N.

In some embodiments, Ring A is W %

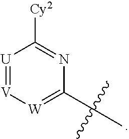

In some embodiments, Ring A is

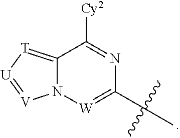

In some embodiments, Ring A is

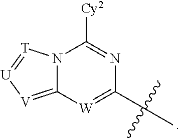

In some embodiments, Ring A is

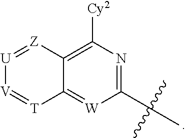

In some embodiments, Ring A is

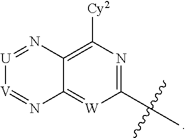

In some embodiments, Ring A is

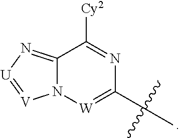

In some embodiments, T is CR⁸.

In some embodiments, R⁸ is selected from H and C₁₋₆ alkyl.

In some embodiments, R⁸ is H.

In some embodiments, T is CH or N.

In some embodiments, T is N.

In some embodiments, U is CR⁵.

In some embodiments, R⁵ is selected from H and C₁₋₆ alkyl.

In some embodiments, R⁵ is H.

In some embodiments, U is CH or N.

In some embodiments, U is N.

In some embodiments, V is CR⁶.

In some embodiments, R⁶ is selected from H and C₁₋₆ alkyl.

In some embodiments, R⁶ is H.

In some embodiments, V is CH.

In some embodiments, V is CH or N.

In some embodiments, V is N.

In some embodiments, W is CR⁷.

In some embodiments, R⁷ is selected from H, halo, C₁₋₆ alkyl, C₁₋₆ alkoxy, C₁₋₆ haloalkyl, C₁₋₆haloalkoxy, C₂₋₆ alkenyl, C₂₋₆ alkynyl, and CN.

In some embodiments, R⁷ is selected from H and CN.

In some embodiments, R⁷ is H.

In some embodiments, R⁷ is CN.

In some embodiments, W is CH, CCN, or N.

In some embodiments, W is N.

In some embodiments, Z is CH.

In some embodiments, Z is N.

In some embodiments, Cy² is 4-pyridazinyl, 5 membered heteroaryl, or 4-8 membered heterocycloalkyl, wherein the 4-pyridazinyl, 5 membered heteroaryl, and 4-8 membered heterocycloalkyl are each optionally substituted with 1 or 2 independently selected R¹² substituents.

In some embodiments, Cy² is 4-pyridazinyl, 5 membered heteroaryl, or 4-6 membered heterocycloalkyl, wherein the 4-pyridazinyl, 5 membered heteroaryl, and 4-6 membered heterocycloalkyl are each optionally substituted with 1, 2, 3, or 4 independently selected R¹² substituents.

In some embodiments, Cy² is 4-pyridazinyl, which is optionally substituted with 1, 2, 3, or 4 independently selected R¹² substituents.

In some embodiments, Cy² is 4-pyridazinyl, which is optionally substituted with 1 or 2 independently selected R¹² substituents.

In some embodiments, Cy² is 5 membered heteroaryl, which is optionally substituted with 1, 2, 3, or 4 independently selected R¹² substituents.

In some embodiments, Cy² is 5 membered heteroaryl, which is optionally substituted with 1 or 2 independently selected R¹² substituents.

In some embodiments, Cy² is 4-8 membered heterocycloalkyl, which is optionally substituted with 1, 2, 3, or 4 independently selected R¹² substituents.

In some embodiments, Cy² is 4-8 membered heterocycloalkyl, which is optionally substituted with 1 or 2 independently selected R¹² substituents.

In some embodiments, Cy² is 4-6 membered heterocycloalkyl, which is optionally substituted with 1, 2, 3, or 4 independently selected R¹² substituents.

In some embodiments, Cy² is 4-6 membered heterocycloalkyl, which is optionally substituted with 1 or 2 independently selected R¹² substituents.

In some embodiments, Cy² is 4-pyridazinyl, isothiazolyl, azetidinyl, or pyrrolidinyl, wherein the 4-pyridazinyl, isothiazolyl, azetidinyl, or pyrrolidinyl are each optionally substituted with 1, 2, 3, or 4 independently selected R¹² substituents.

In some embodiments, Cy² is 4-pyridazinyl, isothiazolyl, azetidinyl, or pyrrolidinyl, wherein the 4-pyridazinyl, isothiazolyl, azetidinyl, or pyrrolidinyl are each optionally substituted with 1 or 2 independently selected R¹² substituents.

In some embodiments, each R¹² is independently selected from halo, C₁₋₆ alkyl, C₁₋₆ alkoxy, C₁₋₆ haloalkyl, C₁₋₆ haloalkoxy, C₂₋₆ alkenyl, and C₂₋₆ alkynyl.

In some embodiments, each R¹² is an independently selected halo.

In some embodiments, each R¹² is fluoro.

In some embodiments, Cy² is 4-pyridizinyl, isothiazolyl, difluoroazetidinyl, or difluoropyrrolidinyl.

In some embodiments, R¹ is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R¹ are each optionally substituted with 1, 2, 3, or 4 independently selected R^{1A} substituents.

In some embodiments, R¹ is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R¹ are each optionally substituted with 1 or 2 independently selected R^{1A} substituents.

In some embodiments, R¹ is C₁₋₆ alkyl, which is optionally substituted with 1, 2, 3, or 4 independently selected R^{1A} substituents.

In some embodiments, R¹ is C₁₋₆ alkyl, which is optionally substituted with 1 or 2 independently selected R^{1A} substituents.

In some embodiments, R¹ is C₁₋₃ alkyl, which is optionally substituted with 1 or 2 independently selected R^{1A} substituents.

In some embodiments, R¹ is methyl or propyl, each of which is optionally substituted with 1 or 2 independently selected R^{1A} substituents.

In some embodiments, each R^{1A} is independently selected from halo, C₁₋₆ alkyl, C₁₋₆ haloalkyl, C₂₋₆ alkenyl, C₂₋₆ alkynyl, and NR^{c11}R^{d11}.

In some embodiments, each R^{a11}, R^{b11}, R^{c11}, and R^{d11} is independently selected from H, C₁₋₆ alkyl, C₁₋₆ haloalkyl, C₂₋₆ alkenyl, and C₂₋₆ alkynyl.

In some embodiments, each R^{a11}, R^{b11}, R^{c11}, and R^{d11} is independently selected from H and C₁₋₆ alkyl.

In some embodiments, each R^{c11} and R^{d11} is independently selected from H and C₁₋₆ alkyl.

In some embodiments, each R^{c11} and R^{d11} is H.

In some embodiments, each R^{1A} is independently selected from halo, C₁₋₆ alkyl, C₁₋₆ haloalkyl, C₂₋₆ alkenyl, C₂₋₆ alkynyl, and NR^{c11}R^{d11}; and
each R^{c11} and R^{d11} is independently selected from H, C₁₋₆ alkyl, C₁₋₆ haloalkyl, C₂₋₆ alkenyl, and C₂₋₆ alkynyl.

In some embodiments, each R^{1A} is independently selected from halo, C₁₋₆ alkyl, C₁₋₆ haloalkyl, C₂₋₆ alkenyl, C₂₋₆ alkynyl, and NR^{c11}R^{d11}; and

each R^{c11} and R^{d11} is independently selected from H and C₁₋₆ alkyl.

In some embodiments, each R^{1A} is NR^{c11}R^{d11}.

In some embodiments, each R^{1A} is NR^{c11}R^{d11}; and

each R^{c11} and R^{d11} is independently selected from H, C₁₋₆ alkyl, C₁₋₆ haloalkyl, C₂₋₆ alkenyl, and C₂₋₆ alkynyl.

In some embodiments, each R^{1A} is NR^{c11}R^{d11}; and

each R^{c11} and R^{d11} is independently selected from H and C₁₋₆ alkyl.

In some embodiments, each R^{1A} is NH₂.

In some embodiments, R¹ is methyl or aminopropyl.

In some embodiments, R¹ is C₁₋₆ alkyl.

In some embodiments, R¹ is C₁₋₃ alkyl.

In some embodiments, R¹ is methyl.

In some embodiments, R² is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{2A} substituents.

In some embodiments, R² is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R² are each optionally substituted with 1 or 2 independently selected R^{2A} substituents.

In some embodiments, R² is C₁₋₆ alkyl, which is optionally substituted with 1, 2, 3, or 4 independently selected R^{2A} substituents.

In some embodiments, R² is C₁₋₆ alkyl, which is optionally substituted with 1 or 2 independently selected R^{2A} substituents.

In some embodiments, R² is C₁₋₆ alkyl, which is optionally substituted with 1 or 2 independently selected R^{2A} substituents.

In some embodiments, R² is C₁₋₃ alkyl, which is optionally substituted with 1 or 2 independently selected R^{2A} substituents.

In some embodiments, R² is methyl or propyl, each of which is optionally substituted with 1 or 2 independently selected R^{2A} substituents.

In some embodiments, each R^{2A} is independently selected from halo, C₁₋₆ alkyl, C₁₋₆ haloalkyl, C₂₋₆ alkenyl, C₂₋₆ alkynyl, and NR^{c21}R^{d2}.

In some embodiments, each R^{2A} is NR^{c21}R^{d21}.

In some embodiments, each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl.

In some embodiments, each R^{a21}, R^{b21}, R^{c21}, and R^{d21} is independently selected from H and C₁₋₆ alkyl.

In some embodiments, each R^{c21} and R^{d21} is independently selected from H, C₁₋₆ alkyl, C₁₋₆ haloalkyl, C₂₋₆ alkenyl, and C₂₋₆ alkynyl.

In some embodiments, each R^{c21} and R^{d21} is independently selected from H and C₁₋₆ alkyl.

In some embodiments, each R^{c21} and R^{d21} is H.

In some embodiments, each R^{2A} is independently selected from halo, C₁₋₆ alkyl, C₁₋₆ haloalkyl, C₂₋₆ alkenyl, C₂₋₆ alkynyl, and NR^{c21}R^{d21}; and

each R^{c21} and R^{d21} is independently selected from H, C₁₋₆ alkyl, C₁₋₆ haloalkyl, C₂₋₆ alkenyl, and C₂₋₆ alkynyl.

In some embodiments, each R^{2A} is independently selected from halo, C₁₋₆ alkyl, C₁₋₆ haloalkyl, C₂₋₆ alkenyl, C₂₋₆ alkynyl, and NR^{c21}R^{d21}; and

each R^{c21} and R^{d21} is independently selected from H and C₁₋₆ alkyl.

In some embodiments, each R^{2A} is NR^{c21}R^{d21}; and

each R^{c21} and R^{d21} is independently selected from H, C₁₋₆ alkyl, C₁₋₆ haloalkyl, C₂₋₆ alkenyl, and C₂₋₆ alkynyl.

In some embodiments, each R^{2A} is NR^{c21}R^{d21}; and

each R^{c21} and R^{d21} is independently selected from H and C₁₋₆ alkyl.

In some embodiments, each R^{2A} is NH₂.

In some embodiments, R² is methyl or aminopropyl.

In some embodiments, R² is aminopropyl.

In some embodiments:
R¹ is C₁₋₆ alkyl; and
R² is C₁₋₆ alkyl, which is optionally substituted with 1, 2, 3, or 4 independently selected R^{2A} substituents.

In some embodiments:
R¹ is C₁₋₃ alkyl; and
R² is C₁₋₆ alkyl, which is optionally substituted with 1 or 2 independently selected R^{2A} substituents.

In some embodiments:
R¹ is C₁₋₃ alkyl;
R² is C₁₋₆ alkyl, which is optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, and NR^{c21}R^{d21}; and
each R^{c21} and R^{d21} is independently selected from H, C₁₋₆ alkyl, C₁₋₆ haloalkyl, C₂₋₆ alkenyl, and C₂₋₆ alkynyl.

In some embodiments:
R¹ is C₁₋₃ alkyl;
R² is C₁₋₆ alkyl, which is optionally substituted with 1 or 2 independently selected R^{2A} substituents;
each R^{2A} is NR^{c21}R^{d21}; and
each R^{c21} and R^{d21} is independently selected from H and C₁₋₆ alkyl.

In some embodiments, R¹ is methyl and R² is aminopropyl.

In some embodiments, R¹ and R², together with the N atom to which they are attached, form a 4-12 membered heterocycloalkyl, wherein the 4-12 membered heterocycloalkyl is optionally substituted with 1, 2, 3, 4, 5, 6, 7, or 8 independently selected R¹³ substituents.

In some embodiments, R¹ and R², together with the N atom to which they are attached, form a 4-12 membered heterocycloalkyl, wherein the 4-12 membered heterocycloalkyl is optionally substituted with 1, 2, 3, or 4 independently selected R¹³ substituents.

In some embodiments, R¹ and R², together with the N atom to which they are attached, form a 4-7 membered monocyclic heterocycloalkyl or an 8-12 membered spirocyclic heterocycloalkyl, wherein the 4-7 membered monocyclic heterocycloalkyl and 8-12 membered spirocyclic heterocycloalkyl are each optionally substituted with 1, 2, 3, or 4 independently selected R¹³ substituents.

In some embodiments, R¹ and R², together with the N atom to which they are attached, form a 4-7 membered monocyclic heterocycloalkyl or an 8-12 membered spirocyclic heterocycloalkyl, wherein the 4-7 membered monocyclic heterocycloalkyl and 8-12 membered spirocyclic heterocycloalkyl are each optionally substituted with 1 or 2 independently selected R¹³ substituents.

In some embodiments, R¹ and R², together with the N atom to which they are attached, form a 4-7 membered monocyclic heterocycloalkyl, wherein the 4-7 membered monocyclic heterocycloalkyl is optionally substituted with 1, 2, 3, or 4 independently selected R¹³ substituents.

In some embodiments, R¹ and R², together with the N atom to which they are attached, form a 4-7 membered monocyclic heterocycloalkyl, wherein the 4-7 membered monocyclic heterocycloalkyl is optionally substituted with 1 or 2 independently selected R¹³ substituents.

In some embodiments, R¹ and R², together with the N atom to which they are attached, form a pyrrolidinyl or piperidinyl, wherein the pyrrolidinyl and piperidinyl are each optionally substituted with 1, 2, 3, or 4 independently selected R¹³ substituents.

In some embodiments, R¹ and R², together with the N atom to which they are attached, form a pyrrolidinyl or piperidinyl, wherein the pyrrolidinyl and piperidinyl are each optionally substituted with 1 or 2 independently selected R¹³ substituents.

In some embodiments, R¹ and R², together with the N atom to which they are attached, form an 8-12 membered spirocyclic heterocycloalkyl, wherein the 8-12 membered spirocyclic heterocycloalkyl is optionally substituted with 1, 2, 3, or 4 independently selected R¹³ substituents.

In some embodiments, R¹ and R², together with the N atom to which they are attached, form an 8-12 membered spirocyclic heterocycloalkyl, wherein the 8-12 membered spirocyclic heterocycloalkyl is optionally substituted with 1 or 2 independently selected R¹³ substituents.

In some embodiments, R¹ and R², together with the N atom to which they are attached, form 2,9-diazaspiro[5.5]undecanyl or 1-oxa-4,9-diazaspiro[5.5]undecanyl, wherein the 2,9-diazaspiro[5.5]undecanyl and 1-oxa-4,9-diazaspiro[5.5]undecanyl are each optionally substituted with 1, 2, 3, or 4 independently selected R¹³ substituents.

In some embodiments, R¹ and R², together with the N atom to which they are attached, form 2,9-diazaspiro[5.5]undecanyl, wherein the 2,9-diazaspiro[5.5]undecanyl is optionally substituted with 1, 2, 3, or 4 independently selected R¹³ substituents.

In some embodiments, R¹ and R², together with the N atom to which they are attached, form 2,9-diazaspiro[5.5]undecanyl, wherein the 2,9-diazaspiro[5.5]undecanyl is optionally substituted with 1 or 2 independently selected R¹³ substituents.

In some embodiments, R¹ and R², together with the N atom to which they are attached, form a 1-oxa-4,9-diazaspiro[5.5]undecanyl, wherein the 1-oxa-4,9-diazaspiro[5.5]undecanyl is optionally substituted with 1, 2, 3, or 4 independently selected R¹³ substituents.

In some embodiments, R¹ and R², together with the N atom to which they are attached, form a 1-oxa-4,9-diazaspiro[5.5]undecanyl, wherein the 1-oxa-4,9-diazaspiro[5.5]undecanyl is optionally substituted with 1 or 2 independently selected R¹³ substituents.

In some embodiments, R¹ and R², together with the N atom to which they are attached, form a pyrrolidinyl, piperidinyl, 2,9-diazaspiro[5.5]undecanyl, or 1-oxa-4,9-diazaspiro[5.5]undecanyl, wherein the pyrrolidinyl, piperidinyl, 2,9-diazaspiro[5.5]undecanyl, and 1-oxa-4,9-diazaspiro[5.5]undecanyl are each optionally substituted with 1, 2, 3, or 4 independently selected R¹³ substituents.

In some embodiments, R¹ and R², together with the N atom to which they are attached, form a pyrrolidinyl, piperidinyl, or 1-oxa-4,9-diazaspiro[5.5]undecanyl, wherein the pyrrolidinyl, piperidinyl, and 1-oxa-4,9-diazaspiro[5.5]undecanyl are each optionally substituted with 1, 2, 3, or 4 independently selected R¹³ substituents.

In some embodiments, R¹ and R², together with the N atom to which they are attached, form a pyrrolidinyl, piperidinyl, 2,9-diazaspiro[5.5]undecanyl, or 1-oxa-4,9-diazaspiro[5.5]undecanyl, wherein the pyrrolidinyl, piperidinyl, 2,9-diazaspiro[5.5]undecanyl, and 1-oxa-4,9-diazaspiro[5.5]undecanyl are each optionally substituted with 1 or 2 independently selected R¹³ substituents.

In some embodiments, R¹ and R², together with the N atom to which they are attached, form a pyrrolidinyl, piperidinyl, or 1-oxa-4,9-diazaspiro[5.5]undecanyl, wherein the pyrrolidinyl, piperidinyl, and 1-oxa-4,9-diazaspiro[5.5]undecanyl are each optionally substituted with 1 or 2 independently selected R¹³ substituents.

In some embodiments, each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1, 2, 3, or 4 independently selected R^{13A} substituents.

In some embodiments, each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1 or 2 independently selected R^{13A} substituents.

In some embodiments, each R¹³ is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1 or 2 independently selected R^{13A} substituents.

In some embodiments, each R¹³ is independently selected from halo, oxo, C₁₋₆ alkyl, C₁₋₆ haloalkyl, C₂₋₆ alkenyl, C₂₋₆ alkynyl, C₃₋₁₀ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, C₃₋₁₀ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1, 2, 3, or 4 independently selected R^{13A} substituents.

In some embodiments, each R¹³ is independently selected from halo, oxo, C₁₋₆ alkyl, C₁₋₆ haloalkyl, C₂₋₆ alkenyl, C₂₋₆ alkynyl, C₃₋₁₀ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, C₃₋₁₀ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1 or 2 independently selected R^{13A} substituents.

In some embodiments, each R¹³ is independently selected from halo, oxo, C₁₋₆ alkyl, C₁₋₆ haloalkyl, C₂₋₆ alkenyl, C₂₋₆ alkynyl, C₃₋₇ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, C₃₋₇ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1, 2, 3, or 4 independently selected R^{13A} substituents.

In some embodiments, each R¹³ is independently selected from halo, oxo, C₁₋₆ alkyl, C₁₋₆ haloalkyl, C₂₋₆ alkenyl, C₂₋₆ alkynyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1, 2, 3, or 4 independently selected R^{13A} substituents.

In some embodiments, each R¹³ is independently selected from halo, oxo, C₁₋₆ alkyl, C₁₋₆ haloalkyl, C₂₋₆ alkenyl, C₂₋₆ alkynyl, C₃₋₇ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, C₃₋₇ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1 or 2 independently selected R^{13A} substituents.

In some embodiments, each R¹³ is independently selected from halo, oxo, C₁₋₆ alkyl, C₁₋₆ haloalkyl, C₂₋₆ alkenyl, C₂₋₆ alkynyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1 or 2 independently selected R^{13A} substituents.

In some embodiments, each R¹³ is independently selected from C₁₋₆ alkyl, C₁₋₆ haloalkyl, C₃₋₇ cycloalkyl, phenyl-C₁₋₆ alkyl-, and C₃₋₇ cycloalkyl-C₁₋₆ alkyl-, wherein the C₁₋₆ alkyl, C₃₋₇ cycloalkyl, phenyl-C₁₋₆ alkyl-, and C₃₋₇ cycloalkyl-C₁₋₆ alkyl- of R¹³ are each optionally substituted with 1 or 2 independently selected R^{13A} substituents.

In some embodiments, each R¹³ is independently selected from C₁₋₆ alkyl, C₁₋₆ haloalkyl, phenyl-C₁₋₆ alkyl-, and C₃₋₇ cycloalkyl-C₁₋₆ alkyl-, wherein the C₁₋₆ alkyl, phenyl-C₁₋₆ alkyl-, and C₃₋₇ cycloalkyl-C₁₋₆ alkyl- of R¹³ are each optionally substituted with 1 or 2 independently selected R^{13A} substituents.

In some embodiments, each R¹³ is independently selected from C₁₋₆ alkyl, C₃₋₁₀ cycloalkyl, and C₃₋₁₀ cycloalkyl-C₁₋₆ alkyl-, wherein the C₁₋₆ alkyl, C₃₋₁₀ cycloalkyl, and C₃₋₁₀ cycloalkyl-C₁₋₆ alkyl- of R¹³ are each optionally substituted with 1, 2, 3, or 4 independently selected R^{13A} substituents.

In some embodiments, each R¹³ is independently selected from C₁₋₆ alkyl and C₃₋₁₀ cycloalkyl-C₁₋₆ alkyl-, wherein the C₁₋₆ alkyl and C₃₋₁₀ cycloalkyl-C₁₋₆ alkyl- of R¹³ are each optionally substituted with 1, 2, 3, or 4 independently selected R^{13A} substituents.

In some embodiments, each R¹³ is independently selected from C₁₋₆ alkyl, C₃₋₁₀ cycloalkyl, and C₃₋₁₀ cycloalkyl-C₁₋₆ alkyl-, wherein the C₁₋₆ alkyl, C₃₋₁₀ cycloalkyl, and C₃₋₁₀ cycloalkyl-C₁₋₆ alkyl- of R¹³ are each optionally substituted with 1 or 2 independently selected R^{13A} substituents.

In some embodiments, each R¹³ is independently selected from C₁₋₆ alkyl and C₃₋₁₀ cycloalkyl-C₁₋₆ alkyl-, wherein the C₁₋₆ alkyl and C₃₋₁₀ cycloalkyl-C₁₋₆ alkyl- of R¹³ are each optionally substituted with 1 or 2 independently selected R^{13A} substituents.

In some embodiments, each R¹³ is independently selected from C₁₋₆ alkyl, C₃₋₇ cycloalkyl, and C₃₋₇ cycloalkyl-C₁₋₆ alkyl-, wherein the C₁₋₆ alkyl, C₃₋₇ cycloalkyl, and C₃₋₇ cycloalkyl-C₁₋₆ alkyl- of R¹³ are each optionally substituted with 1, 2, 3, or 4 independently selected R^{13A} substituents.

In some embodiments, each R¹³ is independently selected from C₁₋₆ alkyl and C₃₋₇ cycloalkyl-C₁₋₆ alkyl-, wherein the C₁₋₆ alkyl and C₃₋₇ cycloalkyl-C₁₋₆ alkyl- of R¹³ are each optionally substituted with 1, 2, 3, or 4 independently selected R^{13A} substituents.

In some embodiments, each R¹³ is independently selected from C₁₋₆ alkyl, C₃₋₇ cycloalkyl, and C₃₋₇ cycloalkyl-C₁₋₆ alkyl-, wherein the C₁₋₆ alkyl, C₃₋₇ cycloalkyl, and C₃₋₇ cycloalkyl-C₁₋₆ alkyl- of R¹³ are each optionally substituted with 1 or 2 independently selected R^{13A} substituents.

In some embodiments, each R¹³ is independently selected from C₁₋₆ alkyl and C₃₋₇ cycloalkyl-C₁₋₆ alkyl-, wherein the C₁₋₆ alkyl and C₃₋₇ cycloalkyl-C₁₋₆ alkyl- of R¹³ are each optionally substituted with 1 or 2 independently selected R^{13A} substituents.

In some embodiments, each R^{13A} is independently selected from halo, C₁₋₆ alkyl, C₁₋₆ haloalkyl, C₂₋₆ alkenyl, C₂₋₆ alkynyl, OR^{a132}, and NR^{c132}R^{a132}.

In some embodiments, each R^{13A} is independently selected from OR^{a132} and NR^{c132}R^{d132}.

In some embodiments, each R^{13A} is OR^{a132}.

In some embodiments, each R^{13A} is NR^{c132}R^{d132}.

In some embodiments, each R^{a132}, R^{b132}, R^{c132}, and R^{d132} is independently selected from H, C₁₋₆ alkyl, C₁₋₆ haloalkyl, C₂₋₆ alkenyl, and C₂₋₆ alkynyl.

In some embodiments, each R^{a132}, R^{b132}, R^{c132} and R^{d132} is independently selected from H and C₁₋₆ alkyl.

In some embodiments, each R^{a132}, R^{b132}, R^{c132} and R^{d132} is independently selected from H and C₁₋₃ alkyl.

In some embodiments, each R^{a132}, R^{c132}, and R^{d132} is independently selected from H, C₁₋₆ alkyl, C₁₋₆ haloalkyl, C₂₋₆ alkenyl, and C₂₋₆ alkynyl.

In some embodiments, each R^{a132}, R^{c132}, and R^{d132} is independently selected from H and C₁₋₆ alkyl.

In some embodiments, each R^{a132}, R^{c132}, and R^{d132} is independently selected from H and C₁₋₃ alkyl.

In some embodiments, each R^{13A} is independently selected from halo, C₁₋₆ alkyl, C₁₋₆ haloalkyl, C₂₋₆ alkenyl, C₂₋₆ alkynyl, OR^{a132}, and NR^{c132}R^{d132}, each R^{a132}, R^{c132}, and R^{d132} is independently selected from H, C₁₋₆ alkyl, C₁₋₆ haloalkyl, C₂₋₆ alkenyl, and C₂₋₆ alkynyl.

In some embodiments, each R^{13A} is independently selected from halo, C₁₋₆ alkyl, C₁₋₆ haloalkyl, C₂₋₆ alkenyl, C₂₋₆ alkynyl, OR^{a132}, and NR^{c132}R^{d132}. each R^{a132}, R^{c132}, and R^{d132} is independently selected from H and C₁₋₆ alkyl.

In some embodiments, each R^{13A} is independently selected amino, hydroxy, and methoxy.

In some embodiments, each R^{13A} is independently selected amino and methoxy.

In some embodiments, each R^{13A} is amino.

In some embodiments, each R^{13A} is hydroxy.

In some embodiments, each R^{13A} is methoxy.

In some embodiments, each R¹³ is independently selected from ethyl, cyclobutyl, cyclopropylmethyl, aminomethyl, hydroxyethyl, and methoxyethyl.

In some embodiments, each R¹³ is independently selected from ethyl, cyclopropylmethyl, aminomethyl, and methoxyethyl.

In some embodiments, R³ is selected from halo and C₁₋₆ haloalkyl.

In some embodiments, R³ is selected from halo and C₁₋₃ haloalkyl.

In some embodiments, R³ is selected from chloro and trifluoromethyl.

In some embodiments, R³ is chloro.

In some embodiments, R³ is trifluoromethyl.

In some embodiments, R⁴ is selected from H, halo, C₁₋₆ alkyl, C₁₋₆ alkoxy, C₁₋₆ haloalkyl, C₁₋₆haloalkoxy, C₂₋₆ alkenyl, and C₂₋₆ alkynyl.

In some embodiments, R⁴ is H.

In some embodiments, L is a bond.

In some embodiments, L is NR¹⁰.

In some embodiments, L is O.

In some embodiments, Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl are each optionally substituted with 1, 2, 3, or 4 independently selected R¹¹ substituents.

In some embodiments, Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl are each optionally substituted with 1 or 2 independently selected R¹¹ substituents.

In some embodiments, Cy¹ is selected from phenyl, 5-6 membered heteroaryl, and C₃₋₇ cycloalkyl, wherein the phenyl, 5-6 membered heteroaryl, and C₃₋₇ cycloalkyl are each optionally substituted with 1, 2, 3, or 4 independently selected R¹¹ substituents.

In some embodiments, Cy¹ is selected from phenyl and C₃₋₇ cycloalkyl, wherein the phenyl and C₃₋₇ cycloalkyl are each optionally substituted with 1, 2, 3, or 4 independently selected R¹¹ substituents.

In some embodiments, Cy¹ is selected from phenyl, 5-6 membered heteroaryl, and C₃₋₇ cycloalkyl, wherein the phenyl, 5-6 membered heteroaryl, and C₃₋₇ cycloalkyl are each optionally substituted with 1 or 2 independently selected R¹¹ substituents.

In some embodiments, Cy¹ is selected from phenyl and C₃₋₇ cycloalkyl, wherein the phenyl and C₃₋₇ cycloalkyl are each optionally substituted with 1 or 2 independently selected R¹¹ substituents.

In some embodiments, Cy¹ is phenyl, wherein the phenyl is optionally substituted with 1, 2, 3, or 4 independently selected R¹¹ substituents.

In some embodiments, Cy¹ is phenyl, wherein the phenyl is optionally substituted with 1 or 2 independently selected R¹¹ substituents.

In some embodiments, Cy¹ is C₃₋₇ cycloalkyl, wherein the C₃₋₇ cycloalkyl is optionally substituted with 1, 2, 3, or 4 independently selected R¹¹ substituents.

In some embodiments, Cy¹ is C₃₋₇ cycloalkyl, wherein the C₃₋₇ cycloalkyl is optionally substituted with 1 or 2 independently selected R¹¹ substituents.

In some embodiments, Cy¹ is 5-6 membered heteroaryl, wherein the 5-6 membered heteroaryl is optionally substituted with 1, 2, 3, or 4 independently selected R¹¹ substituents.

In some embodiments, Cy¹ is 5-6 membered heteroaryl, wherein the 5-6 membered heteroaryl is optionally substituted with 1 or 2 independently selected R¹¹ substituents.

In some embodiments, Cy¹ is selected from phenyl, pentadeuterophenyl, pyridinyl, cyclopropyl, cyclobutyl, cyclopentyl, cyclohexyl, and bicyclo[3.1.1]heptanyl, wherein the phenyl, pyridinyl, cyclopropyl, cyclobutyl, cyclopentyl, cyclohexyl, and bicyclo[3.1.1]heptanyl are each optionally substituted with 1, 2, 3, or 4 independently selected R¹¹ substituents.

In some embodiments, Cy¹ is selected from phenyl and cyclopropyl, wherein the phenyl and cyclopropyl are each optionally substituted with 1, 2, 3, or 4 independently selected R¹¹ substituents.

In some embodiments, Cy¹ is selected from phenyl, pentadeuterophenyl, pyridinyl, cyclopropyl, cyclobutyl, cyclopentyl, cyclohexyl, and bicyclo[3.1.1]heptanyl, wherein the phenyl, pyridinyl, cyclopropyl, cyclobutyl, cyclopentyl, cyclohexyl, and bicyclo[3.1.1]heptanyl are each optionally substituted with 1 or 2 independently selected R¹¹ substituents.

In some embodiments, Cy¹ is selected from phenyl and cyclopropyl, wherein the phenyl and cyclopropyl are each optionally substituted with 1 or 2 independently selected R¹¹ substituents.

In some embodiments, each R¹¹ is independently selected from halo, C₁₋₆ alkyl, C₁₋₆ haloalkyl, C₂₋₆ alkenyl, and OR^{a111}.

In some embodiments, each R¹¹¹ is independently selected from halo and OR^{a111}.

In some embodiments, each R^{a111}, R^{b111}, R^{c111}, and R^{d111} is independently selected from H, C₁₋₆ alkyl, C₁₋₆ haloalkyl, C₂₋₆ alkenyl, and C₂₋₆ alkynyl.

In some embodiments, each R^{a111}, R^{b111}, R^{c111}, and R^{d111} is independently selected from H and C₁₋₆ alkyl.

In some embodiments, each R^{a111} is independently selected from H, C₁₋₆ alkyl, C₁₋₆ haloalkyl, C₂₋₆ alkenyl, and C₂₋₆ alkynyl.

In some embodiments, each R^{a111} is independently selected from H and C₁₋₆ alkyl.

In some embodiments, each R^{a111} is an independently selected C₁₋₆ alkyl.

In some embodiments, each R^{a111} is an independently selected C₁₋₃ alkyl.

In some embodiments, each R¹¹ is independently selected from halo, C₁₋₆ alkyl, C₁₋₆ haloalkyl, C₂₋₆ alkenyl, and OR^{a111}; and

each R^{a111} is independently selected from H and C₁₋₆ alkyl.

In some embodiments, each R¹¹ is independently selected from halo, C₁₋₆ alkyl, C₁₋₆ haloalkyl, C₂₋₆ alkenyl, and OR^{a111}; and

each R^{a111} is an independently selected C₁₋₆ alkyl.

In some embodiments, each R¹¹ is independently selected from halo and OR^{a111}; and

each R^{a111} is independently selected from H and C₁₋₆ alkyl.

In some embodiments, each R¹¹ is independently selected from halo and OR^{a111}; and each R^{a111} is an independently selected C₁₋₆ alkyl.

In some embodiments, each R¹¹ is independently selected from halo and OR^{a111}; and each R^{a111} is an independently selected C₁₋₃ alkyl.

In some embodiments, each R¹¹ is independently selected from fluoro and methoxy.

In some embodiments, each R¹¹ is independently selected from fluoro and methoxy.

In some embodiments, Cy¹ is selected from cyclopropyl, cyclobutyl, cyclopentyl, cyclohexyl, bicyclo[3.1.1]heptanyl, fluoropyridinyl, phenyl, pentadeuterophenyl, fluorophenyl, difluorophenyl, and (fluoro)(methoxy)phenyl.

In some embodiments, Cy¹ is selected from cyclopropyl, phenyl, fluorophenyl, and (fluoro)(methoxy)phenyl.

In some embodiments:
Ring A is:

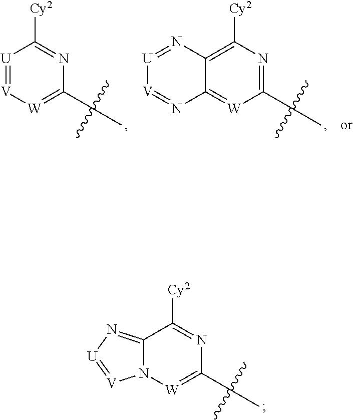

U is CR⁵ or N;
V is CR⁶ or N;
W is CR⁷ or N;
wherein at least one of U, V, or W is not N;
L is a bond, O, or NR¹¹.
Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl are each optionally substituted with 1, 2, 3, or 4 independently selected R¹¹ substituents;
each R¹¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a111}, SR^{a111}, NHOR^{a111}, C(O)R^{b111}, C(O)NR^{c111}R^{d111}, C(O)NR^{c111}(OR^{a111}), C(O)OR^{a111}, OC(O)R^{b111}, OC(O)NR^{c111}R^{d111}, NR^{c111}R^{d111}, NR^{c111}NR^{c111}R^{d111}, NR^{c111}C(O)R^{b111}, NR^{c111}C(O)OR^{a111}, NR^{c111}C(O)NR^{c111}R^{d111}, C(═NR^{e111})R^{b111}, C(═NR^{e111})NR^{c111}R^{d111}, NR^{c111}C(═NR^{e111})NR^{c111}R^{d111}, NR^{c111}C(═NR^{e111})R^{b111}, NR^{c111}S(O)R^{b111}, NR^{c111}S(O)NR^{c111}R^{d111}, NR^{c111} S(O)₂R^{b111}, NR^{c111}S(O)(═NR^{e111})R^{b111}, NR^{c111}S(O)₂NR^{c111}R^{d111}, S(O)R^{b111}, S(O)NR^{c111}R^{d111}, S(O)₂R^{b111}, S(O)₂NR^{c111}R^{d111}, OS(O)(═NR^{e111})R^{b111}, and OS(O)₂R^{b111};
each R^{a111}, R^{c111}, and R^{d111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c111} and R^{d111} attached to the same N atom, together with the N atom to which they are attached, form a 5-10 membered heteroaryl or a 4-10 membered heterocycloalkyl group;
each R^{b111} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e111} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
Cy² is 4-pyridazinyl, 5 membered heteroaryl, or 4-8 membered heterocycloalkyl, wherein the 4-pyridazinyl, 5 membered heteroaryl, and 4-8 membered heterocycloalkyl are each optionally substituted with 1, 2, 3, or 4 independently selected R¹² substituents;
R¹² is selected from halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OR^{a121}, CN, C(O)OH, C(O)NHR^{a121}, and NR^{a121}R^{a121}, wherein the C₁₋₆ alkyl is optionally substituted with OH, CN, and NH₂;
each R^{a121} is independently selected from H, C₁₋₆ alkyl, C₁₋₆ alkoxy, C₁₋₆ haloalkyl, C₁₋₆haloalkoxy, OH, NH₂, and NHC₁₋₆ alkyl, wherein the C₁₋₆ alkyl is optionally substituted with OH, CN, and NH₂;
R¹ is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R¹ are each optionally substituted with 1, 2, 3, or 4 independently selected R^{1A} substituents;
each R^{1A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a111}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11};
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c11} and R^{d11} attached to the same N atom, together with the N atom to which they are attached, form a 5-10 membered heteroaryl or a 4-10 membered heterocycloalkyl group;
each R^{b11} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e11} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
R² is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21};
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c21} and R^{d21} attached to the same N atom, together with the N atom to which they are attached, form a 5-10 membered heteroaryl or a 4-10 membered heterocycloalkyl group;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e21} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or R¹ and R², together with the N atom to which they are attached, form a 4-12 membered heterocycloalkyl, wherein the 4-12 membered heterocycloalkyl is optionally substituted with 1, 2, 3, or 4 independently selected R¹³ substituents;
each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1, 2, 3, or 4 independently selected R^{13A} substituents;
each R^{13A} is independently selected from halo, C₁₋₆ alkyl, C₁₋₆ haloalkyl, C₂₋₆ alkenyl, C₂₋₆ alkynyl, OR^{a132}, and NR^{c132}R^{d132};
each R^{a132}, R^{c132}, and R^{d132} is independently selected from H and C₁₋₆ alkyl;
R³ is selected from H, halo, C₁₋₆ alkyl and C₁₋₆ haloalkyl;
R⁴ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₄ cycloalkyl, OH, CN, C(O)OH, C(O)NH₂, and NH₂; wherein the C₁₋₆ alkyl is optionally substituted with OH, CN, and NH₂;
R⁵ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a51}, and NHR^{a52}, wherein the C₁₋₆ alkyl is optionally substituted with OH, CN, and NH₂;
each R^{a51} is independently selected from H, C₁₋₆ alkyl, C₁₋₆ alkoxy, C₁₋₆ haloalkyl, C₁₋₆haloalkoxy, OH, NH₂, and NHC₁₋₆ alkyl, wherein the C₁₋₆ alkyl is optionally substituted with OH, CN, and NH₂;
each R^{a52} is independently selected from C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R⁶ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, and C(O)NH₂; wherein the C₁₋₆ alkyl is optionally substituted with OH, CN, and NH₂; and
R⁷ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a71}, and NHR^{a72}, wherein the C₁₋₆ alkyl is optionally substituted with OH, CN, and NH₂;
R¹⁰ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NH₂, and NH₂; wherein the C₁₋₆ alkyl, C₂₋₆ alkenyl, and C₂₋₆ alkynyl of R¹⁰ are each optionally substituted with 1, 2, 3, or 4 independently selected R^{M} substituents; and
each R^{M} is independently selected from H, OH, halo, oxo, CN, C(O)OH, NH₂, NO₂, SF₅, C₁₋₆ alkyl, C₁₋₆ alkoxy, C₁₋₆haloalkoxy, C₁₋₆haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl.

In some embodiments:
Ring A is:

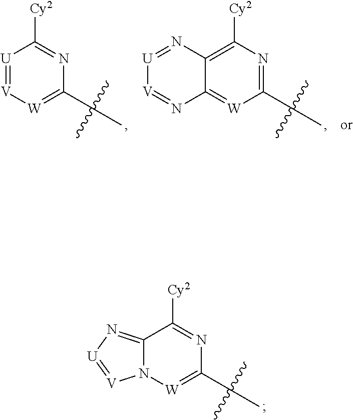

U is CR⁵ or N;
V is CR⁶ or N;
W is CR⁷ or N;
wherein at least one of U, V, or W is not N;
L is a bond, O, or NR¹⁰;
Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl are each optionally substituted with 1, 2, 3, or 4 independently selected R¹¹ substituents;
R¹¹ is independently selected from halo, C₁₋₆ alkyl, C₁₋₆ haloalkyl, C₂₋₆ alkenyl, and OR^{a111};
each R^{a111} is independently selected from H and C₁₋₆ alkyl;
Cy² is 4-pyridazinyl, 5 membered heteroaryl, or 4-6 membered heterocycloalkyl, wherein the 4-pyridazinyl, 5 membered heteroaryl, and 4-6 membered heterocycloalkyl are each optionally substituted with 1, 2, 3, or 4 independently selected R¹² substituents;
R¹² is independently selected from halo, C₁₋₆ alkyl, C₁₋₆ alkoxy, C₁₋₆ haloalkyl, C₁₋₆ haloalkoxy, C₂₋₆ alkenyl, and C₂₋₆ alkynyl;
R¹ is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R¹ are each optionally substituted with 1, 2, 3, or 4 independently selected R^{1A} substituents;
each R^{1A} is independently selected from halo, C₁₋₆ alkyl, C₁₋₆ haloalkyl, C₂₋₆ alkenyl, C₂₋₆ alkynyl, and NR^{c11}R^{d11};
each R^{c11} and R^{d11} is independently selected from H and C₁₋₆ alkyl;
R² is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, and NR^{c21}R^{d21};
each R^{c21} and R^{d21} is independently selected from H and C₁₋₆ alkyl;
or R¹ and R², together with the N atom to which they are attached, form a 4-12 membered heterocycloalkyl, wherein the 4-12 membered heterocycloalkyl is optionally substituted with 1, 2, 3, or 4 independently selected R¹³ substituents;
each R¹³ is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1, 2, 3, or 4 independently selected R^{13A} substituents; each R^{13A} is independently selected from halo, C₁₋₆ alkyl, C₁₋₆ haloalkyl, C₂₋₆ alkenyl, C₂₋₆ alkynyl, OR^{a132}, and NR^{c132}R^{d132};
each R^{a132}, R^{c132}, and R^{d132} is independently selected from H and C₁₋₆ alkyl;
R³ is selected from halo and C₁₋₆ haloalkyl;
R⁴ is selected from H, halo, C₁₋₆ alkyl, C₁₋₆ alkoxy, C₁₋₆ haloalkyl, C₁₋₆ haloalkoxy, C₂₋₆ alkenyl, and C₂₋₆ alkynyl;
R⁵ is selected from H and C₁₋₆ alkyl;
R⁶ is selected from H and C₁₋₆ alkyl;
R⁷ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, and CN;
R¹⁰ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NH₂, and NH₂; wherein the C₁₋₆ alkyl, C₂₋₆ alkenyl, and C₂₋₆ alkynyl of R¹⁰ are each optionally substituted with 1, 2, 3, or 4 independently selected R^{M} substituents; and
each R^{M} is independently selected from H, OH, halo, oxo, CN, C(O)OH, NH₂, NO₂, SF₅, C₁₋₆ alkyl, C₁₋₆ alkoxy, C₁₋₆haloalkoxy, C₁₋₆haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl.

In some embodiments, the compound of Formula I is a compound of Formula II:

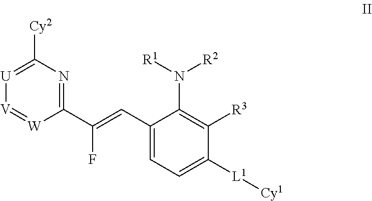

or a pharmaceutically acceptable salt thereof.

In some embodiments, the compound of Formula I is a compound of Formula IIa:

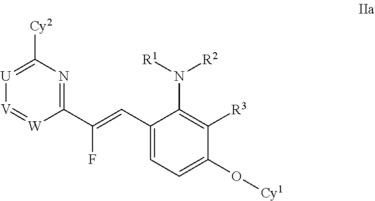

or a pharmaceutically acceptable salt thereof.

In some embodiments, the compound of Formula I is a compound of Formula IIb:

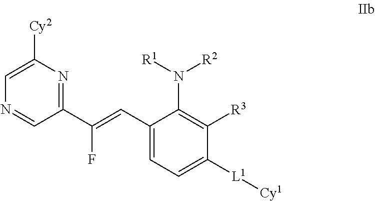

or a pharmaceutically acceptable salt thereof.

In some embodiments, the compound of Formula I is a compound of Formula IIc:

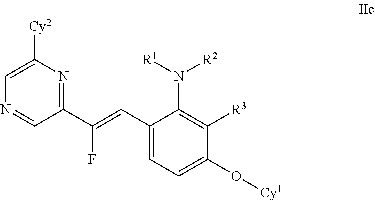

or a pharmaceutically acceptable salt thereof.

In some embodiments, the compound of Formula I is a compound of Formula IId:

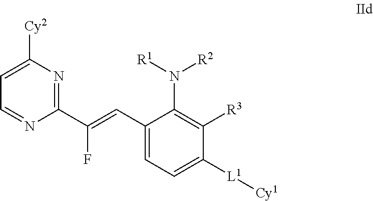

or a pharmaceutically acceptable salt thereof.

In some embodiments, the compound of Formula I is a compound of Formula Ile:

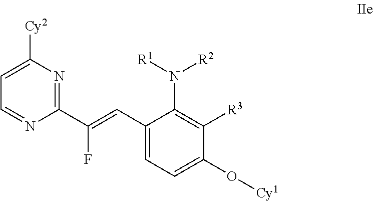

or a pharmaceutically acceptable salt thereof.

In some embodiments, the compound of Formula I is a compound of Formula IIf:

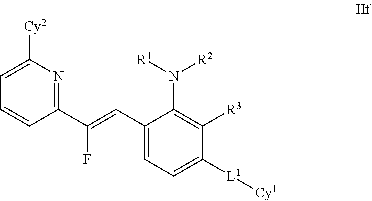

or a pharmaceutically acceptable salt thereof.

In some embodiments, the compound of Formula I is a compound of Formula IIg:

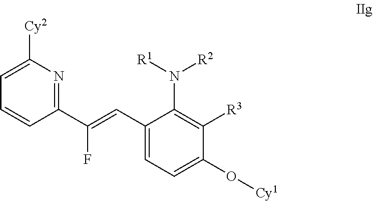

or a pharmaceutically acceptable salt thereof.

In some embodiments, the compound of Formula I is a compound of Formula IIh:

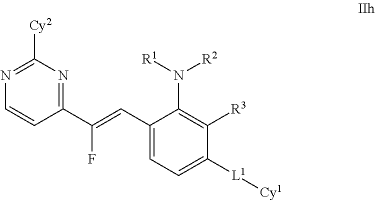

or a pharmaceutically acceptable salt thereof.

In some embodiments, the compound of Formula I is a compound of Formula IIi:

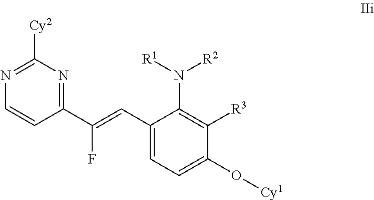

or a pharmaceutically acceptable salt thereof.

In some embodiments, the compound of Formula I is a compound of Formula IIj:

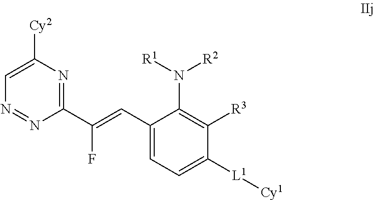

or a pharmaceutically acceptable salt thereof.

In some embodiments, the compound of Formula I is a compound of Formula IIk:

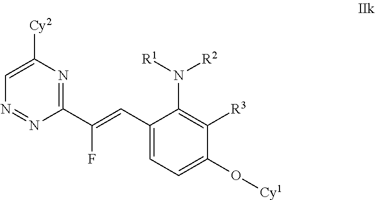

or a pharmaceutically acceptable salt thereof.

In some embodiments, the compound of Formula I is a compound of Formula III:

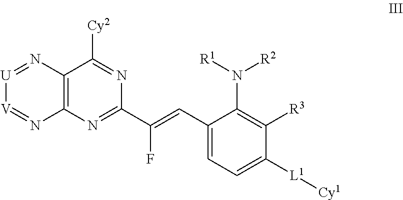

or a pharmaceutically acceptable salt thereof.

In some embodiments, the compound of Formula I is a compound of Formula IIIa:

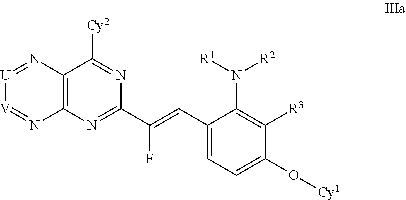

or a pharmaceutically acceptable salt thereof.

In some embodiments, the compound of Formula I is a compound of Formula

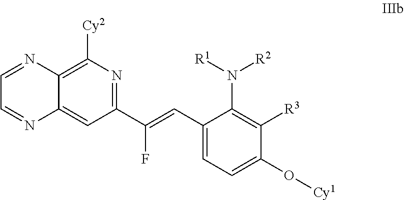

or a pharmaceutically acceptable salt thereof.

In some embodiments, the compound of Formula I is a compound of Formula IV:

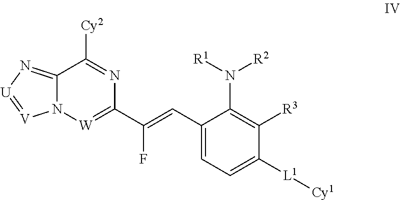

or a pharmaceutically acceptable salt thereof.

In some embodiments, the compound of Formula I is a compound of Formula IVa:

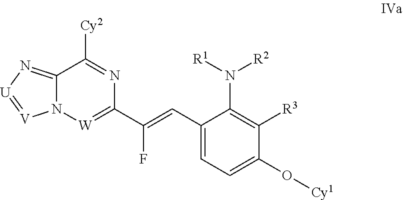

or a pharmaceutically acceptable salt thereof.

In some embodiments, the compound of Formula I is a compound of Formula IVb:

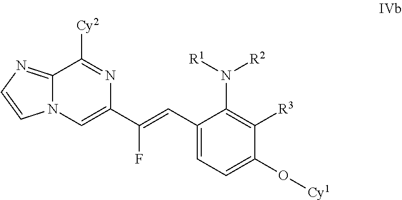

or a pharmaceutically acceptable salt thereof.

In some embodiments, the compound provided herein is selected from:
(S,Z)-(1-(6-(2-(5-(3,3-difluoroazetidin-1-yl)pyrido[3,4-b]pyrazin-7-yl)-2-fluorovinyl)-3-(2-fluorophenoxy)-2-(trifluoromethyl)phenyl)piperidin-3-yl)methanamine;
(S,Z)-(1-(6-(2-(8-(3,3-difluoroazetidin-1-yl)imidazo[1,2-a]pyrazin-6-yl)-2-fluorovinyl)-3-(2-fluorophenoxy)-2-(trifluoromethyl)phenyl)piperidin-3-yl)methanamine;
(S,Z)-3-(2-(2-(3-(aminomethyl)piperidin-1-yl)-4-(2-fluorophenoxy)-3-(trifluoromethyl)phenyl)-1-fluorovinyl)-5-(3,3-difluoroazetidin-1-yl)pyrazine-2-carbonitrile;
(S,Z)-(1-(6-(2-fluoro-2-(6-(isothiazol-5-yl)pyrazin-2-yl)vinyl)-3-(2-fluorophenoxy)-2-(trifluoromethyl)phenyl)piperidin-3-yl)methanamine;
(Z)-N1-(6-(2-fluoro-2-(6-(pyridazin-4-yl)pyridin-2-yl)vinyl)-3-phenoxy-2-(trifluoromethyl)phenyl)-N1-methylpropane-1,3-diamine;
(S,Z)-(1-(2-chloro-6-(2-fluoro-2-(4-(pyridazin-4-yl)pyrimidin-2-yl)vinyl)-3-(4-fluoro-2-methoxyphenoxy)phenyl)piperidin-3-yl)methanamine;
(S,Z)-(1-(2-chloro-6-(2-fluoro-2-(2-(pyridazin-4-yl)pyrimidin-4-yl)vinyl)-3-(4-fluoro-2-methoxyphenoxy)phenyl)piperidin-3-yl)methanamine;
(S,Z)-(1-(2-chloro-6-(2-(5-(3,3-difluoropyrrolidin-1-yl)-1,2,4-triazin-3-yl)-2-fluorovinyl)-3-(4-fluoro-2-methoxyphenoxy)phenyl)piperidin-3-yl)methanamine;
(Z)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane;
(Z)-9-(cyclopropylmethyl)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane;
(Z)-4-(6-(2-fluoro-2-(6-(pyridazin-4-yl)pyrazin-2-yl)vinyl)-3-phenoxy-2-(trifluoromethyl)phenyl)-9-(2-methoxyethyl)-1-oxa-4,9-diazaspiro[5.5]undecane;
(R,Z)-(1-(2-chloro-6-(2-fluoro-2-(6-(pyridazin-4-yl)pyrazin-2-yl)vinyl)-3-phenoxyphenyl)pyrrolidin-2-yl)methanamine;
(R,Z)-(1-(2-chloro-6-(2-fluoro-2-(4-(pyridazin-4-yl)pyrimidin-2-yl)vinyl)-3-phenoxyphenyl)pyrrolidin-2-yl)methanamine;
(Z)-4-(3-cyclopropoxy-6-(2-fluoro-2-(6-(pyridazin-4-yl)pyrazin-2-yl)vinyl)-2-(trifluoromethyl)phenyl)-9-ethyl-1-oxa-4,9-diazaspiro[5.5]undecane;
(Z)-9-(cyclopropylmethyl)-2-(6-(2-(6-(3,3-difluoroazetidin-1-yl)pyrazin-2-yl)-2-fluorovinyl)-3-phenoxy-2-(trifluoromethyl)phenyl)-2,9-diazaspiro[5.5]undecane;
(Z)-2-(6-(2-(6-(3,3-difluoroazetidin-1-yl)pyrazin-2-yl)-2-fluorovinyl)-3-phenoxy-2-(trifluoromethyl)phenyl)-9-ethyl-2,9-diazaspiro[5.5]undecane;
(Z)-9-(cyclopropylmethyl)-2-(6-(2-(6-(3,3-difluoroazetidin-1-yl)pyrazin-2-yl)-2-fluorovinyl)-3-(phenoxy-d₅)-2-(trifluoromethyl)phenyl)-2,9-diazaspiro[5.5]undecane;
(Z)-2-(6-(2-(6-(3,3-difluoroazetidin-1-yl)pyrazin-2-yl)-2-fluorovinyl)-3-(phenoxy-d₅)-2-(trifluoromethyl)phenyl)-9-ethyl-2,9-diazaspiro[5.5]undecane;
(Z)-9-(cyclopropylmethyl)-2-(6-(2-(6-(3,3-difluoropyrrolidin-1-yl)pyrazin-2-yl)-2-fluorovinyl)-3-phenoxy-2-(trifluoromethyl)phenyl)-2,9-diazaspiro[5.5]undecane;
(Z)-2-(6-(2-(6-(3,3-difluoropyrrolidin-1-yl)pyrazin-2-yl)-2-fluorovinyl)-3-phenoxy-2-(trifluoromethyl)phenyl)-9-ethyl-2,9-diazaspiro[5.5]undecane;
(Z)-9-(cyclopropylmethyl)-2-(6-(2-(6-(3,3-difluoropyrrolidin-1-yl)pyrazin-2-yl)-2-fluorovinyl)-3-(phenoxy-d₅)-2-(trifluoromethyl)phenyl)-2,9-diazaspiro[5.5]undecane;
(Z)-2-(6-(2-(6-(3,3-difluoropyrrolidin-1-yl)pyrazin-2-yl)-2-fluorovinyl)-3-(phenoxy-d₅)-2-(trifluoromethyl)phenyl)-9-ethyl-2,9-diazaspiro[5.5]undecane;
(Z)-9-(cyclopropylmethyl)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane;
(Z)-9-ethyl-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane;
(Z)-4-(6-(2-fluoro-2-(4-(pyridazin-4-yl)pyrimidin-2-yl)vinyl)-3-phenoxy-2-(trifluoromethyl)phenyl)-9-(2-methoxyethyl)-1-oxa-4,9-diazaspiro[5.5]undecane;
(Z)-4-(6-(2-fluoro-2-(6-(pyridazin-4-yl)pyridin-2-yl)vinyl)-3-phenoxy-2-(trifluoromethyl)phenyl)-9-(2-methoxyethyl)-1-oxa-4,9-diazaspiro[5.5]undecane;
(Z)-9-(cyclopropylmethyl)-4-(6-(2-fluoro-2-(6-(pyridazin-4-yl)pyridin-2-yl)vinyl)-3-phenoxy-2-(trifluoromethyl)phenyl)-1-oxa-4,9-diazaspiro[5.5]undecane;
(Z)-9-(cyclopropylmethyl)-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane;
(Z)-9-(cyclopropylmethyl)-2-(6-(2-fluoro-2-(6-(pyridazin-4-yl)pyrazin-2-yl)vinyl)-3-phenoxy-2-(trifluoromethyl)phenyl)-2,9-diazaspiro[5.5]undecane;
(Z)-9-cyclobutyl-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane;
(Z)-2-(6-(2-fluoro-2-(4-(pyridazin-4-yl)pyrimidin-2-yl)vinyl)-3-phenoxy-2-(trifluoromethyl)phenyl)-9-(2-methoxyethyl)-2,9-diazaspiro[5.5]undecane;
(Z)-9-ethyl-2-(6-(2-fluoro-2-(4-(pyridazin-4-yl)pyrimidin-2-yl)vinyl)-3-((3-fluoropyridin-2-yl)oxy)-2-(trifluoromethyl)phenyl)-2,9-diazaspiro[5.5]undecane;
(Z)-9-ethyl-2-(6-(2-fluoro-2-(6-(pyridazin-4-yl)pyridin-2-yl)vinyl)-3-((3-fluoropyridin-2-yl)oxy)-2-(trifluoromethyl)phenyl)-2,9-diazaspiro[5.5]undecane;
(Z)-9-ethyl-4-(6-(2-fluoro-2-(6-(pyridazin-4-yl)pyrazin-2-yl)vinyl)-3-(3-fluorophenoxy)-2-(trifluoromethyl)phenyl)-1-oxa-4,9-diazaspiro[5.5]undecane;
(Z)-9-ethyl-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane;
(Z)-9-(cyclopropylmethyl)-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane;
(Z)-9-(cyclopropylmethyl)-2-(6-(2-fluoro-2-(6-(pyridazin-4-yl)pyrazin-2-yl)vinyl)-3-(2-fluorophenoxy)-2-(trifluoromethyl)phenyl)-2,9-diazaspiro[5.5]undecane;
(Z)-9-(cyclopropylmethyl)-2-(3-(2,4-difluorophenoxy)-6-(2-fluoro-2-(6-(pyridazin-4-yl)pyrazin-2-yl)vinyl)-2-(trifluoromethyl)phenyl)-2,9-diazaspiro[5.5]undecane;
(Z)-9-(cyclopropylmethyl)-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane;
(Z)-9-ethyl-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane;
(Z)-9-ethyl-4-(6-(2-fluoro-2-(4-(pyridazin-4-yl)pyrimidin-2-yl)vinyl)-3-(2-fluorophenoxy)-2-(trifluoromethyl)phenyl)-1-oxa-4,9-diazaspiro[5.5]undecane;
(Z)-9-(cyclopropylmethyl)-4-(6-(2-fluoro-2-(4-(pyridazin-4-yl)pyrimidin-2-yl)vinyl)-3-(2-fluorophenoxy)-2-(trifluoromethyl)phenyl)-1-oxa-4,9-diazaspiro[5.5]undecane;
(S,Z)-(1-(6-(2-fluoro-2-(4-(pyridazin-4-yl)pyrimidin-2-yl)vinyl)-3-phenoxy-2-(trifluoromethyl)phenyl)piperidin-3-yl)methanamine;
(Z)-2-(6-(2-(6-(3,3-difluoroazetidin-1-yl)pyrazin-2-yl)-2-fluorovinyl)-3-(2-fluorophenoxy)-2-(trifluoromethyl)phenyl)-9-ethyl-2,9-diazaspiro[5.5]undecane;
(Z)-2-(2-(6-(2-fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecan-9-yl)ethan-1-ol;
(Z)-4-(2-(2-(2-(9-ethyl-2,9-diazaspiro[5.5]undecan-2-yl)-4-(2-fluorophenoxy)-3-(trifluoromethyl)phenyl)-1-fluorovinyl)pyrimidin-4-yl)isothiazole;
(Z)-4-(6-(2-(2-(9-(cyclopropylmethyl)-2,9-diazaspiro[5.5]undecan-2-yl)-4-(2,3-difluorophenoxy)-3-(trifluoromethyl)phenyl)-1-fluorovinyl)pyrazin-2-yl)isothiazole;
(Z)-2-(3-(cyclohexyloxy)-6-(2-fluoro-2-(6-(pyridazin-4-yl)pyrazin-2-yl)vinyl)-2-(trifluoromethyl)phenyl)-9-(cyclopropylmethyl)-2,9-diazaspiro[5.5]undecane;
(Z)-5-(6-(2-(2-(9-(cyclopropylmethyl)-2,9-diazaspiro[5.5]undecan-2-yl)-4-(2,3-difluorophenoxy)-3-(trifluoromethyl)phenyl)-1-fluorovinyl)pyrazin-2-yl)isothiazole;
(Z)-2-(3-(cyclohexyloxy)-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane;
(Z)-2-(3-(cyclopentyloxy)-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane;
(Z)-2-(3-cyclobutoxy-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane;
(Z)-2-(3-(cyclopentyloxy)-6-(2-fluoro-2-(4-(pyridazin-4-yl)pyrimidin-2-yl)vinyl)-2-(trifluoromethyl)phenyl)-9-ethyl-2,9-diazaspiro[5.5]undecane;
(Z)-2-(3-(bicyclo[3.1.1]heptan-3-yloxy)-6-(2-fluoro-2-(4-(pyridazin-4-yl)pyrimidin-2-yl)vinyl)-2-(trifluoromethyl)phenyl)-9-ethyl-2,9-diazaspiro[5.5]undecane;
(Z)-4-(2-(2-(2-(9-(cyclopropylmethyl)-2,9-diazaspiro[5.5]undecan-2-yl)-4-(2-fluorophenoxy)-3-(trifluoromethyl)phenyl)-1-fluorovinyl)pyrimidin-4-yl)isothiazole; and
(Z)-4-(6-(2-(2-(9-(cyclopropylmethyl)-2,9-diazaspiro[5.5]undecan-2-yl)-4-(2-fluorophenoxy)-3-(trifluoromethyl)phenyl)-1-fluorovinyl)pyrazin-2-yl)isothiazole; or a pharmaceutically acceptable salt thereof.

In some embodiments, the compound provided herein is selected from:
(Z)-9-(cyclopropylmethyl)-2-(6-(2-(6-(3,3-difluoroazetidin-1-yl)pyrazin-2-yl)-2-fluorovinyl)-3-phenoxy-2-(trifluoromethyl)phenyl)-2,9-diazaspiro[5.5]undecane;
(Z)-2-(6-(2-(6-(3,3-difluoroazetidin-1-yl)pyrazin-2-yl)-2-fluorovinyl)-3-phenoxy-2-(trifluoromethyl)phenyl)-9-ethyl-2,9-diazaspiro[5.5]undecane;
(Z)-2-(6-(2-(6-(3,3-difluoroazetidin-1-yl)pyrazin-2-yl)-2-fluorovinyl)-3-(phenoxy-d₅)-2-(trifluoromethyl)phenyl)-9-ethyl-2,9-diazaspiro[5.5]undecane;
(Z)-2-(6-(2-(6-(3,3-difluoropyrrolidin-1-yl)pyrazin-2-yl)-2-fluorovinyl)-3-phenoxy-2-(trifluoromethyl)phenyl)-9-ethyl-2,9-diazaspiro[5.5]undecane;
(Z)-2-(6-(2-(6-(3,3-difluoropyrrolidin-1-yl)pyrazin-2-yl)-2-fluorovinyl)-3-(phenoxy-d₅)-2-(trifluoromethyl)phenyl)-9-ethyl-2,9-diazaspiro[5.5]undecane;
(Z)-9-(cyclopropylmethyl)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane;
(Z)-9-(cyclopropylmethyl)-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane;
(Z)-9-(cyclopropylmethyl)-2-(6-(2-fluoro-2-(6-(pyridazin-4-yl)pyrazin-2-yl)vinyl)-3-phenoxy-2-(trifluoromethyl)phenyl)-2,9-diazaspiro[5.5]undecane;
(Z)-2-(6-(2-fluoro-2-(4-(pyridazin-4-yl)pyrimidin-2-yl)vinyl)-3-phenoxy-2-(trifluoromethyl)phenyl)-9-(2-methoxyethyl)-2,9-diazaspiro[5.5]undecane;
(Z)-9-ethyl-2-(6-(2-fluoro-2-(4-(pyridazin-4-yl)pyrimidin-2-yl)vinyl)-3-((3-fluoropyridin-2-yl)oxy)-2-(trifluoromethyl)phenyl)-2,9-diazaspiro[5.5]undecane;
(Z)-9-ethyl-4-(6-(2-fluoro-2-(6-(pyridazin-4-yl)pyrazin-2-yl)vinyl)-3-(3-fluorophenoxy)-2-(trifluoromethyl)phenyl)-1-oxa-4,9-diazaspiro[5.5]undecane;
(Z)-9-(cyclopropylmethyl)-2-(3-(2,4-difluorophenoxy)-6-(2-fluoro-2-(6-(pyridazin-4-yl)pyrazin-2-yl)vinyl)-2-(trifluoromethyl)phenyl)-2,9-diazaspiro[5.5]undecane;
(Z)-9-(cyclopropylmethyl)-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane;
(Z)-9-ethyl-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane;
(Z)-9-ethyl-4-(6-(2-fluoro-2-(4-(pyridazin-4-yl)pyrimidin-2-yl)vinyl)-3-(2-fluorophenoxy)-2-(trifluoromethyl)phenyl)-1-oxa-4,9-diazaspiro[5.5]undecane;
(Z)-9-(cyclopropylmethyl)-4-(6-(2-fluoro-2-(4-(pyridazin-4-yl)pyrimidin-2-yl)vinyl)-3-(2-fluorophenoxy)-2-(trifluoromethyl)phenyl)-1-oxa-4,9-diazaspiro[5.5]undecane;
(Z)-2-(6-(2-(6-(3,3-difluoroazetidin-1-yl)pyrazin-2-yl)-2-fluorovinyl)-3-(2-fluorophenoxy)-2-(trifluoromethyl)phenyl)-9-ethyl-2,9-diazaspiro[5.5]undecane;
(Z)-2-(2-(6-(2-fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecan-9-yl)ethan-1-ol;
(Z)-4-(2-(2-(2-(9-ethyl-2,9-diazaspiro[5.5]undecan-2-yl)-4-(2-fluorophenoxy)-3-(trifluoromethyl)phenyl)-1-fluorovinyl)pyrimidin-4-yl)isothiazole;
(Z)-2-(3-(cyclohexyloxy)-6-(2-fluoro-2-(6-(pyridazin-4-yl)pyrazin-2-yl)vinyl)-2-(trifluoromethyl)phenyl)-9-(cyclopropylmethyl)-2,9-diazaspiro[5.5]undecane;
(Z)-9-(cyclopropylmethyl)-2-(6-(2-(6-(3,3-difluoroazetidin-1-yl)pyrazin-2-yl)-2-fluorovinyl)-3-(2-fluorophenoxy)-2-(trifluoromethyl)phenyl)-2,9-diazaspiro[5.5]undecane;
(Z)-2-(6-(2-(6-(3,3-difluoropyrrolidin-1-yl)pyrazin-2-yl)-2-fluorovinyl)-3-(2-fluorophenoxy)-2-(trifluoromethyl)phenyl)-9-ethyl-2,9-diazaspiro[5.5]undecane; and
(Z)-9-(cyclopropylmethyl)-2-(6-(2-(6-(3,3-difluoropyrrolidin-1-yl)pyrazin-2-yl)-2-fluorovinyl)-3-(2-fluorophenoxy)-2-(trifluoromethyl)phenyl)-2,9-diazaspiro[5.5]undecane;
   or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the terms “Cₙ₋ₘ” and “Cₘ₋ₙ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, from 2 to 6 carbon atoms, from 2 to 4 carbon atoms, from 2 to 3 carbon atoms, or 1 to 2 carbon atoms. The term “Cₙ₋ₘ alkyl” is understood to include deuterated analogs of alkyl groups as defined herein, including but not limited to, groups such as trideuteromethyl (CD₃), pentadeuteroethyl (CD₂CD₃), and the like.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms. The term “Cₙ₋ₘ alkenyl” is understood to include deuterated analogs of alkenyl groups as defined herein, including but not limited to, groups such as trideuteroethenyl (—CD═CD₂), tetradeuteropropenyl, (—CD═CD-CD₂), and the like.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms. The term “Cₙ₋ₘ alkynyl” is understood to include deuterated analogs of alkynyl groups as defined herein, including but not limited to, groups such as deuteroethynyl (—C≡CD), trideuteropropyn-1-yl, (—C≡CCD₃), and the like.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. The term “Cₙ₋ₘ alkoxy” is understood to include deuterated analogs of the alkyl moiety of the alkoxy group as defined herein, including but not limited to, groups such as trideuteromethoxy (—OCD₃), pentadeuteroethoxy (—OCD₂CD₃), and the like.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, and the like. In some embodiments, aryl groups have from 5 to 10 carbon atoms. In some embodiments, the aryl group is phenyl or naphthyl. In some embodiments, the aryl is phenyl. The term “aryl” is understood to include deuterated analogs of the aryl groups as defined herein, including but not limited to, groups such as pentadeuterophenyl (i.e., perdeuterophenyl, phenyl-ds), perdeuteronaphthyl, and the like.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms. The term “Cₙ₋ₘ haloalkoxy” is understood to include deuterated analogs of the haloalkyl moiety of the haloalkoxy group as defined herein, including but not limited to, groups such as deuterodifluoromethoxy (—OCDF₂), dideuterofluoromethoxy (—OCD₂F), and the like.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like. The term “Cₙ₋ₘ haloalkyl” is understood to include deuterated analogs of the haloalkyl group as defined herein, including but not limited to, groups such as deuterodifluoromethyl (—CDF₂), dideuterofluoromethyl (—CD₂F), and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl.

In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl. The term “cycloalkyl” is understood to include deuterated analogs of the cycloalkyl groups as defined herein, including but not limited to, groups such as perdeuterocyclopropyl, perdeuterocyclobutyl, perdeuterocyclopentyl, perdeuterocyclohexyl, and the like.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-, 7-, 8-, 9-, 10-, 11-, 12-, 13-, 14- or 15-membered monocyclic or bicyclic heteroaryl having 1, 2, 3, or 4 heteroatom ring members independently selected from N, O, S, and B. In some embodiments, the heteroaryl is a 5-10, or 5-15, membered monocyclic or bicyclic heteroaryl having 1, 2, 3, or 4 heteroatom ring members independently selected from N, O, and S. In some embodiments, the heteroaryl is a 5-, 7-, 8-, 9-, or 10-membered monocyclic or bicyclic heteroaryl having 1, 2, 3, or 4 heteroatom ring members independently selected from N, O, and S. In some embodiments, the heteroaryl is a 5-6 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, and S. In some embodiments, the heteroaryl group contains 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, thiazolo[4,5-e][1,2,4]triazolo[4,3-a]pyrimidinyl, 1H-pyrazolo[4,3-e][1,2,4]triazolo[4,3-a]pyrimidinyl, furo[2,3-e][1,2,4]triazolo[4,3-a]pyrimidinyl, thieno[2,3-e][1,2,4]triazolo[4,3-a]pyrimidinyl, 1H-[1,2,4]triazolo[3,4-b]purinyl, 6H-pyrrolo[2,3-e][1,2,4]triazolo[4,3-a]pyrimidinyl, thieno[2,3-e][1,2,4]triazolo[4,3-a]pyridinyl, 1H-pyrazolo[4,3-e][1,2,4]triazolo[4,3-a]pyridinyl, and the like. The term “heteroaryl” is understood to include deuterated analogs of the heteroaryl groups as defined herein, including but not limited to, groups such as perdeuteropyridinyl, perdeuteropyrazinyl, perdeuteropyrimidinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 15, 3 to 10, 4 to 10, 4 to 15, 5 to 10, 4 to 7, 5 to 7, or 5 to 6 membered heterocycloalkyl groups. Heterocycloalkyl groups can also include spirocycles and bridged rings (e.g., a 5 to 10, or 4 to 15, membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. The term “heterocycloalkyl” is understood to include deuterated analogs of the heterocycloalkyl groups as defined herein, including but not limited to, groups such as perdeuteroazetidinyl, perdeuteropyrrolidinyl, perdeuteropiperidinyl, and the like.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 15 ring-forming atoms, 4 to 10 ring-forming atoms, 4 to 8 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10, or 5-15, membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, tetrahydrothiopheneyl, tetrahydrothiopheneyl 1,1-dioxide, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, 2-azaspiro[3.3]heptanyl, diazaspiro[3.3]heptanyl, azaspiro[3.5]nonanyl, 7-azaspiro[3.5]nonanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl, diazaspiro[5.5]undecanyl, diazaspiro[5.6]dodecanyl, diazaspiro[6.6]tridecanyl, oxa-diazaspiro[5.5]undecanyl, 1-oxa-4,9-diazaspiro[5.5]undecanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms. The term “Cₒ₋ₚ cycloalkyl-Cₙ₋ₘ alkyl-” is understood to include deuterated analogs of the cycloalkyl and/or alkyl moieties of the Cₒ₋ₚ cycloalkyl-Cₙ₋ₘ alkyl- groups as defined herein.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms. The term “Cₒ₋ₚ aryl-Cₙ₋ₘ alkyl-” is understood to include deuterated analogs of the aryl and/or alkyl moieties of the Cₒ₋ₚ aryl-Cₙ₋ₘ alkyl- groups as defined herein.

As used herein, “heteroaryl-Cₙ₋ₘ alkyl-” refers to a group of formula heteroaryl-alkylene-, wherein alkylene linking group has n to m carbon atoms. The term “heteroaryl-Cₙ₋ₘ alkyl-” is understood to include deuterated analogs of the heteroaryl and/or alkyl moieties of the heteroaryl-Cₙ₋ₘ alkyl- groups as defined herein.

As used herein “heterocycloalkyl-Cₙ₋ₘ alkyl-” refers to a group of formula heterocycloalkyl-alkylene-, wherein alkylene linking group has n to m carbon atoms. The term “heterocycloalkyl-Cₙ₋ₘ alkyl-” is understood to include deuterated analogs of the heterocycloalkyl and/or alkyl moieties of the heterocycloalkyl-Cₙ₋ₘ alkyl- groups as defined herein.

As used herein, an “alkyl linking group” or “alkylene linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like. The terms “alkyl linking group” and “alkylene linking group” are understood to include deuterated analogs of the alkylene groups as defined herein.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as 0-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula I can be synthesized, for example, using the process shown in Scheme 1. As depicted in Scheme 1, nucleophilic aromatic substitution reactions of compounds of Formula 1-1 (e.g., wherein the Halo² group is a fluorine and the Halo¹ group is a chloro, bromo, or iodo) under appropriate conditions (e.g., in the presence of base, such as Cs₂CO₃) generates compounds of Formula 1-2. Alternatively, transition-metal (e.g., Pd, Cu, Ni) catalyzed coupling reactions (including, but not limited to, Buchwald-Hartwig, Ullman, Suzuki, Stille, and Negishi couplings) between compounds of Formula 1-1 (e.g., wherein the Halo² group is a chloro, bromo, or iodo) and appropriate coupling partners generates compounds of Formula 1-2. Compounds of Formula 1-1 are commercially available, or can be readily synthesized according to methods known by persons skilled in the art. Nucleophilic aromatic substitution reactions of compounds of Formula 1-2 with appropriate amine nucleophiles under appropriate conditions (e.g., in the presence of a base, such as N-ethyl-N-isopropylpropan-2-amine, in an appropriate solvent, such as CH₃CN) affords compounds of Formula 1-3.

Alternatively, transition metal catalyzed couplings (including, but not limited to, Buchwald-Hartwig couplings) between compounds of Formula 1-2 and appropriately substituted amines affords compounds of Formula 1-3. Reduction of the nitro group to compounds of Formula 1-3 under suitable conditions (e.g., using iron as reductant in the presence of an additive, such as NH₄Cl, in an appropriate solvent mixture, such as THF/MeOH/H₂O), followed by conversion to an iodide (e.g., via Sandmeyer reaction) provides compounds of Formula 1-4. Suzuki coupling of the iodide group in compounds of Formula 1-4 with an appropriate 2,2-difluorovinyl boronic ester (i.e., 2-(2,2-difluorovinyl)-4,4,5,5-tetramethyl-1,3,2-dioxaborolane) under appropriate conditions (e.g., in the presence of a palladium catalyst, such as [1,1′-bis(diphenylphosphino)ferrocene]dichloropalladium (II), and a base, such as Cs₂CO₃) provides compounds of Formula 1-5. Copper-catalyzed regioselective monodefluoroborylation of compounds of Formula 1-5 under appropriate conditions (e.g., such as described in J. Am. Chem. Soc. 2017, 139, 12855-12862) provides compounds of Formula 1-6. Finally, compounds of Formula I can be synthesized by transition metal catalyzed coupling reactions (including, but not limited to, Suzuki, Chan-Lam couplings) of compounds of Formula 1-6 with an appropriately substituted heteroaromatic coupling partner.

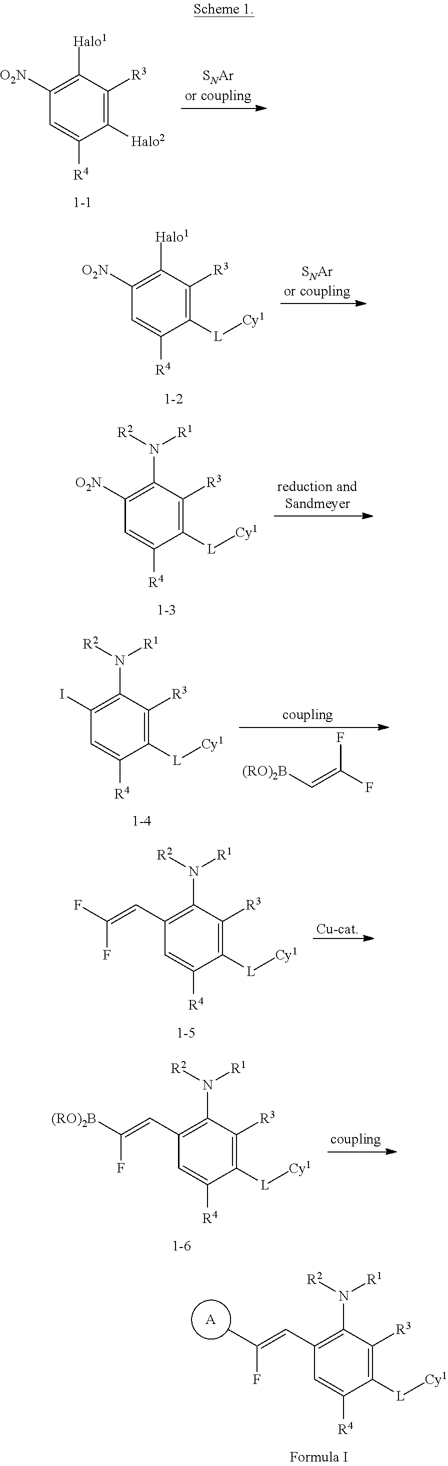

Alternatively, compounds of Formula 1-3 can be synthesized, for example, using the process shown in Scheme 2. As depicted in Scheme 2, nucleophilic aromatic substitution reactions of compounds of Formula 2-1 (e.g., wherein the Halo¹ group is a fluorine and the Halo² group is a chloro, bromo, or iodo) under appropriate conditions (e.g., in the presence of a base, such as N-ethyl-N-isopropylpropan-2-amine) generates compounds of Formula 2-2. Alternatively, transition metal catalyzed couplings (including, but not limited to, Buchwald-Hartwig couplings) between compounds of Formula 2-1 (e.g., wherein the Halo¹ group is a chloro, bromo, or iodo) and appropriately substituted amines affords compounds of Formula 2-2. Compounds of Formula 2-1 are commercially available, or can be readily synthesized according to methods known by persons skilled in the art. Nucleophilic aromatic substitution reactions of compounds of Formula 2-2 with appropriate amine nucleophiles under appropriate conditions (e.g., in the presence of base, such as Cs₂CO₃, in an appropriate solvent, such as CH₃CN) affords compounds of Formula 1-3. Alternatively, transition-metal (e.g., Pd, Cu, Ni) catalyzed coupling reactions (including, but not limited to, Buchwald-Hartwig, Ullman, Suzuki, Stille, and Negishi couplings) between compounds of Formula 2-2 and appropriate coupling partners under suitable conditions generates compounds of Formula 1-3. Compounds of Formula I can be synthesized from compounds of Formula 1-3 via the process shown in Scheme 1.

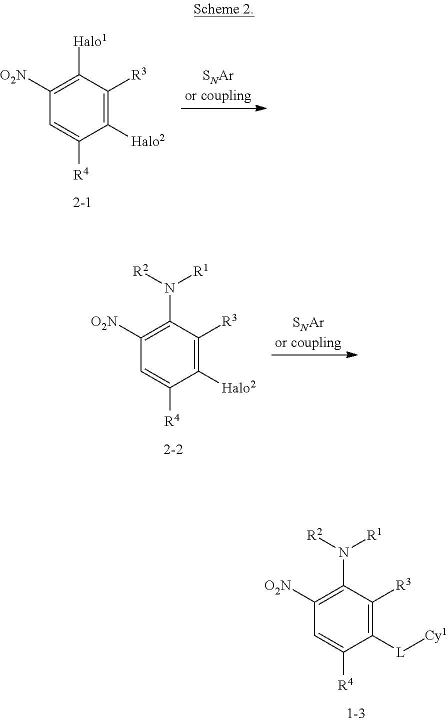

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature”, or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can inhibit the activity of DGK. Compounds that inhibit DGK are useful in providing a means of preventing the growth or inducing apoptosis of cancer cells. Such compounds are also useful in treating cancer cells exhibiting alterations in diacylglyceraol-regulating enzymes and effectors. It is therefore anticipated that the compounds of the disclosure are useful in treating or preventing cancer, such as solid tumors.

In certain embodiments, the disclosure provides a method for treating a DGK-related disorder in a patient in need thereof, comprising the step of administering to said patient a compound of the disclosure, or a pharmaceutically acceptable composition thereof.

The compounds or salts described herein can be selective. By “selective,” it is meant that the compound binds to or inhibits DGKα or DGKζ with greater affinity or potency, respectively, compared to at least one other DGK isoforms, or kinase, etc. In some embodiments, selectivity can be at least about 2-fold, 5-fold, 10-fold, at least about 20-fold, at least about 50-fold, at least about 100-fold, at least about 200-fold, at least about 500-fold or at least about 1000-fold. The compounds of the present disclosure can also be dual antagonists (i.e., inhibitors), e.g. inhibit both DGKα and DGKζ kinases. In some embodiments, the compounds of the invention are selective inhibitors of DGKα (e.g., over one or more other DGK isoforms, or kinase, etc.). In some embodiments, the compounds of the invention are selective inhibitors of DGKζ (e.g., over one or more other DGK isoforms, or kinase, etc.). Selectivity can be measured by methods routine in the art. In some embodiments, selectivity can be tested at the Kₘ ATP concentration of each enzyme. In some embodiments, the selectivity of compounds of the invention can be determined by cellular assays associated with particular DGK kinase activity.

Based on compelling evidence that DGKα and DGK negatively regulate signaling pathways downstream of the T cell receptor, developing DGK inhibitors can boost T cell effector function and inhibit tumor progression. DGK inhibitors can be used to treat, alone or in combination with other therapies, renal cell carcinoma, mesothelioma, glioblastoma multiforme, colorectal cancer, melanoma, pancreatic cancer (Chen, S. S. et al., Front. Cell Dev. Biol., 2016. 4:130; Gu, J. et al., Oncoimmunol., 2021. 10, e1941566; Jung I.-Y. et al., Cancer Res., 2018. 78:p4692-4703; Sitaram, P., et al., Int. J Mol. Sci., 2019. 20:p5821-5848; Wesley, E. M., et al., Immunohorizons, 2018. 2:p107-118)

In addition, DGKα has been shown to enhance esophageal squamous cell carcinoma (ESCC), and human hepatocellular carcinoma (HCC) progression (Chen, J. et al., Oncogene, 2019. 38: p2533-2550; Takeishi, K. et al., J. Hepatol., 2012. 57:p77-83), to support colon and breast cancer growth in three-dimensional (3D) culture (Torres-Ayuso, P. et al., Oncotarget, 2014. 5:p9710-9726), to enhance mammary carcinoma invasiveness (Rainero, E. et al., PLOS ONE, 2014. 9(6): e97144) and promote metastasis of non-small cell lung cancer (NSCLC) (Fu, L. et al., Cancer letters, 2022. 532: 215585) whereas DGKζ has been implicated as a potential oncogene in osteosarcoma proliferation (Yu, W. et al., Front. Oncol., 2019. 8:655) and contributed to enhanced invasion of human metastatic colon cancer cells (Cai, K. et al., BMC Cancer, 2014. 14:208). It has also been reported DGK inhibition has the potential to reduce immunopathology in X-linked lymphoproliferative disease patient (Velnati, S. et al., Eur. J. Med. Chem., 2019. 164: p378-390; Ruffo, E. et al., Sci. Transl. Med. 2016. 8 (321):321ra7).

In some embodiments, the DGK-related disorder is a solid tumor. Example solid tumors include, but are not limited to, breast cancer, colorectal cancer, gastric cancer, and glioblastoma (see e.g., Cooke & Kazanietz, Sci. Signal, 2022, 15, eabo0264:1-26). Example cancers associated with alterations in DAG-regulating enzymes and effector include, but are not limited to, uveal melanoma, myelodysplastic syndrome (MDS), angiosarcoma, nodal peripheral T cell lymphoma, adult T-cell leukemia lymphoma (ATLL), cutaneous T-cell lymphoma (CTCL)/Sezary syndrome, chronic lymphocytic leukemia (CLL), breast cancer, gastric cancer, colorectal cancer, oral squamous cell carcinoma (SCC), esophageal SCC, chronic myeloid leukemia (CML), colon cancer, prostate cancer, hepatocellular carcinoma (HCC), blue nevi, NK/T cell lymphoma, glioma, ovarian cancer, liver cancer, melanoma, heptacarcinoma, ostersarcoma, chordiod glioma, pigmented epithelioid melanocytoma, papillary glioneuronal tumor, fibrous histiocytoma, pituitary tumor, thyroid cancer, head and neck SCC, lung cancer, pediatric T-cell acute lymphoblastic leukemia (T-ALL), endometrial cancer, angiolipoma, salivary gland cancer, acute myeloid leukemia (AML), Epstein-Barr virus-associated (EBV)-associated B cell lymphoma, diffuse large B cell lymphoma (DLBCL), and cervical cancer (see e.g., Cooke & Kazanietz, Sci. Signal, 2022, 15, eabo0264:1-26).

In some embodiments, the cancer is selected from lung cancer, bladder cancer, urothelial cancer, esophageal cancer, stomach cancer, mesothelioma, liver cancer, diffuse large B cell lymphoma, kidney cancer, head and neck cancer, cholangiocarcinoma, cervical cancer, endocervical cancer, and melanoma.

In some embodiments, the cancer is selected from non-small cell lung cancer (lung squamous cell carcinoma (LUSC), lung adenocarcinoma (LUAD)), bladder urothelial carcinoma, esophageal carcinoma, stomach adenocarcinoma, mesothelioma, liver hepatocellular carcinoma, diffuse large B cell lymphoma (DLBCL), kidney renal clear cell carcinoma, head and neck squamous cell carcinoma, cholangiocarcinoma, cervical squamous cell carcinoma, endocervical adenocarcinoma, and metastatic melanoma.

In some embodiments, the cancer is a myelodysplastic syndrome. As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥10% of 1 cell |
| unilineage dysplasia | | line, <5% blasts |
| (RCUD) | | |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × | Dysplasia in ≥ 10% of cells in ≥2 |
| multilineage dysplasia | 10⁹/L monocytes | hematopoietic lineages, ±15% |
| | | ring sideroblasts, <5% blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% to |
| | monocytes | 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ±Auer rods, 10% to |
| | monocytes | 19% blasts |
| Myelodysplastic | Cytopenias | Unilineage or no dysplasia but |
| syndrome, unclassified | | characteristic MDS |
| (MDS-U) | | cytogenetics, <5% blasts |
| MDS associated with | Anemia, platelets | Unilineage erythroid. Isolated |
| isolated del(5q) | normal or increased | del(5q), <5% blasts |

In some embodiments, the myelodysplastic syndrome is refractory cytopenia with unilineage dysplasia (RCUD).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del(5q).

In some embodiments, the myelodysplastic syndrome is refractory to erythropoiesis-stimulating agents.

In some embodiments, the compounds of the disclosure can be useful in the treatment of myeloproliferative disorder/myelodysplastic overlap syndrome (MPD/MDS overlap syndrome).

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a DGK with a compound described herein includes the administration of a compound described herein to an individual or patient, such as a human, having a DGK, as well as, for example, introducing a compound described herein into a sample containing a cellular or purified preparation containing the DGK.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

### I. Immune-Checkpoint Therapies

In some embodiments, DGKα and DGKζ inhibitors provided herein can be used in combination with one or more immune checkpoint inhibitors for the treatment of cancer as described herein.

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1 BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, or 10,308,644; U.S. Publ. Nos. 2017/0145025, 2017/0174671, 2017/0174679, 2017/0320875, 2017/0342060, 2017/0362253, 2018/0016260, 2018/0057486, 2018/0177784, 2018/0177870, 2018/0179179, 2018/0179201, 2018/0179202, 2018/0273519, 2019/0040082, 2019/0062345, 2019/0071439, 2019/0127467, 2019/0144439, 2019/0202824, 2019/0225601, 2019/0300524, or 2019/0345170; or PCT Pub. Nos. WO 03042402, WO 2008156712, WO 2010089411, WO 2010036959, WO 2011066342, WO 2011159877, WO 2011082400, or WO 2011161699, which are each incorporated herein by reference in their entirety. In some embodiments, the inhibitor of PD-L1 is INCB086550.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012 (INCMGA0012; retifanlimab). In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGF.beta. receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound in a single or continuous dosage form, or they can be administered simultaneously or sequentially as separate dosage forms.

### II. Cancer Therapies

Cancer cell growth and survival can be impacted by multiple signaling pathways. Thus, it is useful to combine different enzyme/protein/receptor inhibitors, exhibiting different preferences in the targets which they modulate the activities of, to treat such conditions. Examples of agents that may be combined with compounds of the present disclosure, or solid forms or salts thereof, include inhibitors of the PI3K-AKT-mTOR pathway, inhibitors of the Raf-MAPK pathway, inhibitors of JAK-STAT pathway, inhibitors of beta catenin pathway, inhibitors of notch pathway, inhibitors of hedgehog pathway, inhibitors of Pim kinases, and inhibitors of protein chaperones and cell cycle progression. Targeting more than one signaling pathway (or more than one biological molecule involved in a given signaling pathway) may reduce the likelihood of drug-resistance arising in a cell population, and/or reduce the toxicity of treatment.

The compounds of the present disclosure, or solid forms or salts thereof, can be used in combination with one or more other enzyme/protein/receptor inhibitors for the treatment of diseases, such as cancer. Examples of cancers include solid tumors and liquid tumors, such as blood cancers. For example, the compounds of the present disclosure, or solid forms or salts thereof, can be combined with one or more inhibitors of the following kinases for the treatment of cancer: Akt1, Akt2, Akt3, TGF-□R, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure, or solid forms or salts thereof, can be combined with one or more of the following inhibitors for the treatment of cancer. Non-limiting examples of inhibitors that can be combined with the compounds of the present disclosure, or solid forms or salts thereof, for treatment of cancers include an FGFR inhibitor (FGFR1, FGFR2, FGFR3 or FGFR4, e.g., AZD4547, BAY1187982, ARQ087, BGJ398, BIBF1120, TKI258, lucitanib, dovitinib, TAS-120, JNJ-42756493, Debio1347, INCB54828, INCB62079 and INCB63904), a JAK inhibitor (JAK1 and/or JAK2, e.g., ruxolitinib, baricitinib or INCB39110), an IDO inhibitor (e.g., epacadostat and NLG919), an LSD1 inhibitor (e.g., GSK2979552, INCB59872 and INCB60003), a TDO inhibitor, a PI3K-delta inhibitor (e.g., INCB50797 and INCB50465), a PI3K-gamma inhibitor such as a PI3K-gamma selective inhibitor, a CSF1R inhibitor (e.g., PLX3397 and LY3022855), a TAM receptor tyrosine kinases (Tyro-3, Ax1, and Mer), an angiogenesis inhibitor, an interleukin receptor inhibitor, bromo and extra terminal family members inhibitors (for example, bromodomain inhibitors or BET inhibitors such as OTX015, CPI-0610, INCB54329 and INCB57643) and an adenosine receptor antagonist or combinations thereof. Inhibitors of HDAC such as panobinostat and vorinostat. Inhibitors of c-Met such as onartumzumab, tivantnib, and INC-280. Inhibitors of BTK such as ibrutinib. Inhibitors of mTOR such as rapamycin, sirolimus, temsirolimus, and everolimus. Inhibitors of Raf, such as vemurafenib and dabrafenib. Inhibitors of MEK such as trametinib, selumetinib and GDC-0973. Inhibitors of Hsp90 (e.g., tanespimycin), cyclin dependent kinases (e.g., palbociclib), PARP (e.g., olaparib) and Pim kinases (LGH447, INCB053914 and SGI-1776) can also be combined with compounds of the present disclosure.

Compounds of the present disclosure, or solid forms or salts thereof, can be used in combination with one or more agents for the treatment of diseases such as cancer. In some embodiments, the agent is an alkylating agent, a proteasome inhibitor, a corticosteroid, or an immunomodulatory agent. Examples of an alkylating agent include bendamustine, nitrogen mustards, ethylenimine derivatives, alkyl sulfonates, nitrosoureas and triazenes, uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

The compounds of the present disclosure, or solid forms or salts thereof, can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, adoptive T cell transfer, CAR (Chimeric antigen receptor) T cell treatment as a booster for T cell activation, oncolytic virotherapy and immunomodulating small molecules, including thalidomide or JAK1/2 inhibitor and the like. The compounds can be administered in combination with one or more anti-cancer drugs, such as a chemotherapeutics. Example chemotherapeutics include any of: abarelix, abiraterone, afatinib, aflibercept, aldesleukin, alemtuzumab, alitretinoin, allopurinol, altretamine, amsacrine, anastrozole, aphidicolon, arsenic trioxide, asparaginase, axitinib, azacitidine, bevacizumab, bexarotene, baricitinib, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, nofetumomab, oserelin, oxaliplatin, paclitaxel, pamidronate, panitumumab, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, ruxolitinib, sorafenib, streptozocin, sunitinib, sunitinib maleate, tamoxifen, tegafur, temozolomide, teniposide, testolactone, thalidomide, thioguanine, thiotepa, topotecan, toremifene, tositumomab, trastuzumab, tretinoin, triptorelin, uracil mustard, valrubicin, vandetanib, vinblastine, vincristine, vindesine, vinorelbine, vorinostat and zoledronate.

Other anti-cancer agent(s) include antibody therapeutics such as trastuzumab (Herceptin), antibodies to costimulatory molecules such as CTLA-4 (e.g., ipilimumab or tremelimumab), 4-1BB, antibodies to PD-1 and PD-L1, or antibodies to cytokines (IL-10, TGF-β, etc.). Examples of antibodies to PD-1 and/or PD-L1 that can be combined with compounds of the present disclosure for the treatment of cancer or infections such as viral, bacteria, fungus and parasite infections include, but are not limited to, nivolumab, pembrolizumab, MPDL3280A, MEDI-4736 and SHR-1210.

Other anti-cancer agents include inhibitors of kinases associated cell proliferative disorder. These kinases include but not limited to Aurora-A, CDK1, CDK2, CDK3, CDK5, CDK7, CDK8, CDK9, ephrin receptor kinases, CHK1, CHK2, SRC, Yes, Fyn, Lck, Fer, Fes, Syk, Itk, Bmx, GSK3, JNK, PAK1, PAK2, PAK3, PAK4, PDK1, PKA, PKC, Rsk, and SGK.

Other anti-cancer agents also include those that block immune cell migration such as antagonists to chemokine receptors, including CCR2 and CCR4.

The compounds of the present disclosure, or solid forms or salts thereof, can further be used in combination with one or more anti-inflammatory agents, steroids, immunosuppressants or therapeutic antibodies. The steroids include but are not limited to 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

The compounds of the present disclosure, or solid forms or salts thereof, can also be used in combination with lonafarnib (SCH6636), tipifarnib (R115777), L778123, BMS 214662, tezacitabine (MDL 101731), Sml1, triapine, didox, trimidox and amidox.

The compounds of the disclosure, or salts or solid forms thereof, can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure, or solid forms or salts thereof, can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure, or solid forms or salts thereof, can be used in combination with tumor specific antigen such as heat shock proteins isolated from tumor tissue itself. In some embodiments, the compounds of the present disclosure, or solid forms or salts thereof, can be combined with dendritic cells immunization to activate potent anti-tumor responses.

The compounds of the present disclosure, or solid forms or salts thereof, can be used in combination with bispecific macrocyclic peptides that target Fe alpha or Fe gamma receptor-expressing effectors cells to tumor cells. The compounds of the present disclosure, or solid forms or salts thereof, can also be combined with macrocyclic peptides that activate host immune responsiveness.

The compounds of the present disclosure, or solid forms or salts thereof, can be used in combination with bone marrow transplant for the treatment of a variety of tumors of hematopoietic origin.

Suitable antiviral agents contemplated for use in combination with the compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA).

Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

When more than one pharmaceutical agent is administered to a patient, they can be administered simultaneously, separately, sequentially, or in combination (e.g., for more than two agents).

In some embodiments, the compounds of the present disclosure, or solid forms or salts thereof, can be used in combination with INCB086550.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating DGK in tissue samples, including human, and for identifying DGK inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes DGK assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, the compound provided herein (e.g., the compound of any of Formulas I-IVb), or a pharmaceutically acceptable salt thereof, comprises at least one deuterium atom.

In some embodiments, the compound provided herein (e.g., the compound of any of Formulas I-IVb), or a pharmaceutically acceptable salt thereof, comprises two or more deuterium atoms.

In some embodiments, the compound provided herein (e.g., the compound of any of Formulas I-IVb), or a pharmaceutically acceptable salt thereof, comprises three or more deuterium atoms.

In some embodiments, for a compound provided herein (e.g., the compound of any of Formulas I-IVb), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro DGK labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind DGK by monitoring its concentration variation when contacting with DGK, through tracking of the labeling. For example, a test compound (labeled) can be evaluated for its ability to reduce binding of another compound which is known to bind to DGK (i.e., standard compound). Accordingly, the ability of a test compound to compete with the standard compound for binding to DGK directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of DGK-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 19×100 mm, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)). For purifications using a 30×100 mm column, the flow rate was 60 mL/minute.

pH=10 purifications: Waters XBridge™ C₁₈ 5 μm, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)). For purifications using a 30×100 mm column, the flow rate was 60 mL/minute.

### Intermediate 1. 4-(6-Chloropyrazin-2-yl)pyridazine

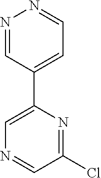 [SMILES: Clc1cncc(-c2ccnnc2)n1]

To a mixture of 2,6-dichloropyrazine (0.969 g, 6.50 mmol) in 1,4-dioxane (30 mL) was added copper(I) chloride (0.129 g, 1.300 mmol), lithium chloride (1.378 g, 32.5 mmol), tetrakis(triphenylphosphine)palladium(0) (0.751 g, 0.650 mmol) and 4-(tributylstannyl)pyridazine (2 mL, 6.50 mmol) and the reaction mixture was stirred at 120° C. for 6 hours. After cooling to rt, the reaction mixture was filtered through SiO₂, the filter cake was washed with CH₂Cl₂/MeOH, and the filtrate was concentrated in vacuo.

The crude residue was purified by flash column chromatography (SiO₂, CH₂Cl₂/MeOH) to afford the desired product (0.4 g, 32% yield) as yellow solid. LC-MS calculated for C₈H₆ClN₄ (M+H)⁺: m/z=193.0; found 193.0.

### Intermediate 2. 4-(6-Chloropyridin-2-yl)pyridazine

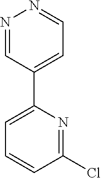 [SMILES: Clc1cccc(-c2ccnnc2)n1]

The title compound was prepared according to the procedures described in Intermediate 1, with 2,6-dichloropyridine replacing 2,6-dichloropyrazine. LC-MS calculated for C₉H₇ClN₃ (M+H)⁺: m/z=192.0; found 192.0.

### Intermediate 3. 4-(4-Chloropyrimidin-2-yl)pyridazine

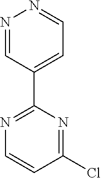 [SMILES: Clc1ccnc(-c2ccnnc2)n1]

The title compound was prepared according to the procedures described in Intermediate 1, with 4-chloro-2-iodopyrimidine replacing 2,6-dichloropyrazine. LC-MS calculated for C₈H₆ClN₄ (M+H)⁺: m/z=193.0; found 193.0.

### Intermediate 4. 4-(2-Iodopyrimidin-4-yl)pyridazine

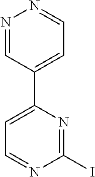 [SMILES: Ic1nccc(-c2ccnnc2)n1]

The title compound was prepared according to the procedures described in Intermediate 1, with 2,4-diiodopyrimidine replacing 2,6-dichloropyrazine. LC-MS calculated for C₈H₆IN₄ (M+H)⁺: m/z=285.0; found 285.0.

### Intermediate 5. 7-Chloro-5-(3,3-difluoroazetidin-1-yl)pyrido[3,4-b]pyrazine

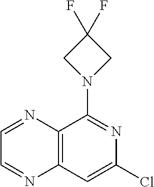 [SMILES: FC1(F)CN(c2nc(Cl)cc3nccnc23)C1]

To a mixture of 5,7-dichloropyrido[3,4-b]pyrazine (0.404 g, 2.020 mmol) in acetonitrile (10 mL) was added 3,3-difluoroazetidine hydrochloride (0.288 g, 2.222 mmol) and N-ethyl-N-isopropylpropan-2-amine (1.5 mL, 8.1 mmol) and the reaction mixture was stirred at 80° C. for 2 hours. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.25 g, 48% yield) as yellow solid. LC-MS calculated for C₁₀H₈ClF₂N₄ (M+H)⁺: m/z=257.0; found 257.0.

### Intermediate 6. 6-Chloro-8-(3,3-difluoroazetidin-1-yl)imidazo[1,2-a]pyrazine

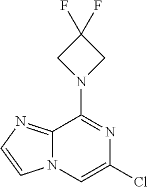 [SMILES: FC1(F)CN(c2nc(Cl)cn3ccnc23)C1]

The title compound was prepared according to the procedures described in Intermediate 5, with 6,8-dichloroimidazo[1,2-a]pyrazine replacing 5,7-dichloropyrido[3,4-b]pyrazine. LC-MS calculated for C₉H8ClF₂N₄ (M+H)⁺: m/z=245.0; found 245.0.

### Intermediate 7. 3-Chloro-5-(3,3-difluoroazetidin-1-yl)pyrazine-2-carbonitrile

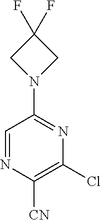 [SMILES: N#Cc1ncc(N2CC(F)(F)C2)nc1Cl]

The title compound was prepared according to a modification of the procedures described in Intermediate 5 with 3,5-dichloropyrazine-2-carbonitrile replacing 5,7-dichloropyrido[3,4-b]pyrazine. The reaction was conducted at rt. LC-MS calculated for C₈H₆ClF₂N₄ (M+H)⁺: m/z=231.0; found 230.9.

### Intermediate 8. 3-Chloro-5-(3,3-difluoropyrrolidin-1-yl)-1,2,4-triazine

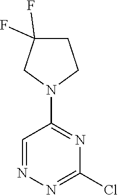 [SMILES: FC1(F)CCN(c2cnnc(Cl)n2)C1]

The title compound was prepared according to the procedures described in Intermediate 5, with 3,5-dichloro-1,2,4-triazine replacing 5,7-dichloropyrido[3,4-b]pyrazine and 3,3-difluoropyrrolidine replacing 3,3-difluoroazetidine hydrochloride. LC-MS calculated for C₇H8ClF₂N₄ (M+H)⁺: m/z=221.0; found 221.0.

### Intermediate 9. tert-Butyl (S,Z)-((1-(6-(2-fluoro-2-(4,4,5,5-tetramethyl-1,3,2-dioxaborolan-2-yl)vinyl)-3-(2-fluorophenoxy)-2-(trifluoromethyl)phenyl)piperidin-3-yl)methyl)carbamate

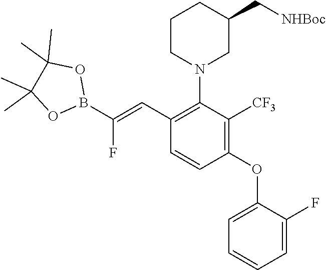 [SMILES: CC(C)(C)OC(=O)NC[C@@H]1CCCN(c2c(/C=C(\F)B3OC(C)(C)C(C)(C)O3)ccc(Oc3ccccc3F)c2C(F)(F)F)C1]

### Step 1. tert-Butyl (S)-((1-(3-(2-fluorophenoxy)-6-nitro-2-(trifluoromethyl)phenyl)piperidin-3-yl)methyl)carbamate

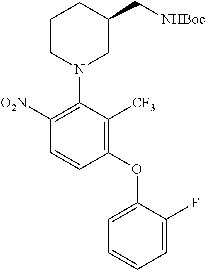 [SMILES: CC(C)(C)OC(=O)NC[C@@H]1CCCN(c2c([N+](=O)[O-])ccc(Oc3ccccc3F)c2C(F)(F)F)C1]

To a mixture of 2-bromo-4-fluoro-1-nitro-3-(trifluoromethyl)benzene (4.00 g, 13.9 mmol) in acetonitrile (15 mL) was added 2-fluorophenol (1.36 mL, 15.3 mmol) and cesium carbonate (9.05 g, 27.8 mmol), and the reaction mixture was stirred at 35° C. for 24 hours. After cooling to rt, tert-butyl (R)-(piperidin-3-ylmethyl)carbamate (3.27 g, 15.28 mmol) and N-ethyl-N-isopropylpropan-2-amine (5.13 mL, 27.8 mmol) were added and the reaction mixture was stirred at 80° C. for 3 hours. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (5.5 g, 77% yield) as a yellow oil. LC-MS calculated for C₂₄H₂₈F₄N₃O₅(M+H)⁺: m/z=514.2; found 514.2.

### Step 2. tert-Butyl (S)-((1-(6-amino-3-(2-fluorophenoxy)-2-(trifluoromethyl)phenyl)piperidin-3-yl)methyl)carbamate

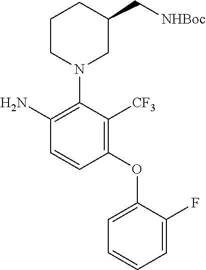 [SMILES: CC(C)(C)OC(=O)NC[C@@H]1CCCN(c2c(N)ccc(Oc3ccccc3F)c2C(F)(F)F)C1]

To a mixture of tert-butyl (S)-((1-(3-(2-fluorophenoxy)-6-nitro-2-(trifluoromethyl)phenyl)piperidin-3-yl)methyl)carbamate (1.00 g, 1.95 mmol) in THF/MeOH/H₂O (1:1:1) (12 mL) was added iron (0.326 g, 5.84 mmol) and ammonium chloride (0.521 g, 9.74 mmol) and the reaction mixture was stirred at 70° C. for 3 h. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.9 g, 96% yield) as a yellow solid. LC-MS calculated for C₂₄H₃₀F₄N₃O₃ (M+H)⁺: m/z=484.2; found 484.3.

### Step 3. tert-Butyl (S)-((1-(3-(2-fluorophenoxy)-6-iodo-2-(trifluoromethyl)phenyl)piperidin-3-yl)methyl)carbamate

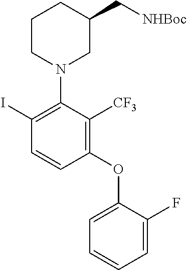 [SMILES: CC(C)(C)OC(=O)NC[C@@H]1CCCN(c2c(I)ccc(Oc3ccccc3F)c2C(F)(F)F)C1]

To a mixture of tert-butyl (S)-((1-(6-amino-3-(2-fluorophenoxy)-2-(trifluoromethyl)phenyl)piperidin-3-yl)methyl)carbamate (1.00 g, 2.07 mmol) in CH₂Cl₂/H₂O (10:1) (11 mL) was added sodium nitrite (0.713 g, 10.3 mmol), diiodomethane (0.50 mL, 6.2 mmol), and acetic acid (1.2 mL, 20 mmol) and the reaction mixture was stirred at rt for 1 hour. The reaction mixture was poured into a solution of sodium bicarbonate, and sodium thiosulfate. The mixture was extracted with EtOAc, dried over sodium sulfate, and concentrated. The crude residue was purified by flash column chromatography (SiO₂, EtOAc/Hexanes) to afford the desired product (0.56 g, 46% yield) as a white solid. LC-MS calculated for C₂₄H₂₈F₄₁N₂O₃(M+H)⁺: m/z=595.1; found 595.1.

### Step 4. tert-Butyl (S)-((1-(6-(2,2-difluorovinyl)-3-(2-fluorophenoxy)-2-(trifluoromethyl)phenyl)piperidin-3-yl)methyl)carbamate

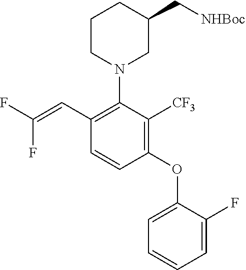 [SMILES: CC(C)(C)OC(=O)NC[C@@H]1CCCN(c2c(C=C(F)F)ccc(Oc3ccccc3F)c2C(F)(F)F)C1]

To a mixture of tert-butyl (S)-((1-(3-(2-fluorophenoxy)-6-iodo-2-(trifluoromethyl)phenyl)piperidin-3-yl)methyl)carbamate (0.50 g, 0.84 mmol) in 1,4-dioxane (8 mL) was added [1,1′-bis(diphenylphosphino)ferrocene]dichloropalladium(II) (0.123 g, 0.168 mmol), 2-(2,2-difluorovinyl)-4,4,5,5-tetramethyl-1,3,2-dioxaborolane (0.24 g, 1.3 mmol), cesium carbonate (0.822 g, 2.52 mmol) and the reaction mixture was stirred at 95° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.3 g, 67% yield) as a white waxy solid. LC-MS calculated for C₂₆H₂₉F₆N₂O₃(M+H)⁺: m/z=531.2; found 531.2.

### Step 5. tert-Butyl (S,Z)-((1-(6-(2-fluoro-2-(4,4,5,5-tetramethyl-1,3,2-dioxaborolan-2-yl)vinyl)-3-(2-fluorophenoxy)-2-(trifluoromethyl)phenyl)piperidin-3-yl)methyl)carbamate

To a mixture of tert-butyl (S)-((1-(6-(2,2-difluorovinyl)-3-(2-fluorophenoxy)-2-(trifluoromethyl)phenyl)piperidin-3-yl)methyl)carbamate (0.3 g, 0.565 mmol) in THF (5 mL) was added copper(I) chloride (0.017 g, 0.170 mmol), tricyclohexylphosphine (0.095 g, 0.339 mmol), 4,4,4′,4′,5,5,5′,5′-octamethyl-2,2′-bi(1,3,2-dioxaborolane) (0.431 g, 1.696 mmol), and potassium acetate (0.166 g, 1.696 mmol) and the reaction mixture was purged with N₂ and stirred at 40° C. overnight. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.32 g, 89% yield) as waxy solid. LC-MS calculated for C₂₆H₃₁BF₅N₂O₅ (M+H−C₆H₁₀)⁺: m/z=557.2; found 557.2.

### Intermediate 10. tert-Butyl (Z)-(3-((6-(2-fluoro-2-(4,4,5,5-tetramethyl-1,3,2-dioxaborolan-2-yl)vinyl)-3-phenoxy-2-(trifluoromethyl)phenyl)(methyl)amino)propyl)carbamate

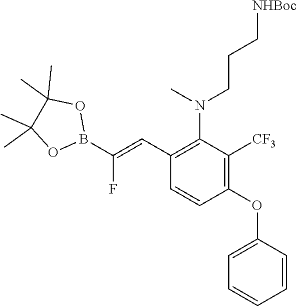 [SMILES: CN(CCCNC(=O)OC(C)(C)C)c1c(/C=C(\F)B2OC(C)(C)C(C)(C)O2)ccc(Oc2ccccc2)c1C(F)(F)F]

The title compound was prepared according to the procedures described in Intermediate 9, with phenol replacing 2-fluorophenol and tert-butyl (3-(methylamino)propyl)carbamate replacing tert-butyl (R)-(piperidin-3-ylmethyl)carbamate. LC-MS calculated for C₂₄H₂₉BF₄N₂O₅ (M+H−C₆H₁₀)⁺: m/z=513.2; found 513.2.

### Intermediate 11. tert-Butyl (Z)-4-(6-(2-fluoro-2-(4,4,5,5-tetramethyl-1,3,2-dioxaborolan-2-yl)vinyl)-3-phenoxy-2-(trifluoromethyl)phenyl)-1-oxa-4,9-diazaspiro[5.5]undecane-9-carboxylate

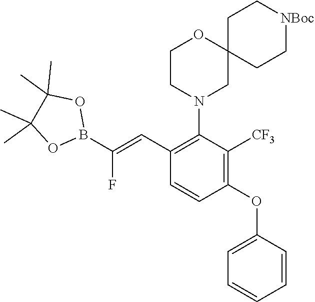 [SMILES: CC(C)(C)OC(=O)N1CCC2(CC1)CN(c1c(/C=C(\F)B3OC(C)(C)C(C)(C)O3)ccc(Oc3ccccc3)c1C(F)(F)F)CCO2]

The title compound was prepared according to the procedures described in Intermediate 9, with phenol replacing 2-fluorophenol and tert-butyl 1-oxa-4,9-diazaspiro[5.5]undecane-9-carboxylate replacing tert-butyl (R)-(piperidin-3-ylmethyl)carbamate. LC-MS calculated for C₂₈H₃₄BF₄N₂O₆ (M+H−C₆H₁₀)⁺: m/z=581.2; found 581.3.

### Intermediate 12. tert-Butyl (S,Z)-((1-(2-chloro-6-(2-fluoro-2-(4,4,5,5-tetramethyl-1,3,2-dioxaborolan-2-yl)vinyl)-3-(4-fluoro-2-methoxyphenoxy)phenyl)piperidin-3-yl)methyl)carbamate

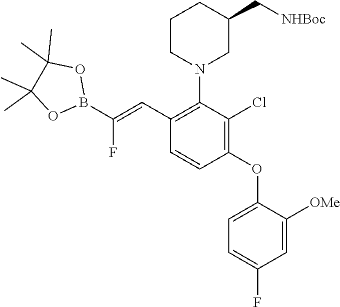 [SMILES: COc1cc(F)ccc1Oc1ccc(/C=C(\F)B2OC(C)(C)C(C)(C)O2)c(N2CCC[C@@H](CNC(=O)OC(C)(C)C)C2)c1Cl]

### Step 1. tert-Butyl (S)-((1-(3-bromo-2-chloro-6-nitrophenyl)piperidin-3-yl)methyl)carbamate

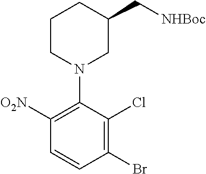 [SMILES: CC(C)(C)OC(=O)NC[C@@H]1CCCN(c2c([N+](=O)[O-])ccc(Br)c2Cl)C1]

To a mixture of 1-bromo-2-chloro-3-fluoro-4-nitrobenzene (2.5 g, 9.83 mmol) in acetonitrile (15 mL) was added tert-butyl (R)-(piperidin-3-ylmethyl)carbamate (2.316 g, 10.8 mmol) and N-ethyl-N-isopropylpropan-2-amine (5.15 mL, 29.5 mmol), and the reaction mixture was stirred at rt for 24 hours. The reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (3.6 g, 82% yield) as yellow oil. LC-MS calculated for C₁₇H₂₄BrClN₃O₄ (M+H)⁺: m/z=448.1; found 448.1.

### Step 2. tert-Butyl (S)-((1-(6-amino-2-chloro-3-(4-fluoro-2-methoxyphenoxy)phenyl)piperidin-3-yl)methyl)carbamate

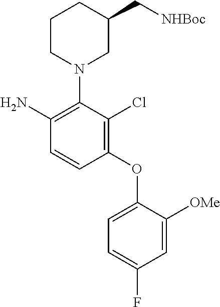 [SMILES: COc1cc(F)ccc1Oc1ccc(N)c(N2CCC[C@@H](CNC(=O)OC(C)(C)C)C2)c1Cl]

To a mixture of tert-butyl (S)-((1-(2-chloro-3-fluoro-6-nitrophenyl)piperidin-3-yl)methyl)carbamate (0.35 g, 0.90 mmol) in acetonitrile (15 mL) was added 4-fluoro-2-methoxyphenol (0.192 g, 1.35 mmol), cesium carbonate (0.882 g, 2.71 mmol), and the reaction mixture was stirred at 80° C. overnight. After cooling to rt, the reaction mixture was filtered through SiO₂, the filter cake was washed with EtOAc, and the filtrate was concentrated in vacuo. To a mixture of the crude residue in THF/MeOH/H₂O (1:1:1) (15 mL) was added iron (0.252 g, 4.51 mmol) and ammonium chloride (0.483 g, 9.0 mmol) and the reaction mixture was stirred at 70° C. for 3 hours. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.35 g, 81% yield) as brown solid. LC-MS calculated for C₂₄H₃₂ClFN₃O₄ (M+H)⁺: m/z=480.2; found 480.3.

### Step 3. tert-Butyl (S)-((1-(2-chloro-3-(4-fluoro-2-methoxyphenoxy)-6-iodophenyl)piperidin-3-yl)methyl)carbamate

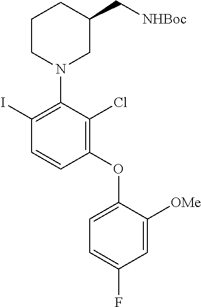 [SMILES: COc1cc(F)ccc1Oc1ccc(I)c(N2CCC[C@@H](CNC(=O)OC(C)(C)C)C2)c1Cl]

A mixture of tert-butyl (S)-((1-(6-amino-2-chloro-3-(4-fluoro-2-methoxyphenoxy)phenyl)piperidin-3-yl)methyl)carbamate (0.35 g, 0.73 mmol) in acetonitrile (5 mL) was cooled to 0° C. for 5 mins before 4-methylbenzenesulfonic acid (0.628 g, 3.65 mmol) was added, followed by a mixture of potassium iodide (0.605 g, 3.65 mmol) in water (1 mL) and a mixture of sodium nitrite (0.252 g, 3.65 mmol) in water (1 mL). The reaction mixture was warmed to rt and stirred for 1 hour. The reaction mixture was poured into a mixture of sodium bicarbonate and sodium thiosulfate, and the mixture was extracted with EtOAc. The combined organic layers were dried over sodium sulfate and concentrated in vacuo. The crude residue was purified by flash column chromatography (SiO₂, EtOAc/Hexanes) to afford the desired product (0.25 g, 58% yield) as white solid. LC-MS calculated for C₂₄H₃₀ClFIN₂O₄ (M+H)⁺: m/z=591.1; found 591.2.

### Step 4. tert-Butyl (S)-((1-(2-chloro-6-(2,2-difluorovinyl)-3-(4-fluoro-2-methoxyphenoxy)phenyl)piperidin-3-yl)methyl)carbamate

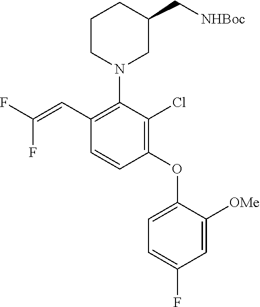 [SMILES: COc1cc(F)ccc1Oc1ccc(C=C(F)F)c(N2CCC[C@@H](CNC(=O)OC(C)(C)C)C2)c1Cl]

To a mixture of tert-butyl (S)-((1-(2-chloro-3-(4-fluoro-2-methoxyphenoxy)-6-iodophenyl)piperidin-3-yl)methyl)carbamate (0.25 g, 0.423 mmol) in 1,4-dioxane (5 mL) was added [1,1′-bis(diphenylphosphino)ferrocene]dichloropalladium(II) (0.046 g, 0.063 mmol), 2-(2,2-difluorovinyl)-4,4,5,5-tetramethyl-1,3,2-dioxaborolane (0.12 g, 0.635 mmol), cesium carbonate (0.42 g, 1.289 mmol) and the reaction mixture was stirred at 95° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.18 g, 81% yield) as a white waxy solid. LC-MS calculated for C₂₆H₃₁ClF₃N₂O₄ (M+H)⁺: m/z=527.2; found 527.2.

### Step 5. tert-Butyl (S,Z)-((1-(2-chloro-6-(2-fluoro-2-(4,4,5,5-tetramethyl-1,3,2-dioxaborolan-2-yl)vinyl)-3-(4-fluoro-2-methoxyphenoxy)phenyl)piperidin-3-yl)methyl)carbamate

To a mixture of tert-butyl (S)-((1-(2-chloro-6-(2,2-difluorovinyl)-3-(4-fluoro-2-methoxyphenoxy)phenyl)piperidin-3-yl)methyl)carbamate (0.18 g, 0.342 mmol) in THF (5 mL) was added copper(I) chloride (10.1 mg, 0.102 mmol), tricyclohexylphosphine (0.057 g, 0.205 mmol), 4,4,4′,4′,5,5,5′,5′-octamethyl-2,2′-bi(1,3,2-dioxaborolane) (0.260 g, 1.025 mmol), and potassium acetate (0.10 g, 1.025 mmol) and the reaction mixture was purged with N₂ and stirred at 40° C. overnight. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.15 g, 69% yield) as waxy solid. LC-MS calculated for C₂₆H₃₃BClF₂N₂O₆ (M+H−C₆H₁₀)⁺: m/z=553.2; found 553.3.

### Intermediate 13. tert-Butyl (R,Z)-((1-(2-chloro-6-(2-fluoro-2-(4,4,5,5-tetramethyl-1,3,2-dioxaborolan-2-yl)vinyl)-3-phenoxyphenyl)pyrrolidin-2-yl)methyl)carbamate

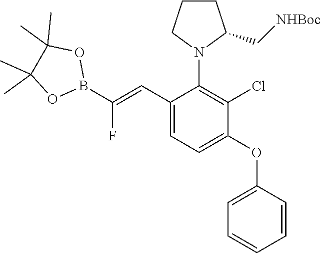 [SMILES: CC(C)(C)OC(=O)NC[C@H]1CCCN1c1c(/C=C(\F)B2OC(C)(C)C(C)(C)O2)ccc(Oc2ccccc2)c1Cl]

The title compound was prepared according to the procedures described in Intermediate 12, with phenol replacing 4-fluoro-2-methoxyphenol and tert-butyl (R)-(pyrrolidin-2-ylmethyl)carbamate replacing tert-butyl (R)-(piperidin-3-ylmethyl)carbamate. LC-MS calculated for C₂₄H₃₀BClFN₂O₅(M+H−C₆H₁₀)⁺: m/z=491.2; found 491.2.

### Intermediate 14. tert-Butyl 4-(3-cyclopropoxy-6-nitro-2-(trifluoromethyl)phenyl)-1-oxa-4,9-diazaspiro[5.5]undecane-9-carboxylate

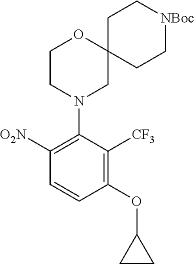 [SMILES: CC(C)(C)OC(=O)N1CCC2(CC1)CN(c1c([N+](=O)[O-])ccc(OC3CC3)c1C(F)(F)F)CCO2]

A mixture of cyclopropanol (0.200 mL, 3.15 mmol) in DMF (6.0 mL) was cooled to 0° C. before sodium hydride (144 mg, 3.60 mmol) was added in one portion. The reaction mixture was warmed to rt and stirred for 30 mins before 2-bromo-4-fluoro-1-nitro-3-(trifluoromethyl)benzene (0.864 g, 3.00 mmol) was added and the reaction mixture was stirred at 40° C. overnight. After cooling to rt, the reaction mixture was quenched by slow addition of water, extracted with EtOAc and washed with brine. The organic layer was dried over sodium sulfate and concentrated in vacuo. To a mixture of crude residue in acetonitrile (6.0 mL) was added tert-butyl 1-oxa-4,9-diazaspiro[5.5]undecane-9-carboxylate (0.769 g, 3 mmol) and N-ethyl-N-isopropylpropan-2-amine (0.78 mL, 4.5 mmol) and the reaction mixture was stirred at 80° C. for 3 hours. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.80 g, 53% yield) as a yellow oil. LC-MS calculated for C₁₉H₂₃F₃N₃O₆ (M+H−C₄H₈)⁺: m/z=446.2; found 446.1.

### Intermediate 15. tert-Butyl (Z)-4-(3-cyclopropoxy-6-(2-fluoro-2-(4,4,5,5-tetramethyl-1,3,2-dioxaborolan-2-yl)vinyl)-2-(trifluoromethyl)phenyl)-1-oxa-4,9-diazaspiro[5.5]undecane-9-carboxylate

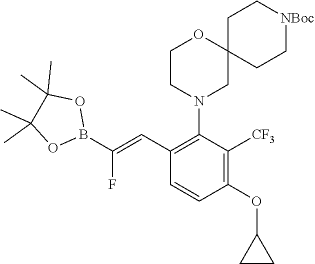 [SMILES: CC(C)(C)OC(=O)N1CCC2(CC1)CN(c1c(/C=C(\F)B3OC(C)(C)C(C)(C)O3)ccc(OC3CC3)c1C(F)(F)F)CCO2]

The title compound was prepared according to the procedures described in Intermediate 9, Steps 2-5 with tert-butyl 4-(3-cyclopropoxy-6-nitro-2-(trifluoromethyl)phenyl)-1-oxa-4,9-diazaspiro[5.5]undecane-9-carboxylate (Intermediate 14) replacing tert-butyl (S)-((1-(3-(2-fluorophenoxy)-6-nitro-2-(trifluoromethyl)phenyl)piperidin-3-yl)methyl)carbamate in Step 2. LC-MS calculated for C₂₅H₃₄BF₄N₂O₆ (M+H−C₆H₁₀)⁺: m/z=545.2; found 545.3.

### Intermediate 16. 4-(2-Bromopyrimidin-4-yl)pyridazine

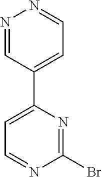 [SMILES: Brc1nccc(-c2ccnnc2)n1]

The title compound was prepared according to the procedures described in Intermediate 1, with 2,4-dibromopyrimidine replacing 2,6-dichloropyrazine. LC-MS calculated for C₈H₆BrN₄ (M+H)⁺: m/z=237.0; found 237.0.

### Intermediate 17. 2-Bromo-6-(3,3-difluoroazetidin-1-yl)pyrazine

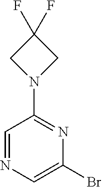 [SMILES: FC1(F)CN(c2cncc(Br)n2)C1]

The title compound was prepared according to the procedures described in Intermediate 5, with 2,6-dibromopyrazine replacing 5,7-dichloropyrido[3,4-b]pyrazine. LC-MS calculated for C₇H7BrF₂N₃(M+H)⁺: m/z=250.0; found 250.0.

### Intermediate 18. 2-Bromo-6-(3,3-difluoropyrrolidin-1-yl)pyrazine

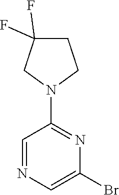 [SMILES: FC1(F)CCN(c2cncc(Br)n2)C1]

The title compound was prepared according to the procedures described in Intermediate 5, with 2,6-dibromopyrazine replacing 5,7-dichloropyrido[3,4-b]pyrazine and 3,3-difluoropyrrolidine replacing 3,3-difluoroazetidine hydrochloride. LC-MS calculated for C₈H₉BrF₂N₃(M+H)⁺: m/z=264.0; found 264.0.

### Intermediate 19. tert-Butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate

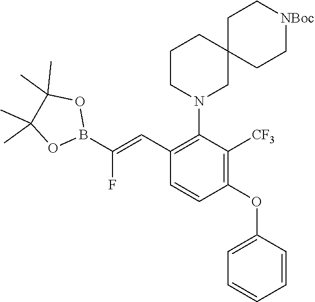 [SMILES: CC(C)(C)OC(=O)N1CCC2(CCCN(c3c(/C=C(\F)B4OC(C)(C)C(C)(C)O4)ccc(Oc4ccccc4)c3C(F)(F)F)C2)CC1]

### Step 1. tert-Butyl 2-(6-amino-3-phenoxy-2-(trifluoromethyl)phenyl)-2,9-diazaspiro[5.5]undecane-9-carboxylate

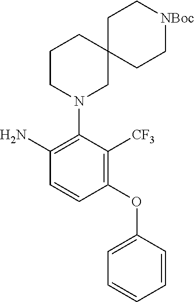 [SMILES: CC(C)(C)OC(=O)N1CCC2(CCCN(c3c(N)ccc(Oc4ccccc4)c3C(F)(F)F)C2)CC1]

To a mixture of 2-bromo-4-fluoro-1-nitro-3-(trifluoromethyl)benzene (5.00 g, 17.4 mmol) in acetonitrile (15 mL) was added phenol (1.80 g, 19.1 mmol) and cesium carbonate (11.3 g, 34.7 mmol), and the reaction mixture was stirred at 35° C. for 24 hours. After cooling to rt, the reaction mixture was filtered through a pad of silica gel, washed with Hexanes/EtOAc (1:1). The filtrate was concentrated to yield a yellow oil. To a mixture of the crude material in acetonitrile (20 mL) was added tert-butyl 2,9-diazaspiro[5.5]undecane-9-carboxylate (5.48 g, 21.5 mmol) and N-ethyl-N-isopropylpropan-2-amine (9.18 mL, 49.7 mmol) and the reaction mixture was stirred at 90° C. for 5 hours. After cooling to rt, hypodiboric acid (7.37 g, 82.0 mmol) was added to the reaction mixture followed by MeOH (20 mL). 4,4′-Bipyridine (0.128 g, 0.822 mmol) was added slowly and the reaction mixture was stirred at rt for 1 hour before the reaction mixture was poured into water. The mixture was extracted with EtOAc, dried over sodium sulfate, and concentrated in vacuo. The crude residue was purified by flash column chromatography (SiO₂, EtOAc/Hexanes) to afford the desired product (6.5 g, 78% yield) as a yellow oil. LC-MS calculated for C₂₇H₃₅F₃N₃O₃(M+H)⁺: m/z=506.3; found 506.3.

### Step 2. tert-Butyl 2-(6-iodo-3-phenoxy-2-(trifluoromethyl)phenyl)-2,9-diazaspiro[5.5]undecane-9-carboxylate

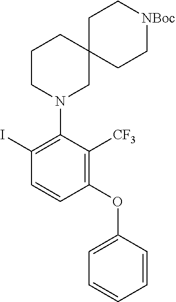 [SMILES: CC(C)(C)OC(=O)N1CCC2(CCCN(c3c(I)ccc(Oc4ccccc4)c3C(F)(F)F)C2)CC1]

To a mixture of tert-butyl 2-(6-amino-3-phenoxy-2-(trifluoromethyl)phenyl)-2,9-diazaspiro[5.5]undecane-9-carboxylate (6.00 g, 11.9 mmol) in ACN/H₂O (5:1, 200 mL) was added acetic acid (10.7 g, 178 mmol), potassium iodide (9.85 g, 59.3 mmol), and a solution of sodium nitrite (4.09 g, 59.3 mmol) in water (10 mL) and the reaction mixture was stirred at rt for 2 hours. The reaction mixture was poured into a saturated solution of sodium bicarbonate, and sodium thiosulfate. The mixture was extracted with EtOAc, dried over sodium sulfate, and concentrated in vacuo. The crude residue was purified by flash column chromatography (SiO₂, EtOAc/Hexanes) to afford the desired product (5 g, 68% yield) as a white waxy solid. LC-MS calculated for C₂₃H₂₅F₃IN₂O₃(M+H−C₄H₈)⁺: m/z=561.1; found 561.1

### Step 3. tert-Butyl 2-(6-(2,2-difluorovinyl)-3-phenoxy-2-(trifluoromethyl)phenyl)-2,9-diazaspiro[5.5]undecane-9-carboxylate

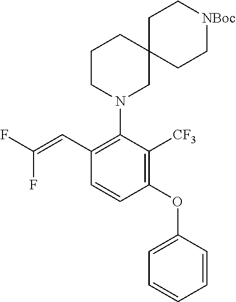 [SMILES: CC(C)(C)OC(=O)N1CCC2(CCCN(c3c(C=C(F)F)ccc(Oc4ccccc4)c3C(F)(F)F)C2)CC1]

To a mixture of tert-butyl 2-(6-iodo-3-phenoxy-2-(trifluoromethyl)phenyl)-2,9-diazaspiro[5.5]undecane-9-carboxylate (5.00 g, 8.11 mmol) in 1,4-dioxane/H₂O (4:1, 20 mL) was added [1,1′-bis(diphenylphosphino)ferrocene]dichloropalladium(II) (1.18 g, 16.2 mmol), 2-(2,2-difluorovinyl)-4,4,5,5-tetramethyl-1,3,2-dioxaborolane (3.10 g, 16.2 mmol), and cesium carbonate (7.90 g, 24.3 mmol) and the reaction mixture was stirred at 70° C. overnight. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (2.4 g, 54% yield) as a white waxy solid. LC-MS calculated for C₂₉H₃₄F₅N₂O₃(M+H)⁺: m/z=553.2; found 553.3.

### Step 4. tert-Butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate

To a mixture of tert-butyl 2-(6-(2,2-difluorovinyl)-3-phenoxy-2-(trifluoromethyl)phenyl)-2,9-diazaspiro[5.5]undecane-9-carboxylate (2.30 g, 4.16 mmol) in THF (20 mL) was added CuCl(PCy₃)₂(0.41 g, 0.62 mmol), 4,4,4′,4′,5,5,5′,5′-octamethyl-2,2′-bi(1,3,2-dioxaborolane) (3.17 g, 12.5 mmol), and potassium acetate (1.23 g, 12.5 mmol) and the reaction mixture was purged with N₂ and stirred at 40° C. overnight. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (2.1 g, 76% yield) as waxy solid. LC-MS calculated for C₂₉H₃₆BF₄N₂O₅ (M+H−C₆H₁₀)⁺: m/z=579.3; found 579.3. CuCl(PCy₃)₂ was prepared in accordance with literature procedures (see e.g., Bowmaker et al. J. Chem. Soc., Dalton Trans. 2002, 2722-2730).

### Intermediate 20. tert-Butyl (Z)-2-(6-(2-fluoro-2-(4,4,5,5-tetramethyl-1,3,2-dioxaborolan-2-yl)vinyl)-3-(phenoxy-d5)-2-(trifluoromethyl)phenyl)-2,9-diazaspiro[5.5]undecane-9-carboxylate

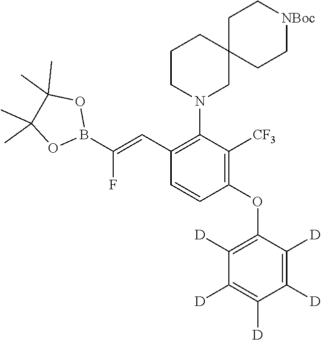 [SMILES: [2H]c1c([2H])c([2H])c(Oc2ccc(/C=C(\F)B3OC(C)(C)C(C)(C)O3)c(N3CCCC4(CCN(C(=O)OC(C)(C)C)CC4)C3)c2C(F)(F)F)c([2H])c1[2H]]

The title compound was prepared according to the procedures described in Intermediate 19, with phen-d₅-ol replacing phenol. LC-MS calculated for C₂₉H₃₁D₅BF₄N₂O₅ (M+H−C₆H₁₀)⁺: m/z=584.3; found 584.3.

### Intermediate 21. tert-Butyl (Z)-2-(6-(2-fluoro-2-(4,4,5,5-tetramethyl-1,3,2-dioxaborolan-2-yl)vinyl)-3-((3-fluoropyridin-2-yl)oxy)-2-(trifluoromethyl)phenyl)-2,9-diazaspiro[5.5]undecane-9-carboxylate

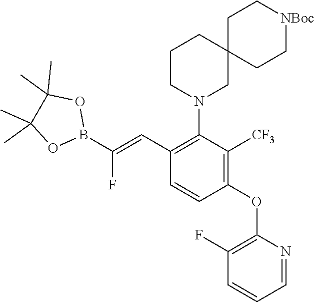 [SMILES: CC(C)(C)OC(=O)N1CCC2(CCCN(c3c(/C=C(\F)B4OC(C)(C)C(C)(C)O4)ccc(Oc4ncccc4F)c3C(F)(F)F)C2)CC1]

The title compound was prepared according to the procedures described in Intermediate 19, with 3-fluoropyridin-2-ol replacing phenol. LC-MS calculated for C₂₈H₃₄BF₅N₃O₅ (M+H−C₆H₁₀)⁺: m/z=598.2; found 598.3.

### Intermediate 22. tert-Butyl (Z)-4-(6-(2-fluoro-2-(4,4,5,5-tetramethyl-1,3,2-dioxaborolan-2-yl)vinyl)-3-(3-fluorophenoxy)-2-(trifluoromethyl)phenyl)-1-oxa-4,9-diazaspiro[5.5]undecane-9-carboxylate

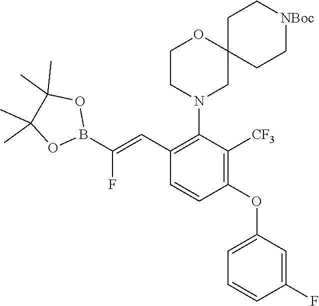 [SMILES: CC(C)(C)OC(=O)N1CCC2(CC1)CN(c1c(/C=C(\F)B3OC(C)(C)C(C)(C)O3)ccc(Oc3cccc(F)c3)c1C(F)(F)F)CCO2]

The title compound was prepared according to the procedures described in Intermediate 19, with 3-fluorophenol replacing phenol and tert-butyl 1-oxa-4,9-diazaspiro[5.5]undecane-9-carboxylate replacing tert-butyl 2,9-diazaspiro[5.5]undecane-9-carboxylate in step 1. LC-MS calculated for C₂₈H₃₃BF₅N₂O₆ (M+H−C₆H₁₀)⁺: m/z=599.2; found 599.3.

### Intermediate 23. tert-Butyl (Z)-4-(6-(2-fluoro-2-(4,4,5,5-tetramethyl-1,3,2-dioxaborolan-2-yl)vinyl)-3-(2-fluorophenoxy)-2-(trifluoromethyl)phenyl)-1-oxa-4,9-diazaspiro[5.5]undecane-9-carboxylate

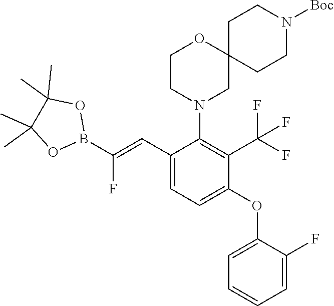 [SMILES: CC(C)(C)OC(=O)N1CCC2(CC1)CN(c1c(/C=C(\F)B3OC(C)(C)C(C)(C)O3)ccc(Oc3ccccc3F)c1C(F)(F)F)CCO2]

The title compound was prepared according to the procedures described in Intermediate 19, with 2-fluorophenol replacing phenol and tert-butyl 1-oxa-4,9-diazaspiro[5.5]undecane-9-carboxylate replacing tert-butyl 2,9-diazaspiro[5.5]undecane-9-carboxylate in step 1. LC-MS calculated for C₂₈H₃₃BF₅N₂O₆ (M+H−C₆H₁₀)⁺: m/z=599.2; found 599.3.

### Intermediate 24. tert-Butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate

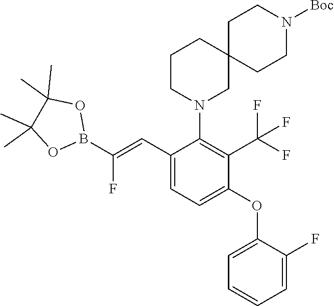 [SMILES: CC(C)(C)OC(=O)N1CCC2(CCCN(c3c(/C=C(\F)B4OC(C)(C)C(C)(C)O4)ccc(Oc4ccccc4F)c3C(F)(F)F)C2)CC1]

The title compound was prepared according to the procedures described in Intermediate 19, with 2-fluorophenol replacing phenol in step 1. LC-MS calculated for C₂₉H₃₅BF₅N₂O₅ (M+H−C₆H₁₀)⁺: m/z=597.3; found 597.2.

### Intermediate 25. tert-Butyl (Z)-2-(3-(2,4-difluorophenoxy)-6-(2-fluoro-2-(4,4,5,5-tetramethyl-1,3,2-dioxaborolan-2-yl)vinyl)-2-(trifluoromethyl)phenyl)-2,9-diazaspiro[5.5]undecane-9-carboxylate

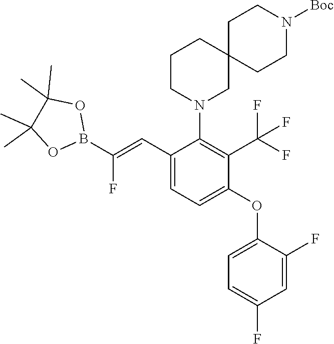 [SMILES: CC(C)(C)OC(=O)N1CCC2(CCCN(c3c(/C=C(\F)B4OC(C)(C)C(C)(C)O4)ccc(Oc4ccc(F)cc4F)c3C(F)(F)F)C2)CC1]

The title compound was prepared according to the procedures described in Intermediate 19, with 2,4-difluorophenol replacing phenol in step 1. LC-MS calculated for C₂₉H₃₄BF₆N₂O₅ (M+H−C₆H₁₀)⁺: m/z=615.2; found 615.2.

### Intermediate 26. tert-Butyl (S,Z)-((1-(6-(2-fluoro-2-(4,4,5,5-tetramethyl-1,3,2-dioxaborolan-2-yl)vinyl)-3-phenoxy-2-(trifluoromethyl)phenyl)piperidin-3-yl)methyl)carbamate

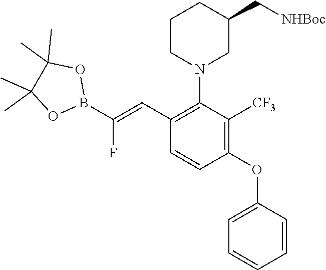 [SMILES: CC(C)(C)OC(=O)NC[C@@H]1CCCN(c2c(/C=C(\F)B3OC(C)(C)C(C)(C)O3)ccc(Oc3ccccc3)c2C(F)(F)F)C1]

The title compound was prepared according to the procedures described in Intermediate 19, with tert-butyl (R)-(piperidin-3-ylmethyl)carbamate replacing tert-butyl 2,9-diazaspiro[5.5]undecane-9-carboxylate in step 1. LC-MS calculated for C₂₆H₃₂BF₄N₂O₅ (M+H−C₆H₁₀)⁺: m/z=539.2; found 539.3.

### Intermediate 27. 4-(6-Chloropyrazin-2-yl)isothiazole

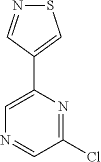 [SMILES: Clc1cncc(-c2cnsc2)n1]

To a mixture of 4-(4,4,5,5-tetramethyl-1,3,2-dioxaborolan-2-yl)isothiazole (1.00 g, 4.74 mmol) in 1,4-dioxane/H₂O (4:1, 15 mL) was added [1,1′ bis(diphenylphosphino)ferrocene] dichloropalladium(II) (0.173 g, 0.237 mmol), 2,6-dichloropyrazine (1.06 g, 7.11 mmol), cesium carbonate (4.63 g, 14.2 mmol)) and the reaction mixture was stirred at 70° C. for 2 hours. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.7 g, 75% yield) as a yellow solid. LC-MS calculated for C₇H5ClN₃S (M+H)⁺: m/z=198.0; found 198.0.

### Intermediate 28. 4-(2-Iodopyrimidin-4-yl)isothiazole

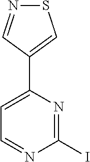 [SMILES: Ic1nccc(-c2cnsc2)n1]

The title compound was prepared according to the procedures described in Intermediate 27, with 2,4-diiodopyrimidine replacing 2,6-dichloropyrazine. LC-MS calculated for C₇H₄IN₃S (M+H)⁺: m/z=289.9; found 289.9.

### Intermediate 29. tert-Butyl (Z)-2-(3-(2,3-difluorophenoxy)-6-(2-fluoro-2-(4,4,5,5-tetramethyl-1,3,2-dioxaborolan-2-yl)vinyl)-2-(trifluoromethyl)phenyl)-2,9-diazaspiro[5.5]undecane-9-carboxylate

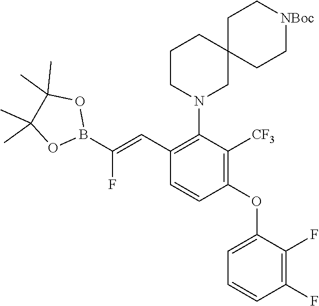 [SMILES: CC(C)(C)OC(=O)N1CCC2(CCCN(c3c(/C=C(\F)B4OC(C)(C)C(C)(C)O4)ccc(Oc4cccc(F)c4F)c3C(F)(F)F)C2)CC1]

The title compound was prepared according to the procedures described in Intermediate 19, with 2,3-difluorophenol replacing phenol. LC-MS calculated for C₂₉H₃₄BF₆N₂O₅ (M+H−C₆H₁₀)⁺: m/z=615.2; found 615.4.

### Intermediate 30. tert-Butyl (Z)-2-(6-(2-fluoro-2-(6-(pyridazin-4-yl)pyrazin-2-yl)vinyl)-3-hydroxy-2-(trifluoromethyl)phenyl)-2,9-diazaspiro[5.5]undecane-9-carboxylate

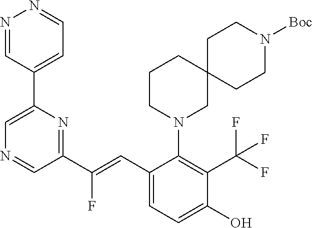 [SMILES: CC(C)(C)OC(=O)N1CCC2(CCCN(c3c(/C=C(\F)c4cncc(-c5ccnnc5)n4)ccc(O)c3C(F)(F)F)C2)CC1]

### Step 1. tert-Butyl 2-(6-nitro-2-(trifluoromethyl)-3-(2-(trimethylsilyl)ethoxy)phenyl)-2,9-diazaspiro[5.5]undecane-9-carboxylate

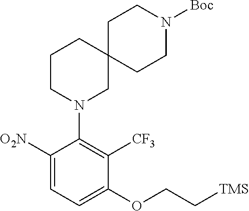 [SMILES: CC(C)(C)OC(=O)N1CCC2(CCCN(c3c([N+](=O)[O-])ccc(OCC[Si](C)(C)C)c3C(F)(F)F)C2)CC1]

To a mixture of 2-(trimethylsilyl)ethan-1-ol (2.59 g, 21.9 mmol) in THF (40 ml) at 0° C. was added sodium hydride (1.31 g, 32.8 mmol, 60% dispersion in mineral oil). The resulting mixture was stirred at 0° C. for 1 hour. After that, 2-bromo-4-fluoro-1-nitro-3-(trifluoromethyl)benzene (6.00 g, 20.8 mmol) was added to the reaction mixture at 0° C. The resulting mixture was stirred at rt for 18 hours. The reaction mixture was poured into a solution of ammonium chloride. The mixture was extracted with EtOAc, dried over sodium sulfate, and concentrated. The crude residue was directly used without further purification. To a mixture of the crude material in acetonitrile (50 mL) was added tert-butyl 2,9-diazaspiro[5.5]undecane-9-carboxylate (7.25 g, 28.5 mmol) and N-ethyl-N-isopropylpropan-2-amine (12.2 mL, 65.7 mmol) and the reaction mixture was stirred at 90° C. for 5 hours. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (5.5 g, 65% yield) as a yellow oil. LC-MS calculated for C₂₆H₄₁F₃N₃O₅Si(M+H)⁺: m/z=560.3; found 560.3.

### Step 2. tert-Butyl 2-(6-amino-2-(trifluoromethyl)-3-(2-(trimethylsilyl)ethoxy)phenyl)-2,9-diazaspiro[5.5]undecane-9-carboxylate

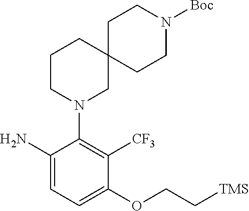 [SMILES: CC(C)(C)OC(=O)N1CCC2(CCCN(c3c(N)ccc(OCC[Si](C)(C)C)c3C(F)(F)F)C2)CC1]

To a mixture of tert-butyl 2-(6-nitro-2-(trifluoromethyl)-3-(2-(trimethylsilyl)ethoxy)phenyl)-2,9-diazaspiro[5.5]undecane-9-carboxylate (7.00 g, 12.5 mmol) in DMF (40 mL) was added hypodiboric acid (3.36 g, 37.5 mmol) and 4,4′-bipyridine (9.8 mg, 0.063 mmol) and the reaction mixture was stirred at rt for 1 hour before the reaction mixture was poured into water. The mixture was extracted with EtOAc, dried over sodium sulfate, and concentrated in vacuo. The crude residue was purified by flash column chromatography (SiO₂, EtOAc/Hexanes) to afford the desired product (4.5 g, 68% yield) as a yellow oil. LC-MS calculated for C₂₆H₄₃F3N₃O₃Si (M+H)⁺: m/z=530.3; found 530.4.

### Step 3. tert-Butyl 2-(6-iodo-2-(trifluoromethyl)-3-(2-(trimethylsilyl)ethoxy)phenyl)-2,9-diazaspiro[5.5]undecane-9-carboxylate

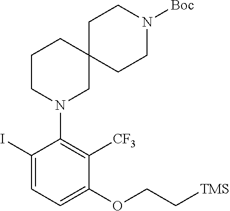 [SMILES: CC(C)(C)OC(=O)N1CCC2(CCCN(c3c(I)ccc(OCC[Si](C)(C)C)c3C(F)(F)F)C2)CC1]

To a mixture of tert-butyl 2-(6-amino-2-(trifluoromethyl)-3-(2-(trimethylsilyl)ethoxy)phenyl)-2,9-diazaspiro[5.5]undecane-9-carboxylate (4.5 g, 8.5 mmol) in ACN/H₂O (5:1, 100 mL) was added acetic acid (7.29 mL, 127 mmol), potassium iodide (7.05 g, 42.5 mmol), and a solution of sodium nitrite (2.93 g, 42.5 mmol) in water (10 mL) and the reaction mixture was stirred at rt for 2 hours. The reaction mixture was poured into a saturated solution of sodium bicarbonate, and sodium thiosulfate. The mixture was extracted with EtOAc, dried over sodium sulfate, and concentrated in vacuo. The crude residue was purified by flash column chromatography (SiO₂, EtOAc/Hexanes) to afford the desired product (3.5 g, 64% yield) as a white waxy solid. LC-MS calculated for C₂₆H₄₁F₃IN₂O₃Si (M+H−C₄H₈)⁺: m/z=641.2; found 641.2.

### Step 4. tert-Butyl 2-(6-(2,2-difluorovinyl)-2-(trifluoromethyl)-3-(2-(trimethylsilyl)ethoxy) phenyl)-2,9-diazaspiro[5.5]undecane-9-carboxylate

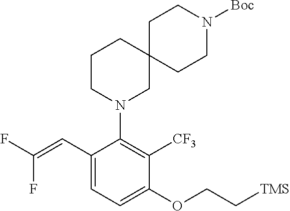 [SMILES: CC(C)(C)OC(=O)N1CCC2(CCCN(c3c(C=C(F)F)ccc(OCC[Si](C)(C)C)c3C(F)(F)F)C2)CC1]

To a mixture of tert-butyl 2-(6-iodo-2-(trifluoromethyl)-3-(2-(trimethylsilyl)ethoxy) phenyl)-2,9-diazaspiro[5.5]undecane-9-carboxylate (2.00 g, 3.12 mmol) in 1,4-dioxane/H₂O (4:1, 20 mL) was added [1,1′-bis(diphenylphosphino)ferrocene]dichloropalladium(II) (0.46 g, 0.62 mmol), 2-(2,2-difluorovinyl)-4,4,5,5-tetramethyl-1,3,2-dioxaborolane (1.78 g, 9.37 mmol), and cesium carbonate (3.05 g, 9.37 mmol) and the reaction mixture was stirred at 70° C. for 4 hours. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (1.0 g, 55% yield) as a white waxy solid. LC-MS calculated for C₂₈H₄₂F₅N₂O₃Si (M+H)⁺: m/z=577.3; found 577.3.

### Step 5. tert-Butyl (Z)-2-(6-(2-fluoro-2-(4,4,5,5-tetramethyl-1,3,2-dioxaborolan-2-yl)vinyl)-2-(trifluoromethyl)-3-(2-(trimethylsilyl)ethoxy)phenyl)-2,9-diazaspiro[5.5]undecane-9-carboxylate

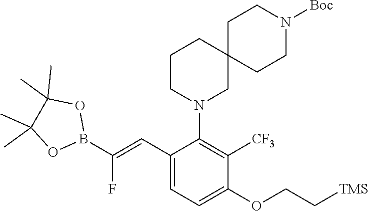 [SMILES: CC(C)(C)OC(=O)N1CCC2(CCCN(c3c(/C=C(\F)B4OC(C)(C)C(C)(C)O4)ccc(OCC[Si](C)(C)C)c3C(F)(F)F)C2)CC1]

To a mixture of tert-butyl 2-(6-(2,2-difluorovinyl)-2-(trifluoromethyl)-3-(2-(trimethylsilyl)ethoxy) phenyl)-2,9-diazaspiro[5.5]undecane-9-carboxylate (1.30 g, 2.25 mmol) in THF (15 mL) was added CuCl(PCy₃)₂(0.15 g, 0.23 mmol), 4,4,4′,4′,5,5,5′,5′-octamethyl-2,2′-bi(1,3,2-dioxaborolane) (1.72 g, 6.76 mmol), and potassium acetate (0.66 g, 6.76 mmol) and the reaction mixture was purged with N₂ and stirred at 70° C. for 2 hours. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (2.1 g, 76% yield) as waxy solid. LC-MS calculated for C₂₈H₄₄BF₄N₂O₅Si (M+H−C₆H₁₀)⁺: m/z=603.3; found 603.4. CuCl(PCy₃)₂ was prepared in accordance with literature procedures (see e.g., Bowmaker et al. J. Chem. Soc., Dalton Trans. 2002, 2722-2730).

### Step 6. tert-Butyl (Z)-2-(6-(2-fluoro-2-(6-(pyridazin-4-yl)pyrazin-2-yl)vinyl)-2-(trifluoromethyl)-3-(2-(trimethylsilyl)ethoxy)phenyl)-2,9-diazaspiro[5.5]undecane-9-carboxylate

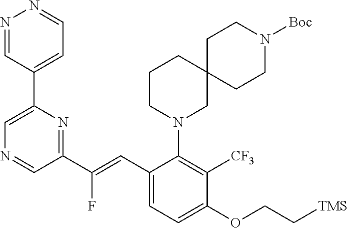 [SMILES: CC(C)(C)OC(=O)N1CCC2(CCCN(c3c(/C=C(\F)c4cncc(-c5ccnnc5)n4)ccc(OCC[Si](C)(C)C)c3C(F)(F)F)C2)CC1]

To a mixture of 4-(6-chloropyrazin-2-yl)pyridazine (0.219 g, 1.14 mmol) (Intermediate 1) in 1,4-dioxane/H₂O (4:1, 10 mL) was added tert-butyl (Z)-2-(6-(2-fluoro-2-(4,4,5,5-tetramethyl-1,3,2-dioxaborolan-2-yl)vinyl)-2-(trifluoromethyl)-3-(2-(trimethylsilyl)ethoxy)phenyl)-2,9-diazaspiro[5.5]undecane-9-carboxylate (0.60 g, 0.88 mmol), cesium carbonate (0.857 g, 2.63 mmol), and chloro(2-dicyclohexylphosphino-2′,4′,6′-triisopropyl-1,1′-biphenyl)[2-(2′-amino-1,1′-biphenyl)]palladium(II) (135 mg, 0.175 mmol) and the reaction mixture was stirred at 100° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.50 g, 80% yield). LC-MS calculated for C₃₆H₄₇F₄N₆O₃Si (M+H)⁺: m/z=715.3; found 715.5.

### Step 7. tert-Butyl (Z)-2-(6-(2-fluoro-2-(6-(pyridazin-4-yl)pyrazin-2-yl)vinyl)-3-hydroxy-2-(trifluoromethyl)phenyl)-2,9-diazaspiro[5.5]undecane-9-carboxylate

To a mixture of tert-butyl (Z)-2-(6-(2-fluoro-2-(6-(pyridazin-4-yl)pyrazin-2-yl)vinyl)-2-(trifluoromethyl)-3-(2-(trimethylsilyl)ethoxy)phenyl)-2,9-diazaspiro[5.5]undecane-9-carboxylate (0.60 g, 0.84 mmol) in DMF (10 mL) was added cesium fluoride (0.255 g, 1.18 mmol) and the reaction mixture was stirred at 80° C. for 2 hours before the reaction mixture was poured into water. The mixture was extracted with EtOAc, dried over sodium sulfate, and concentrated in vacuo. The crude residue was purified by flash column chromatography (SiO₂, EtOAc/Hexanes) to afford the desired product (0.45 g, 87% yield). LC-MS calculated for C₃₁H₃₅F4N₆O₃(M+H)⁺: m/z=615.3; found 615.3.

### Intermediate 31. tert-Butyl (Z)-2-(6-(2-fluoro-2-(4-(pyridazin-4-yl)pyrimidin-2-yl)vinyl)-3-hydroxy-2-(trifluoromethyl)phenyl)-2,9-diazaspiro[5.5]undecane-9-carboxylate

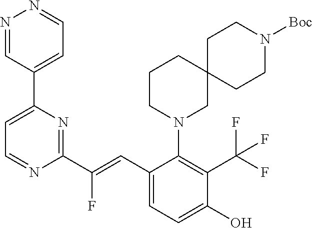 [SMILES: CC(C)(C)OC(=O)N1CCC2(CCCN(c3c(/C=C(\F)c4nccc(-c5ccnnc5)n4)ccc(O)c3C(F)(F)F)C2)CC1]

The title compound was prepared according to the procedures described in Intermediate 30, with 4-(2-iodopyrimidin-4-yl)pyridazine (Intermediate 4) replacing 4-(6-chloropyrazin-2-yl)pyridazine (Intermediate 1) in step 6. LC-MS calculated for C₃₁H₃₅F₄N₆O₃(M+H)⁺: m/z=615.3; found 615.3.

### Example 1. (S,Z)-(1-(6-(2-(5-(3,3-Difluoroazetidin-1-yl)pyrido[3,4-b]pyrazin-7-yl)-2-fluorovinyl)-3-(2-fluorophenoxy)-2-(trifluoromethyl)phenyl)piperidin-3-yl)methanamine

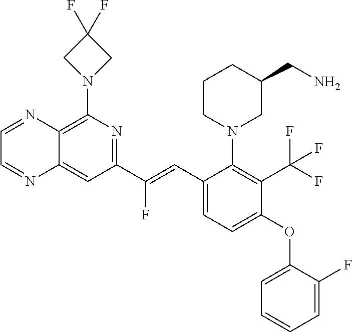 [SMILES: NC[C@@H]1CCCN(c2c(/C=C(\F)c3cc4nccnc4c(N4CC(F)(F)C4)n3)ccc(Oc3ccccc3F)c2C(F)(F)F)C1]

To a mixture of 7-chloro-5-(3,3-difluoroazetidin-1-yl)pyrido[3,4-b]pyrazine (Intermediate 5, 8.04 mg, 0.031 mmol) in 1,4-dioxane/H₂O (5:1) (2 mL) was added chloro(2-dicyclohexylphosphino-2′,4′,6′-triisopropyl-1,1′-biphenyl)[2-(2′-amino-1,1′-biphenyl)]palladium(II) (4.39 mg, 6.26 μmol), tert-butyl (S,Z)-((1-(6-(2-fluoro-2-(4,4,5,5-tetramethyl-1,3,2-dioxaborolan-2-yl)vinyl)-3-(2-fluorophenoxy)-2-(trifluoromethyl)phenyl)piperidin-3-yl)methyl)carbamate (Intermediate 9, 0.02 g, 0.031 mmol), and cesium carbonate (0.031 g, 0.094 mmol) and the reaction mixture was stirred at 110° C. for 30 mins. After cooling to rt, the reaction mixture was diluted with MeOH (1 mL) and filtered over a SiliaPrep SPE silica-based thiol cartridge. A 4 molar solution of HCl in 1,4-dioxane (2 mL, 800 mmol) was added to the filtrate and the reaction mixture was stirred at 40° C. for 30 min. The mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₁H₂₈F₇N₆ (M+H)⁺: m/z=633.2; found 633.4.

### Example 2. (S,Z)-(1-(6-(2-(8-(3,3-Difluoroazetidin-1-yl)imidazo[1,2-a]pyrazin-6-yl)-2-fluorovinyl)-3-(2-fluorophenoxy)-2-(trifluoromethyl)phenyl)piperidin-3-yl)methanamine

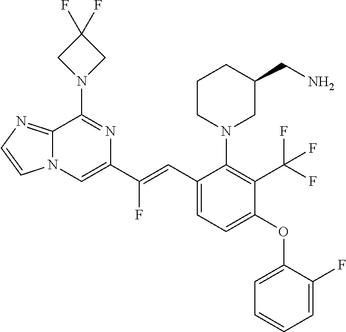 [SMILES: NC[C@@H]1CCCN(c2c(/C=C(\F)c3cn4ccnc4c(N4CC(F)(F)C4)n3)ccc(Oc3ccccc3F)c2C(F)(F)F)C1]

This compound was prepared according to the procedures described in Example 1, with 6-chloro-8-(3,3-difluoroazetidin-1-yl)imidazo[1,2-a]pyrazine (Intermediate 6) replacing 7-chloro-5-(3,3-difluoroazetidin-1-yl)pyrido[3,4-b]pyrazine. LC-MS calculated for C₃₀H₂₈F₇N₆O (M+H)⁺: m/z=621.2; found 621.2.

### Example 3. (S,Z)-3-(2-(2-(3-(Aminomethyl)piperidin-1-yl)-4-(2-fluorophenoxy)-3-(trifluoromethyl)phenyl)-1-fluorovinyl)-5-(3,3-difluoroazetidin-1-yl)pyrazine-2-carbonitrile

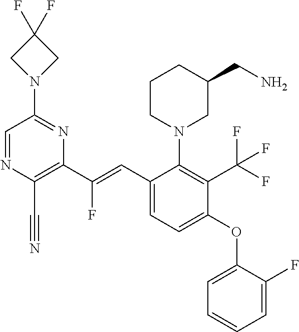 [SMILES: N#Cc1ncc(N2CC(F)(F)C2)nc1/C(F)=C/c1ccc(Oc2ccccc2F)c(C(F)(F)F)c1N1CCC[C@@H](CN)C1]

This compound was prepared according to the procedures described in Example 1, with 3-chloro-5-(3,3-difluoroazetidin-1-yl)pyrazine-2-carbonitrile (Intermediate 7) replacing 7-chloro-5-(3,3-difluoroazetidin-1-yl)pyrido[3,4-b]pyrazine. LC-MS calculated for C₂₉H₂₆F₇N₆O (M+H)⁺: m/z=607.2; found 607.3.

### Example 4. (S,Z)-(1-(6-(2-Fluoro-2-(6-(isothiazol-5-yl)pyrazin-2-yl)vinyl)-3-(2-fluorophenoxy)-2-(trifluoromethyl)phenyl)piperidin-3-yl)methanamine

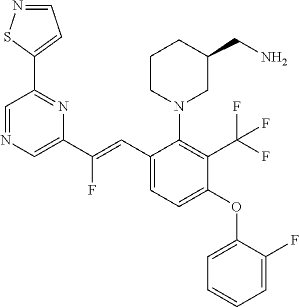 [SMILES: NC[C@@H]1CCCN(c2c(/C=C(\F)c3cncc(-c4ccns4)n3)ccc(Oc3ccccc3F)c2C(F)(F)F)C1]

### Step 1. 2-Ethynyl-6-(methylthio)pyrazine

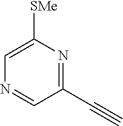

To a mixture of 2-chloro-6-(methylthio)pyrazine (3 g, 18.68 mmol) in Et₃N (40 mL) was added tetrakis(triphenylphosphine)palladium(0) (2.158 g, 1.868 mmol), copper(I) iodide (0.356 g, 1.868 mmol), and ethynyltrimethylsilane (3.88 mL, 28.0 mmol). The reaction mixture was stirred at 40° C. for 5 hours. After cooling to rt, the reaction mixture was filtered through SiO₂, the filter cake was washed with EtOAc, and the filtrate was concentrated in vacuo. To a mixture of the crude residue in THF was added tetra-N-butylammonium fluoride (56.0 mL, 56.0 mmol) and the reaction mixture was stirred at rt for 30 mins. The mixture was concentrated in vacuo and the crude residue was purified by flash column chromatography (SiO₂, EtOAc/Hexanes) to afford the desired product (1.0 g, 36% yield). LC-MS calculated for C₇H7N₂S (M+H)⁺: m/z=151.0; found 150.9.

### Step 2. 2-(Bromoethynyl)-6-(methylthio)pyrazine

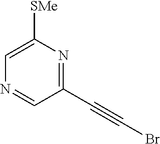 [SMILES: CSc1cncc(C#CBr)n1]

To a mixture of 2-ethynyl-6-(methylthio)pyrazine (0.56 g, 3.73 mmol) in acetonitrile (20 mL) was added NBS (0.796 g, 4.47 mmol), and DBU (0.681 g, 4.47 mmol) and the reaction mixture was stirred at rt for 30 mins. The reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.43 g, 50% yield). LC-MS calculated for C₇H6BrN₂S (M+H)⁺: m/z=228.9; found 229.0.

### Step 3. (Z)-2-(2-Bromo-1-fluorovinyl)-6-(methylthio)pyrazine

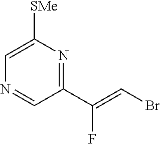 [SMILES: CSc1cncc(/C(F)=C/Br)n1]

To a mixture of 2-(bromoethynyl)-6-(methylthio)pyrazine (0.60 g, 2.6 mmol) in acetonitrile/H₂O (20:1) (10 mL) was added silver (I) fluoride (0.831 g, 6.55 mmol) and the reaction mixture was stirred at 70° C. overnight. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.07 g, 11% yield). LC-MS calculated for C₇H₇BrFN₂S (M+H)⁺: m/z=248.9; found 249.0.

### Step 4. tert-Butyl (S,Z)-((1-(6-(2-fluoro-2-(6-(methylthio)pyrazin-2-yl)vinyl)-3-(2-fluorophenoxy)-2-(trifluoromethyl)phenyl)piperidin-3-yl)methyl)carbamate

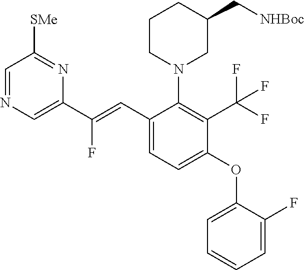 [SMILES: CSc1cncc(/C(F)=C/c2ccc(Oc3ccccc3F)c(C(F)(F)F)c2N2CCC[C@@H](CNC(=O)OC(C)(C)C)C2)n1]

To a mixture of (Z)-2-(2-bromo-1-fluorovinyl)-6-(methylthio)pyrazine (0.07 g, 0.28 mmol) in 1,4-dioxane (4 mL) was added 4,4,4′,4′,5,5,5′,5′-octamethyl-2,2′-bi(1,3,2-dioxaborolane) (0.143 g, 0.562 mmol), potassium acetate (0.110 g, 1.124 mmol), and [1,1′-bis(diphenylphosphino)ferrocene]dichloropalladium(II) (0.041 g, 0.056 mmol) and the reaction mixture was stirred at 90° C. for 2 hours. After cooling to rt, the reaction mixture was filtered through a pad of Celite, and the filter cake was washed with 1,4-dioxane. To the filtrate was added tert-butyl (S)-((1-(3-(2-fluorophenoxy)-6-iodo-2-(trifluoromethyl)phenyl)piperidin-3-yl)methyl)carbamate (0.140 g, 0.236 mmol) (Intermediate 9, Step 3), cesium carbonate (0.231 g, 0.709 mmol), and [1,1′-bis(diphenylphosphino)ferrocene]dichloropalladium(II) (0.035 g, 0.047 mmol) and the reaction mixture was stirred at 130° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.1 g, 66% yield). LC-MS calculated for C₃₁H₃₄F₅N₄O₃S (M+H)⁺: m/z=637.2; found 637.3.

### Step 5. (S,Z)-(1-(6-(2-Fluoro-2-(6-(isothiazol-5-yl)pyrazin-2-yl)vinyl)-3-(2-fluorophenoxy)-2-(trifluoromethyl)phenyl)piperidin-3-yl)methanamine

To a mixture of tert-butyl (S,Z)-((1-(6-(2-fluoro-2-(6-(methylthio)pyrazin-2-yl)vinyl)-3-(2-fluorophenoxy)-2-(trifluoromethyl)phenyl)piperidin-3-yl)methyl)carbamate (0.02 g, 0.031 mmol) in 1,4-dioxane (1 mL) was added isothiazol-5-ylboronic acid (0.012 g, 0.094 mmol), ((2-hydroxy-3-methylbenzoyl)oxy)copper (0.020 g, 0.094 mmol), and chloro[(tricyclohexylphosphine)-2-(2′-aminobiphenyl)]palladium(II) (3.71 mg, 6.28 μmol). The reaction mixture was stirred at 120° C. for 4 hours. After cooling to rt, the reaction mixture was diluted with MeOH (1 mL) and filtered over a SiliaPrep SPE silica-based thiol cartridge. A 4 molar solution of HCl in 1,4-dioxane (2 mL, 800 mmol) was added to the filtrate and the reaction mixture was stirred at 40° C. for 30 min. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt·LC-MS calculated for C₂₈H₂₅F₅N₅OS (M+H)⁺: m/z=574.2; found 574.2.

### Example 5. (Z)-N1-(6-(2-Fluoro-2-(6-(pyridazin-4-yl)pyridin-2-yl)vinyl)-3-phenoxy-2-(trifluoromethyl)phenyl)-N1-methylpropane-1,3-diamine

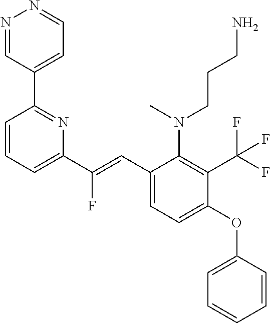 [SMILES: CN(CCCN)c1c(/C=C(\F)c2cccc(-c3ccnnc3)n2)ccc(Oc2ccccc2)c1C(F)(F)F]

The title compound was prepared according to the procedures described in Example 1, with 4-(6-chloropyridin-2-yl)pyridazine (Intermediate 2) replacing 7-chloro-5-(3,3-difluoroazetidin-1-yl)pyrido[3,4-b]pyrazine and tert-butyl (Z)-(3-((6-(2-fluoro-2-(4,4,5,5-tetramethyl-1,3,2-dioxaborolan-2-yl)vinyl)-3-phenoxy-2-(trifluoromethyl)phenyl)(methyl)amino)propyl)carbamate (Intermediate 10) replacing tert-butyl (S,Z)-((1-(6-(2-fluoro-2-(4,4,5,5-tetramethyl-1,3,2-dioxaborolan-2-yl)vinyl)-3-(2-fluorophenoxy)-2-(trifluoromethyl)phenyl)piperidin-3-yl)methyl)carbamate. LC-MS calculated for C₂₈H₂₆F₄N₅O (M+H)⁺: m/z=524.2; found 524.3.

### Example 6. (S,Z)-(1-(2-Chloro-6-(2-fluoro-2-(4-(pyridazin-4-yl)pyrimidin-2-yl)vinyl)-3-(4-fluoro-2-methoxyphenoxy)phenyl)piperidin-3-yl)methanamine

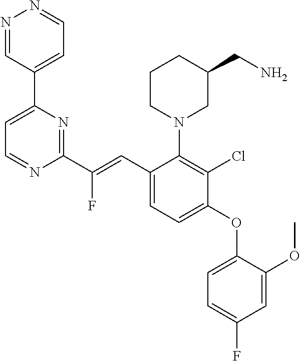 [SMILES: COc1cc(F)ccc1Oc1ccc(/C=C(\F)c2nccc(-c3ccnnc3)n2)c(N2CCC[C@@H](CN)C2)c1Cl]

To a mixture of 4-(2-iodopyrimidin-4-yl)pyridazine (8.95 mg, 0.031 mmol) (Intermediate 4) in 1,4-dioxane/H₂O (5:1) (2 mL) was added [1,1′-bis(diphenylphosphino)ferrocene]dichloropalladium(II) (4.60 mg, 6.30 μmol)), tert-butyl (S,Z)-((1-(2-chloro-6-(2-fluoro-2-(4,4,5,5-tetramethyl-1,3,2-dioxaborolan-2-yl)vinyl)-3-(4-fluoro-2-methoxyphenoxy)phenyl)piperidin-3-yl)methyl)carbamate (0.02 g, 0.031 mmol) (Intermediate 12), and cesium carbonate (0.031 g, 0.094 mmol) and the reaction mixture was stirred at 90° C. for 1 hour. After cooling to rt, the reaction mixture was diluted with MeOH (1 mL) and filtered over a SiliaPrep SPE silica-based thiol cartridge. A 4 molar solution of HCl in 1,4-dioxane (2 mL, 800 mmol) was added to the filtrate and the reaction mixture was stirred at 40° C. for 30 min. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₉H₂₈ClF₂N₆O₂ (M+H)⁺: m/z=565.2; found 565.4.

### Example 7. (S,Z)-(1-(2-Chloro-6-(2-fluoro-2-(2-(pyridazin-4-yl)pyrimidin-4-yl)vinyl)-3-(4-fluoro-2-methoxyphenoxy)phenyl)piperidin-3-yl)methanamine

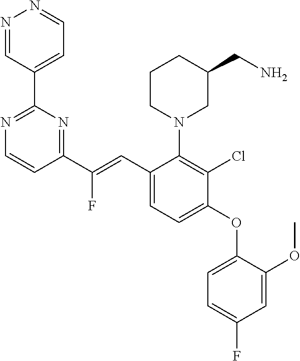 [SMILES: COc1cc(F)ccc1Oc1ccc(/C=C(\F)c2ccnc(-c3ccnnc3)n2)c(N2CCC[C@@H](CN)C2)c1Cl]

This compound was prepared according to the procedures described in Example 6, with 4-(4-chloropyrimidin-2-yl)pyridazine (Intermediate 3) replacing 4-(2-iodopyrimidin-4-yl)pyridazine. LC-MS calculated for C₂₉H₂₈ClF₂N₆O₂ (M+H)⁺: m/z=565.2; found 565.3.

### Example 8. (S,Z)-(1-(2-Chloro-6-(2-(5-(3,3-difluoropyrrolidin-1-yl)-1,2,4-triazin-3-yl)-2-fluorovinyl)-3-(4-fluoro-2-methoxyphenoxy)phenyl)piperidin-3-yl)methanamine

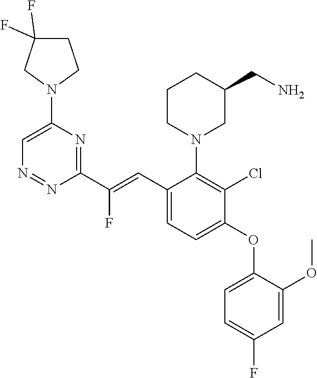 [SMILES: COc1cc(F)ccc1Oc1ccc(/C=C(\F)c2nncc(N3CCC(F)(F)C3)n2)c(N2CCC[C@@H](CN)C2)c1Cl]

This compound was prepared according to the procedures described in Example 6, with 3-chloro-5-(3,3-difluoropyrrolidin-1-yl)-1,2,4-triazine (Intermediate 8) replacing 4-(2-iodopyrimidin-4-yl)pyridazine. LC-MS calculated for C₂₈H₃₀ClF₄N₆O₂ (M+H)⁺: m/z=593.2; found 593.3.

### Example 9. (Z)-4-(6-(2-Fluoro-2-(6-(pyridazin-4-yl)pyrazin-2-yl)vinyl)-3-phenoxy-2-(trifluoromethyl)phenyl)-1-oxa-4,9-diazaspiro[5.5]undecane

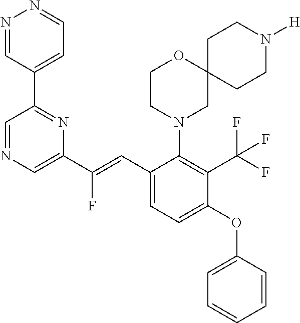 [SMILES: F/C(=C\c1ccc(Oc2ccccc2)c(C(F)(F)F)c1N1CCOC2(CCNCC2)C1)c1cncc(-c2ccnnc2)n1]

Step 1. tert-Butyl (Z)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane-9-carboxylate

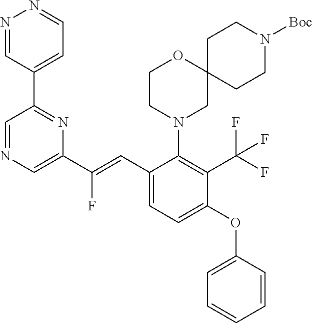 [SMILES: CC(C)(C)OC(=O)N1CCC2(CC1)CN(c1c(/C=C(\F)c3cncc(-c4ccnnc4)n3)ccc(Oc3ccccc3)c1C(F)(F)F)CCO2]

To a mixture of 4-(6-chloropyrazin-2-yl)pyridazine (0.073 g, 0.38 mmol) (Intermediate 1) in 1,4-dioxane (4 mL) was added tert-butyl (Z)-4-(6-(2-fluoro-2-(4,4,5,5-tetramethyl-1,3,2-dioxaborolan-2-yl)vinyl)-3-phenoxy-2-(trifluoromethyl)phenyl)-1-oxa-4,9-diazaspiro[5.5]undecane-9-carboxylate (0.25 g, 0.38 mmol) (Intermediate 11), cesium carbonate (0.369 g, 1.13 mmol), and [1,1′-bis(diphenylphosphino)ferrocene]dichloropalladium(II) (0.041 g, 0.057 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.15 g, 57% yield). LC-MS calculated for C₃₆H₃₇F₄N₆O₄ (M+H)⁺: m/z=693.3; found 693.4.

### Step 2. (Z)-4-(6-(2-Fluoro-2-(6-(pyridazin-4-yl)pyrazin-2-yl)vinyl)-3-phenoxy-2-(trifluoromethyl)phenyl)-1-oxa-4,9-diazaspiro[5.5]undecane

To a solution of tert-butyl (Z)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane-9-carboxylate (0.02 g, 0.029 mmol) in MeOH (1 mL) was added 4 molar solution of HCl in 1,4-dioxane (1 mL, 400 mmol) and the reaction mixture was stirred at 40° C. for 30 min. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₁H₂₉F₄N₆O₂(M+H)⁺: m/z=593.2; found 593.3.

### Example 10. (Z)-9-(Cyclopropylmethyl)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane

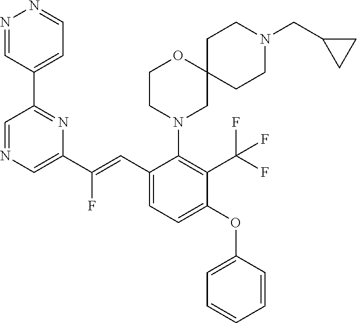 [SMILES: F/C(=C\c1ccc(Oc2ccccc2)c(C(F)(F)F)c1N1CCOC2(CCN(CC3CC3)CC2)C1)c1cncc(-c2ccnnc2)n1]

To a solution of (Z)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane (0.25 g, 0.422 mmol) (Example 9) in acetonitrile (5 mL) was added N-ethyl-N-isopropylpropan-2-amine (0.78 ml, 4.22 mmol) and (iodomethyl)cyclopropane (0.384 g, 2.11 mmol) and the reaction mixture was stirred at 45° C. for 1 hour. After cooling to rt, the reaction mixture was diluted with acetonitrile, water, and several drops of TFA, and the mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₅H₃₅F₄N₆O₂(M+H)⁺: m/z=647.3; found 647.4. ¹H NMR (600 MHz, DMF-d₇) δ 10.16-10.11 (m, 1H), 9.61 (s, 1H), 9.56 (dd, J=5.4, 1.2 Hz, 1H), 9.11 (s, 1H), 8.56 (dd, J=5.4, 2.4 Hz, 1H), 8.13 (d, J=8.8 Hz, 1H), 7.85 (d, J=38.1 Hz, 1H), 7.54-7.48 (m, 2H), 7.27 (t, J=7.4 Hz, 1H), 7.18-7.13 (m, 2H), 7.11 (d, J=8.8 Hz, 1H), 4.15-3.87 (m, 2H), 3.77-3.18 (m, 8H), 3.16 (d, J=7.3 Hz, 2H), 2.61-2.37 (m, 2H), 2.17-1.92 (m, 2H), 1.27-1.16 (m, 1H), 0.70 (d, J=7.8 Hz, 2H), 0.47 (d, J=5.0 Hz, 2H).

### Example 11. (Z)-4-(6-(2-Fluoro-2-(6-(pyridazin-4-yl)pyrazin-2-yl)vinyl)-3-phenoxy-2-(trifluoromethyl)phenyl)-9-(2-methoxyethyl)-1-oxa-4,9-diazaspiro[5.5]undecane

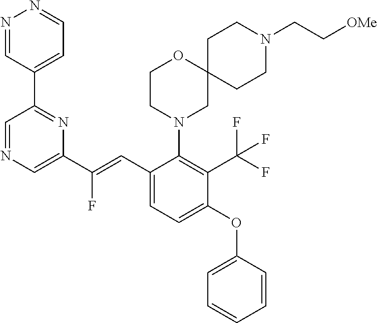 [SMILES: COCCN1CCC2(CC1)CN(c1c(/C=C(\F)c3cncc(-c4ccnnc4)n3)ccc(Oc3ccccc3)c1C(F)(F)F)CCO2]

To a solution of (Z)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane (0.05 g, 0.084 mmol) (Example 9) in acetonitrile (3 mL) was added N-ethyl-N-isopropylpropan-2-amine (0.156 ml, 0.844 mmol) and 1-iodo-2-methoxyethane (0.047 g, 0.253 mmol) and the reaction mixture was stirred at 60° C. for 1 hour. After cooling to rt, the mixture was diluted with MeOH, filtered, and purified by prep-HPLC (Sunfire C18 column, eluting with a gradient of acetonitrile/water containing 0.1% NH₄OH, at flow rate of 60 mL/min) to afford the desired product. LC-MS calculated for C₃₄H₃₅F₄N₆O₃(M+H)⁺: m/z=651.3; found 651.4. ¹H NMR (500 MHz, DMSO-d₆) δ 10.00 (dd, J=2.4, 1.2 Hz, 1H), 9.54 (s, 1H), 9.49 (dd, J=5.4, 1.2 Hz, 1H), 9.08 (s, 1H), 8.41 (dd, J=5.4, 2.4 Hz, 1H), 8.02 (d, J=8.8 Hz, 1H), 7.76 (d, J=38.9 Hz, 1H), 7.50-7.42 (m, 2H), 7.27-7.19 (m, 1H), 7.13-7.07 (m, 2H), 6.95 (d, J=8.8 Hz, 1H), 4.43-3.84 (m, 1H), 3.79-3.39 (m, 1H), 3.37 (t, J=5.9 Hz, 2H), 3.21 (s, 3H), 3.17-2.98 (m, 4H), 2.48-2.13 (m, 7H), 1.80-1.65 (m, 1H), 1.63-1.49 (m, 2H).

### Example 12. (R,Z)-(1-(2-Chloro-6-(2-fluoro-2-(6-(pyridazin-4-yl)pyrazin-2-yl)vinyl)-3-phenoxyphenyl)pyrrolidin-2-yl)methanamine

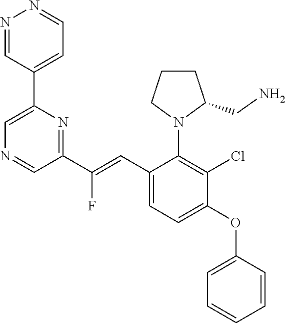 [SMILES: NC[C@H]1CCCN1c1c(/C=C(\F)c2cncc(-c3ccnnc3)n2)ccc(Oc2ccccc2)c1Cl]

### Step 1. tert-Butyl (R,Z)-((1-(2-chloro-6-(2-fluoro-2-(6-(pyridazin-4-yl)pyrazin-2-yl)vinyl)-3-phenoxyphenyl)pyrrolidin-2-yl)methyl)carbamate

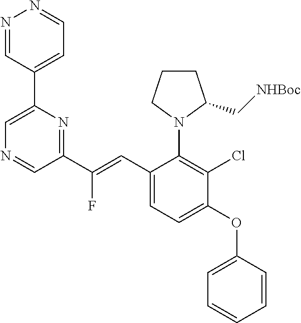 [SMILES: CC(C)(C)OC(=O)NC[C@H]1CCCN1c1c(/C=C(\F)c2cncc(-c3ccnnc3)n2)ccc(Oc2ccccc2)c1Cl]

To a mixture of 4-(6-chloropyrazin-2-yl)pyridazine (0.050 g, 0.262 mmol) (Intermediate 1) in 1,4-dioxane/H₂O (5:1) (10 mL) was added [1,1′-bis(diphenylphosphino)ferrocene]dichloropalladium(II) (0.019 g, 0.026 mmol), tert-butyl (R,Z)-((1-(2-chloro-6-(2-fluoro-2-(4,4,5,5-tetramethyl-1,3,2-dioxaborolan-2-yl)vinyl)-3-phenoxyphenyl)pyrrolidin-2-yl)methyl)carbamate (0.15 g, 0.262 mmol) (Intermediate 13), and cesium carbonate (0.256 g, 0.785 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.1 g, 63% yield). LC-MS calculated for C₃₂H₃₃ClFN₆O₃(M+H)⁺: m/z=603.2; found 603.2.

### Step 2: (R,Z)-(1-(2-Chloro-6-(2-fluoro-2-(6-(pyridazin-4-yl)pyrazin-2-yl)vinyl)-3-phenoxyphenyl)pyrrolidin-2-yl)methanamine

To a mixture of tert-butyl (R,Z)-((1-(2-chloro-6-(2-fluoro-2-(6-(pyridazin-4-yl)pyrazin-2-yl)vinyl)-3-phenoxyphenyl)pyrrolidin-2-yl)methyl)carbamate in MeOH (1 mL) was added a 4 molar solution of HCl in 1,4-dioxane (2 mL, 800 mmol) and the reaction mixture was stirred at 40° C. for 30 min. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₇H₂₅ClFN₆O (M+H)⁺: m/z=503.2; found 503.2. ¹H NMR (600 MHz, DMSO-d₆) δ 10.00 (dd, J=2.4, 1.2 Hz, 1H), 9.56 (s, 1H), 9.53 (dd, J=5.4, 1.2 Hz, 1H), 9.12 (s, 1H), 8.43 (dd, J=5.4, 2.4 Hz, 1H), 7.97 (d, J=8.8 Hz, 1H), 7.66 (s, 4H), 7.51-7.43 (m, 2H), 7.27-7.21 (m, 1H), 7.11-7.04 (m, 3H), 3.99-3.89 (m, 1H), 3.51-3.17 (m, 2H), 2.79-2.65 (m, 2H), 2.35-2.24 (m, 1H), 2.19-1.98 (m, 3H).

### Example 13. (R,Z)-(1-(2-Chloro-6-(2-fluoro-2-(4-(pyridazin-4-yl)pyrimidin-2-yl)vinyl)-3-phenoxyphenyl)pyrrolidin-2-yl)methanamine

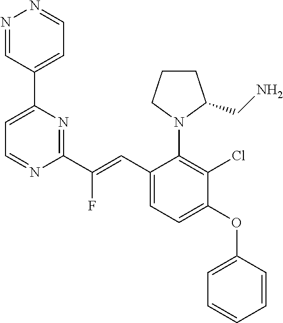 [SMILES: NC[C@H]1CCCN1c1c(/C=C(\F)c2nccc(-c3ccnnc3)n2)ccc(Oc2ccccc2)c1Cl]

This compound was prepared according to the procedures described in Example 12, with 4-(2-iodopyrimidin-4-yl)pyridazine (Intermediate 4) replacing 4-(6-chloropyrazin-2-yl)pyridazine. LC-MS calculated for C₂₇H₂₅ClFN₆O (M+H)⁺: m/z=503.2; found 503.2. ¹H NMR (500 MHz, DMSO-d₆) δ 10.01 (dd, J=2.4, 1.3 Hz, 1H), 9.55 (dd, J=5.4, 1.3 Hz, 1H), 9.17 (d, J=5.2 Hz, 1H), 8.44 (dd, J=5.4, 2.4 Hz, 1H), 8.35 (d, J=5.2 Hz, 1H), 7.99 (d, J=8.8 Hz, 1H), 7.74-7.58 (m, 4H), 7.51-7.41 (m, 2H), 7.28-7.20 (m, 1H), 7.12-7.03 (m, 3H), 4.00-3.87 (m, 1H), 3.31-3.23 (m, 1H), 2.78-2.62 (m, 2H), 2.36-2.24 (m, 1H), 2.20-2.00 (m, 4H).

### Example 14. (Z)-4-(3-Cyclopropoxy-6-(2-fluoro-2-(6-(pyridazin-4-yl)pyrazin-2-yl)vinyl)-2-(trifluoromethyl)phenyl)-9-ethyl-1-oxa-4,9-diazaspiro[5.5]undecane

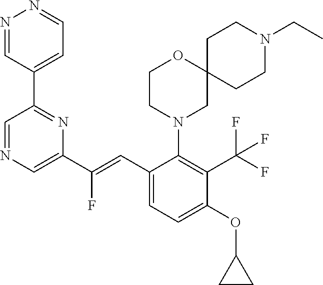 [SMILES: CCN1CCC2(CC1)CN(c1c(/C=C(\F)c3cncc(-c4ccnnc4)n3)ccc(OC3CC3)c1C(F)(F)F)CCO2]

### Step 1. (Z)-4-(3-Cyclopropoxy-6-(2-fluoro-2-(6-(pyridazin-4-yl)pyrazin-2-yl)vinyl)-2-(trifluoromethyl)phenyl)-1-oxa-4,9-diazaspiro[5.5]undecane

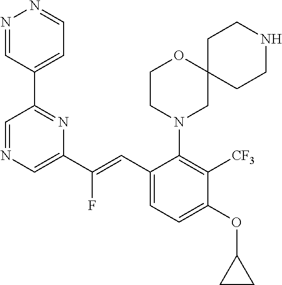 [SMILES: F/C(=C\c1ccc(OC2CC2)c(C(F)(F)F)c1N1CCOC2(CCNCC2)C1)c1cncc(-c2ccnnc2)n1]

This compound was prepared according to the procedures described in Example 9, with tert-butyl (Z)-4-(3-cyclopropoxy-6-(2-fluoro-2-(4,4,5,5-tetramethyl-1,3,2-dioxaborolan-2-yl)vinyl)-2-(trifluoromethyl)phenyl)-1-oxa-4,9-diazaspiro[5.5]undecane-9-carboxylate (Intermediate 15) replacing tert-butyl (Z)-4-(6-(2-fluoro-2-(4,4,5,5-tetramethyl-1,3,2-dioxaborolan-2-yl)vinyl)-3-phenoxy-2-(trifluoromethyl)phenyl)-1-oxa-4,9-diazaspiro[5.5]undecane-9-carboxylate. LC-MS calculated for C₂₈H₂₉F₄N₆O₂ (M+H)⁺: m/z=557.2; found 557.2.

### Step 2. (Z)-4-(3-Cyclopropoxy-6-(2-fluoro-2-(6-(pyridazin-4-yl)pyrazin-2-yl)vinyl)-2-(trifluoromethyl)phenyl)-9-ethyl-1-oxa-4,9-diazaspiro[5.5]undecane

To a mixture of (Z)-4-(3-cyclopropoxy-6-(2-fluoro-2-(6-(pyridazin-4-yl)pyrazin-2-yl)vinyl)-2-(trifluoromethyl)phenyl)-1-oxa-4,9-diazaspiro[5.5]undecane (0.020 g, 0.036 mmol) and N-ethyl-N-isopropylpropan-2-amine (0.031 mL, 0.18 mmol) in acetonitrile (0.5 mL) was added iodoethane (0.056 g, 0.36 mmol) dropwise, and the mixture was stirred at 40° C. for 30 mins. After cooling to rt, the reaction mixture was diluted with acetonitrile, water, and several drops of TFA, and the mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₀H₃₃F₄N₆O₂(M+H)⁺: m/z=585.2; found 585.2.

### Example 15. (Z)-9-(Cyclopropylmethyl)-2-(6-(2-(6-(3,3-difluoroazetidin-1-yl)pyrazin-2-yl)-2-fluorovinyl)-3-phenoxy-2-(trifluoromethyl)phenyl)-2,9-diazaspiro[5.5]undecane

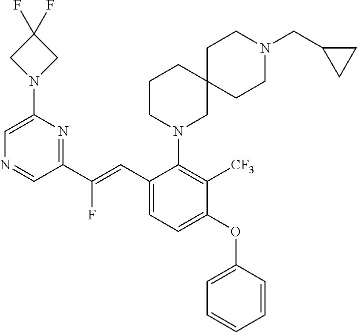 [SMILES: F/C(=C\c1ccc(Oc2ccccc2)c(C(F)(F)F)c1N1CCCC2(CCN(CC3CC3)CC2)C1)c1cncc(N2CC(F)(F)C2)n1]

### Step 1. tert-Butyl (Z)-2-(6-(2-(6-(3,3-difluoroazetidin-1-yl)pyrazin-2-yl)-2-fluorovinyl)-3-phenoxy-2-(trifluoromethyl)phenyl)-2,9-diazaspiro[5.5]undecane-9-carboxylate

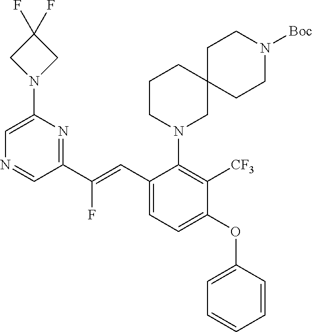 [SMILES: CC(C)(C)OC(=O)N1CCC2(CCCN(c3c(/C=C(\F)c4cncc(N5CC(F)(F)C5)n4)ccc(Oc4ccccc4)c3C(F)(F)F)C2)CC1]

To a mixture of 2-bromo-6-(3,3-difluoroazetidin-1-yl)pyrazine (0.18 g, 0.73 mmol) (Intermediate 17) in 1,4-dioxane/H₂O (4:1)(10 mL) was added tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (0.40 g, 0.61 mmol) (Intermediate 19), cesium carbonate (0.59 g, 1.8 mmol), and chloro(2-dicyclohexylphosphino-2′,4′,6′-triisopropyl-1,1′-biphenyl)[2-(2′-amino-1,1′-biphenyl)]palladium(II) (64 mg, 0.091 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.30 g, 70% yield). LC-MS calculated for C₃₆H₄₀F₆N₅O₃ (M+H)⁺: m/z=704.3; found 704.4.

### Step 2. (Z)-9-(Cyclopropylmethyl)-2-(6-(2-(6-(3,3-difluoroazetidin-1-yl)pyrazin-2-yl)-2-fluorovinyl)-3-phenoxy-2-(trifluoromethyl)phenyl)-2,9-diazaspiro[5.5]undecane

To a solution of tert-butyl (Z)-2-(6-(2-(6-(3,3-difluoroazetidin-1-yl)pyrazin-2-yl)-2-fluorovinyl)-3-phenoxy-2-(trifluoromethyl)phenyl)-2,9-diazaspiro[5.5]undecane-9-carboxylate (0.20 g, 0.33 mmol) in DCM (2 mL) was added TFA/DCM 1:1 (2 mL) and the reaction mixture was stirred at 40° C. for 30 min. After cooling to rt, the solvent was concentrated under reduced pressure, and to the crude residue was added acetonitrile (5 mL), N-ethyl-N-isopropylpropan-2-amine (0.61 mL, 3.3 mmol) and (iodomethyl)cyclopropane (300 mg, 1.66 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₅H₃₈F₆N₅O (M+H)⁺: m/z=658.3; found 658.3. ¹H NMR (500 MHz, DMSO-d₆) δ 9.29-9.00 (m, 1H), 8.30-8.26 (m, 1H), 8.19-8.14 (m, 1H), 8.02-7.95 (m, 1H), 7.51-7.40 (m, 2H), 7.40-7.26 (m, 1H), 7.26-7.19 (m, 1H), 7.13-7.02 (m, 2H), 6.94-6.86 (m, 1H), 4.71-4.48 (m, 4H), 3.50-3.26 (m, 2H), 3.22-2.76 (m, 7H), 2.20-1.73 (m, 4H), 1.71-1.33 (m, 5H), 1.11-0.92 (m, 1H), 0.71-0.54 (m, 2H), 0.41-0.24 (m, 2H).

### Example 16. (Z)-2-(6-(2-(6-(3,3-Difluoroazetidin-1-yl)pyrazin-2-yl)-2-fluorovinyl)-3-phenoxy-2-(trifluoromethyl)phenyl)-9-ethyl-2,9-diazaspiro[5.5]undecane

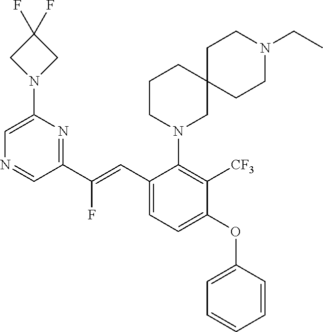 [SMILES: CCN1CCC2(CCCN(c3c(/C=C(\F)c4cncc(N5CC(F)(F)C5)n4)ccc(Oc4ccccc4)c3C(F)(F)F)C2)CC1]

The title compound was prepared according to the procedures described in Example 15, with iodoethane replacing (iodomethyl)cyclopropane. LC-MS calculated for C₃₃H₃₆F₆N₅O (M+H)⁺: m/z=632.3; found 632.4. ¹H NMR (500 MHz, DMSO-d₆) δ 8.26 (s, 1H), 8.14 (s, 1H), 7.95 (d, J=8.8 Hz, 1H), 7.46-7.42 (m, 2H), 7.43-7.35 (m, 1H), 7.24-7.18 (m, 1H), 7.09 (d, J=8.0 Hz, 2H), 6.82 (d, J=8.8 Hz, 1H), 4.67-4.41 (m, 4H), 2.93 (q, J=11.4 Hz, 3H), 2.44-1.99 (m, 7H), 1.81-1.20 (m, 8H), 0.94 (t, J=7.1 Hz, 3H).

### Example 17. (Z)-9-(Cyclopropylmethyl)-2-(6-(2-(6-(3,3-difluoroazetidin-1-yl)pyrazin-2-yl)-2-fluorovinyl)-3-(phenoxy-d5)-2-(trifluoromethyl)phenyl)-2,9-diazaspiro[5.5]undecane

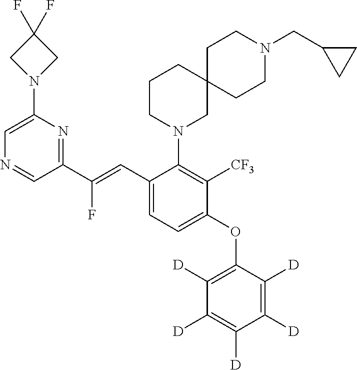 [SMILES: [2H]c1c([2H])c([2H])c(Oc2ccc(/C=C(\F)c3cncc(N4CC(F)(F)C4)n3)c(N3CCCC4(CCN(CC5CC5)CC4)C3)c2C(F)(F)F)c([2H])c1[2H]]

The title compound was prepared according to the procedures described in Example 15, with tert-butyl (Z)-2-(6-(2-fluoro-2-(4,4,5,5-tetramethyl-1,3,2-dioxaborolan-2-yl)vinyl)-3-(phenoxy-d₅)-2-(trifluoromethyl)phenyl)-2,9-diazaspiro[5.5]undecane-9-carboxylate (Intermediate 20) replacing tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (Intermediate 19). LC-MS calculated for C₃₅H₃₃D₅F₆N₅O (M+H)⁺: m/z=663.3; found 663.4.

### Example 18. (Z)-2-(6-(2-(6-(3,3-Difluoroazetidin-1-yl)pyrazin-2-yl)-2-fluorovinyl)-3-(phenoxy-d5)-2-(trifluoromethyl)phenyl)-9-ethyl-2,9-diazaspiro[5.5]undecane

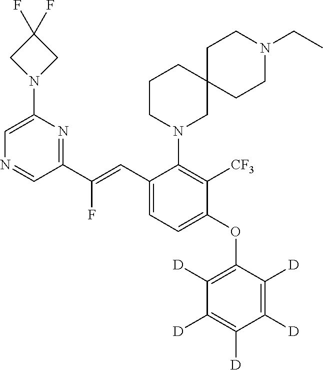 [SMILES: [2H]c1c([2H])c([2H])c(Oc2ccc(/C=C(\F)c3cncc(N4CC(F)(F)C4)n3)c(N3CCCC4(CCN(CC)CC4)C3)c2C(F)(F)F)c([2H])c1[2H]]

The title compound was prepared according to the procedures described in Example 15, with tert-butyl (Z)-2-(6-(2-fluoro-2-(4,4,5,5-tetramethyl-1,3,2-dioxaborolan-2-yl)vinyl)-3-(phenoxy-d₅)-2-(trifluoromethyl)phenyl)-2,9-diazaspiro[5.5]undecane-9-carboxylate (Intermediate 20) replacing tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (Intermediate 19) in step 1 and iodoethane replacing (iodomethyl)cyclopropane in step 2. LC-MS calculated for C₃₃H₃₁D₅F₆N₅O (M+H)⁺: m/z=637.3; found 637.4. ¹H NMR (500 MHz, DMSO-d₆) δ 8.26 (s, 1H), 8.14 (s, 1H), 7.95 (d, J=8.8 Hz, 1H), 7.41 (s, 1H), 6.82 (d, J=8.8 Hz, 1H), 4.66-4.49 (m, 4H), 3.16-2.86 (m, 3H), 2.43-1.99 (m, 7H), 1.77-1.24 (m, 8H), 0.94 (t, J=7.1 Hz, 3H).

### Example 19. (Z)-9-(Cyclopropylmethyl)-2-(6-(2-(6-(3,3-difluoropyrrolidin-1-yl)pyrazin-2-yl)-2-fluorovinyl)-3-phenoxy-2-(trifluoromethyl)phenyl)-2,9-diazaspiro[5.5]undecane

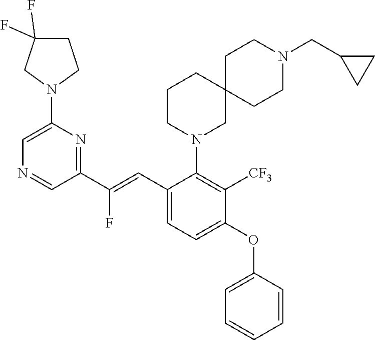 [SMILES: F/C(=C\c1ccc(Oc2ccccc2)c(C(F)(F)F)c1N1CCCC2(CCN(CC3CC3)CC2)C1)c1cncc(N2CCC(F)(F)C2)n1]

### Step 1. tert-Butyl (Z)-2-(6-(2-(6-(3,3-difluoropyrrolidin-1-yl)pyrazin-2-yl)-2-fluorovinyl)-3-phenoxy-2-(trifluoromethyl)phenyl)-2,9-diazaspiro[5.5]undecane-9-carboxylate

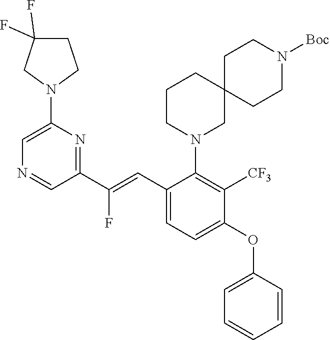 [SMILES: CC(C)(C)OC(=O)N1CCC2(CCCN(c3c(/C=C(\F)c4cncc(N5CCC(F)(F)C5)n4)ccc(Oc4ccccc4)c3C(F)(F)F)C2)CC1]

To a mixture of 2-bromo-6-(3,3-difluoropyrrolidin-1-yl)pyrazine (48 mg, 0.18 mmol) (Intermediate 18) in 1,4-dioxane/H₂O (4:1)(5 mL) was added tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (100 mg, 0.15 mmol) (Intermediate 19), cesium carbonate (148 mg, 0.45 mmol), and chloro(2-dicyclohexylphosphino-2′,4′,6′-triisopropyl-1,1′-biphenyl)[2-(2′-amino-1,1′-biphenyl)]palladium(II) (16 mg, 0.023 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (70 mg, 64% yield). LC-MS calculated for C₃₇H₄₂F₆N₅O₃(M+H)⁺: m/z=718.3; found 718.4.

### Step 2. (Z)-9-(Cyclopropylmethyl)-2-(6-(2-(6-(3,3-difluoropyrrolidin-1-yl)pyrazin-2-yl)-2-fluorovinyl)-3-phenoxy-2-(trifluoromethyl)phenyl)-2,9-diazaspiro[5.5]undecane

To a solution of (Z)-2-(6-(2-(6-(3,3-difluoropyrrolidin-1-yl)pyrazin-2-yl)-2-fluorovinyl)-3-phenoxy-2-(trifluoromethyl)phenyl)-2,9-diazaspiro[5.5]undecane (0.05 g, 0.07 mmol) in DCM (2 mL) was added TFA/DCM 1:1 (2 mL) and the reaction mixture was stirred at 40° C. for 30 min. After cooling to rt, the solvent was concentrated under reduced pressure, and to the crude residue was added acetonitrile (5 mL), N-ethyl-N-isopropylpropan-2-amine (0.15 mL, 0.81 mmol)) and (iodomethyl)cyclopropane (0.074 g, 0.40 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₆H₄₀F₆N₅O (M+H)⁺: m/z=672.3; found 672.4.

### Example 20. (Z)-2-(6-(2-(6-(3,3-Difluoropyrrolidin-1-yl)pyrazin-2-yl)-2-fluorovinyl)-3-phenoxy-2-(trifluoromethyl)phenyl)-9-ethyl-2,9-diazaspiro[5.5]undecane

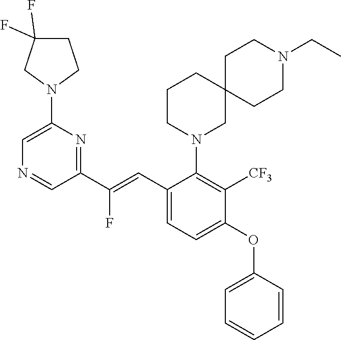 [SMILES: CCN1CCC2(CCCN(c3c(/C=C(\F)c4cncc(N5CCC(F)(F)C5)n4)ccc(Oc4ccccc4)c3C(F)(F)F)C2)CC1]

The title compound was prepared according to the procedures described in Example 19, with iodoethane replacing (iodomethyl)cyclopropane in step 2. LC-MS calculated for C₃₄H₃₈F₆N₅O (M+H)⁺: m/z=646.3; found 646.4. ¹H NMR (500 MHz, DMSO-d₆) δ 8.20-8.15 (m, 2H), 7.93 (d, J=8.8 Hz, 1H), 7.49-7.39 (m, 2H), 7.47-7.35 (m, 1H), 7.21 (t, J=7.4 Hz, 1H), 7.09 (d, J=8.0 Hz, 2H), 6.81 (d, J=8.8 Hz, 1H), 4.03-3.89 (m, 2H), 3.84-3.71 (m, 2H), 3.17-3.08 (m, 1H), 2.99-2.85 (m, 3H), 2.70-2.58 (m, 2H), 2.36-2.16 (m, 6H), 2.10-1.94 (m, 1H), 1.81-1.70 (m, 2H), 1.66-1.48 (m, 2H), 1.47-1.33 (m, 2H), 1.31-1.20 (m, 1H), 0.95 (t, J=7.2 Hz, 3H).

### Example 21. (Z)-9-(Cyclopropylmethyl)-2-(6-(2-(6-(3,3-difluoropyrrolidin-1-yl)pyrazin-2-yl)-2-fluorovinyl)-3-(phenoxy-ds)-2-(trifluoromethyl)phenyl)-2,9-diazaspiro[5.5]undecane

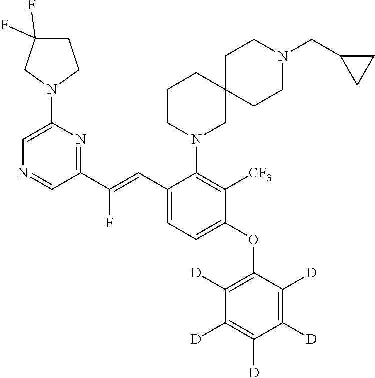 [SMILES: [2H]c1c([2H])c([2H])c(Oc2ccc(/C=C(\F)c3cncc(N4CCC(F)(F)C4)n3)c(N3CCCC4(CCN(CC5CC5)CC4)C3)c2C(F)(F)F)c([2H])c1[2H]]

The title compound was prepared according to the procedures described in Example 19, with tert-butyl (Z)-2-(6-(2-fluoro-2-(4,4,5,5-tetramethyl-1,3,2-dioxaborolan-2-yl)vinyl)-3-(phenoxy-d₅)-2-(trifluoromethyl)phenyl)-2,9-diazaspiro[5.5]undecane-9-carboxylate (Intermediate 20) replacing tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (Intermediate 19). LC-MS calculated for C₃₆H₃₅D₅F₆N₅O (M+H)⁺: m/z=677.3; found 677.4.

### Example 22. (Z)-2-(6-(2-(6-(3,3-Difluoropyrrolidin-1-yl)pyrazin-2-yl)-2-fluorovinyl)-3-(phenoxy-d5)-2-(trifluoromethyl)phenyl)-9-ethyl-2,9-diazaspiro[5.5]undecane

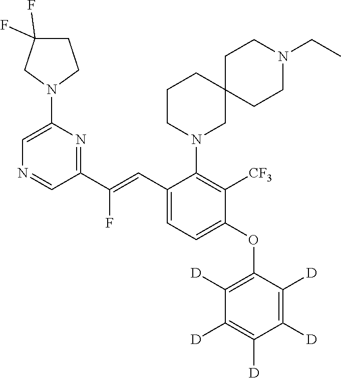 [SMILES: [2H]c1c([2H])c([2H])c(Oc2ccc(/C=C(\F)c3cncc(N4CCC(F)(F)C4)n3)c(N3CCCC4(CCN(CC)CC4)C3)c2C(F)(F)F)c([2H])c1[2H]]

The title compound was prepared according to the procedures described in Example 19, with tert-butyl (Z)-2-(6-(2-fluoro-2-(4,4,5,5-tetramethyl-1,3,2-dioxaborolan-2-yl)vinyl)-3-(phenoxy-d₅)-2-(trifluoromethyl)phenyl)-2,9-diazaspiro[5.5]undecane-9-carboxylate (Intermediate 20) replacing tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (Intermediate 19) in step 1, and iodoethane replacing (iodomethyl)cyclopropane in step 2. LC-MS calculated for C₃₄H₃₃D₅F₆N₅O (M+H)⁺: m/z=651.3; found 651.4. ¹H NMR (500 MHz, DMSO-d₆) δ 8.21-8.14 (m, 2H), 7.93 (d, J=8.8 Hz, 1H), 7.47-7.33 (m, 1H), 6.82 (d, J=8.8 Hz, 1H), 4.04-3.89 (m, 2H), 3.86-3.71 (m, 2H), 3.18-3.08 (m, 1H), 3.00-2.85 (m, 3H), 2.70-2.58 (m, 2H), 2.35-2.18 (m, 6H), 2.08-1.95 (m, 1H), 1.82-1.69 (m, 2H), 1.67-1.48 (m, 2H), 1.47-1.33 (m, 2H), 1.32-1.21 (m, 1H), 0.95 (t, J=7.1 Hz, 3H).

### Example 23. (Z)-9-(Cyclopropylmethyl)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane

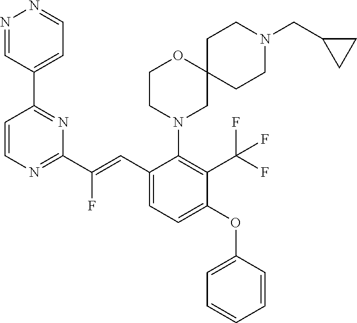 [SMILES: F/C(=C\c1ccc(Oc2ccccc2)c(C(F)(F)F)c1N1CCOC2(CCN(CC3CC3)CC2)C1)c1nccc(-c2ccnnc2)n1]

### Step 1. tert-Butyl (Z)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane-9-carboxylate

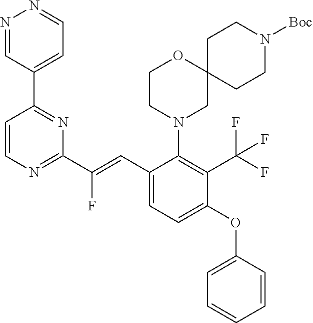 [SMILES: CC(C)(C)OC(=O)N1CCC2(CC1)CN(c1c(/C=C(\F)c3nccc(-c4ccnnc4)n3)ccc(Oc3ccccc3)c1C(F)(F)F)CCO2]

To a mixture of 4-(2-bromopyrimidin-4-yl)pyridazine (0.17 g, 0.72 mmol) (Intermediate 16) in 1,4-dioxane (4 mL) was added tert-butyl (Z)-4-(6-(2-fluoro-2-(4,4,5,5-tetramethyl-1,3,2-dioxaborolan-2-yl)vinyl)-3-phenoxy-2-(trifluoromethyl)phenyl)-1-oxa-4,9-diazaspiro[5.5]undecane-9-carboxylate (0.4 g, 0.6 mmol) (Intermediate 11), cesium carbonate (0.59 g, 1.8 mmol), and [1,1′-bis(diphenylphosphino)ferrocene]dichloropalladium(II) (66 mg, 0.091 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.35 g, 84% yield). LC-MS calculated for C₃₆H₃₇F₄N₆O₄ (M+H)⁺: m/z=693.3; found 693.3.

### Step 2. (Z)-9-(Cyclopropylmethyl)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane

To a solution of tert-butyl (Z)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane-9-carboxylate (0.20 g, 0.29 mmol) in DCM (2 mL) was added TFA/DCM 1:1 (2 mL) and the reaction mixture was stirred at 40° C. for 30 min. After cooling to rt, the solvent was concentrated under reduced pressure, and to the crude residue was added acetonitrile (5 mL), N-ethyl-N-isopropylpropan-2-amine (0.62 mL, 3.4 mmol) and (iodomethyl)cyclopropane (0.31 g, 1.7 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₅H₃₅F₄N₆O₂(M+H)⁺: m/z=647.3; found 647.3. ¹H NMR (600 MHz, DMSO-d₆) δ 10.13-9.97 (m, 1H), 9.59-9.49 (m, 1H), 9.49-9.28 (m, 1H), 9.26-9.13 (m, 1H), 8.56-8.42 (m, 1H), 8.42-8.30 (m, 1H), 8.12-8.04 (m, 1H), 8.00-7.87 (m, 1H), 7.53-7.42 (m, 2H), 7.31-7.21 (m, 1H), 7.12-7.05 (m, 2H), 7.04-6.96 (m, 1H), 4.03-3.73 (m, 2H), 3.51-3.15 (m, 4H), 3.15-2.98 (m, 6H), 2.44-2.17 (m, 2H), 2.02-1.64 (m, 2H), 1.11-0.95 (m, 1H), 0.72-0.52 (m, 2H), 0.43-0.24 (m, 2H).

### Example 24. (Z)-9-Ethyl-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane

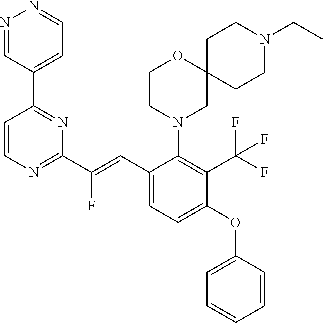 [SMILES: CCN1CCC2(CC1)CN(c1c(/C=C(\F)c3nccc(-c4ccnnc4)n3)ccc(Oc3ccccc3)c1C(F)(F)F)CCO2]

The title compound was prepared according to the procedures described in Example 23, with iodoethane replacing (iodomethyl)cyclopropane in step 2. LC-MS calculated for C₃₃H₃₃F₄N₆O₂(M+H)⁺: m/z=621.3; found 621.2. ¹H NMR (600 MHz, DMSO-d₆) δ 10.01-9.99 (m, 1H), 9.53-9.50 (m, 1H), 9.17 (d, J=5.2 Hz, 1H), 8.45-8.42 (m, 1H), 8.34 (d, J=5.2 Hz, 1H), 8.05 (d, J=8.8 Hz, 1H), 8.01-7.92 (m, 1H), 7.52-7.43 (m, 2H), 7.26-7.19 (m, 1H), 7.17-7.07 (m, 2H), 6.95 (d, J=8.8 Hz, 1H), 4.08-3.86 (m, 1H), 3.86-3.61 (m, 1H), 3.25-2.96 (m, 4H), 2.49-2.09 (m, 7H), 1.80-1.49 (m, 3H), 0.95 (t, J=7.1 Hz, 3H).

### Example 25. (Z)-4-(6-(2-Fluoro-2-(4-(pyridazin-4-yl)pyrimidin-2-yl)vinyl)-3-phenoxy-2-(trifluoromethyl)phenyl)-9-(2-methoxyethyl)-1-oxa-4,9-diazaspiro[5.5]undecane

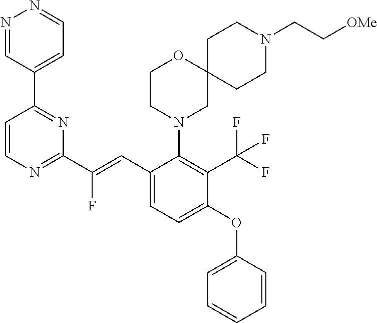 [SMILES: COCCN1CCC2(CC1)CN(c1c(/C=C(\F)c3nccc(-c4ccnnc4)n3)ccc(Oc3ccccc3)c1C(F)(F)F)CCO2]

The title compound was prepared according to the procedures described in Example 23, with 1-iodo-2-methoxyethane replacing (iodomethyl)cyclopropane in step 2. LC-MS calculated for C₃₄H₃₅F₄N₆O₃(M+H)⁺: m/z=651.3; found 651.5.

### Example 26. (Z)-4-(6-(2-Fluoro-2-(6-(pyridazin-4-yl)pyridin-2-yl)vinyl)-3-phenoxy-2-(trifluoromethyl)phenyl)-9-(2-methoxyethyl)-1-oxa-4,9-diazaspiro[5.5]undecane

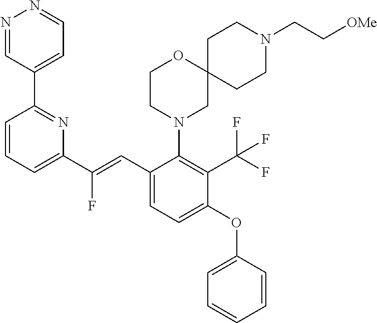 [SMILES: COCCN1CCC2(CC1)CN(c1c(/C=C(\F)c3cccc(-c4ccnnc4)n3)ccc(Oc3ccccc3)c1C(F)(F)F)CCO2]

The title compound was prepared according to the procedures described in Example 23, with 4-(6-chloropyridin-2-yl)pyridazine (Intermediate 2) replacing 4-(2-bromopyrimidin-4-yl)pyridazine (Intermediate 16) in step 1, and 1-iodo-2-methoxyethane replacing (iodomethyl)cyclopropane in step 2. LC-MS calculated for C₃₅H₃₆F₄N₅O₃ (M+H)⁺: m/z=650.3; found 650.4.

### Example 27. (Z)-9-(Cyclopropylmethyl)-4-(6-(2-fluoro-2-(6-(pyridazin-4-yl)pyridin-2-yl)vinyl)-3-phenoxy-2-(trifluoromethyl)phenyl)-1-oxa-4,9-diazaspiro[5.5]undecane

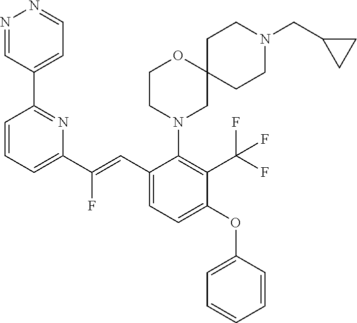 [SMILES: F/C(=C\c1ccc(Oc2ccccc2)c(C(F)(F)F)c1N1CCOC2(CCN(CC3CC3)CC2)C1)c1cccc(-c2ccnnc2)n1]

The title compound was prepared according to the procedures described in Example 23, with 4-(6-chloropyridin-2-yl)pyridazine (Intermediate 2) replacing 4-(2-bromopyrimidin-4-yl)pyridazine (Intermediate 16) in step 1. LC-MS calculated for C₃₆H₃₆F₄N₅O₂(M+H)⁺: m/z=646.3; found 646.4.

### Example 28. (Z)-9-(Cyclopropylmethyl)-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane

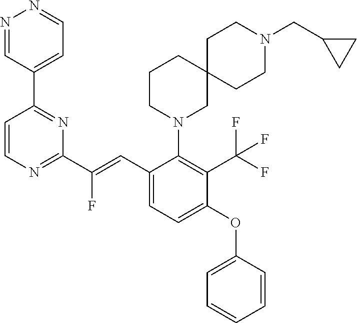 [SMILES: F/C(=C\c1ccc(Oc2ccccc2)c(C(F)(F)F)c1N1CCCC2(CCN(CC3CC3)CC2)C1)c1nccc(-c2ccnnc2)n1]

### Step 1. tert-Butyl (Z)-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane-9-carboxylate

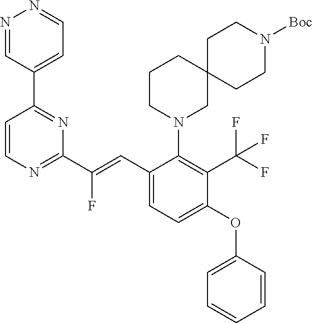 [SMILES: CC(C)(C)OC(=O)N1CCC2(CCCN(c3c(/C=C(\F)c4nccc(-c5ccnnc5)n4)ccc(Oc4ccccc4)c3C(F)(F)F)C2)CC1]

To a mixture of 4-(2-bromopyrimidin-4-yl)pyridazine (0.17 g, 0.72 mmol) (Intermediate 16) in 1,4-dioxane (4 mL) was added tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (0.40 g, 0.61 mmol) (Intermediate 19), cesium carbonate (0.59 g, 1.82 mmol), and [1,1′-bis(diphenylphosphino)ferrocene]dichloropalladium(II) (66 mg, 0.091 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.215 g, 51% yield). LC-MS calculated for C₃₇H₃₉F₄N₆O₃(M+H)⁺: m/z=691.3; found 691.3.

### Step 2. (Z)-9-(Cyclopropylmethyl)-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane

To a solution of tert-butyl (Z)-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane-9-carboxylate (0.20 g, 0.29 mmol) in DCM (2 mL) was added TFA/DCM 1:1 (2 mL) and the reaction mixture was stirred at 40° C. for 30 min. After cooling to rt, the solvent was concentrated under reduced pressure, and to the crude residue was added acetonitrile (5 mL), N-ethyl-N-isopropylpropan-2-amine (0.62 mL, 3.4 mmol) and (iodomethyl)cyclopropane (0.31 g, 1.7 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₆H₃₇F₄N₆O (M+H)⁺: m/z=645.3; found 645.3. ¹H NMR (600 MHz, DMSO-d₆) δ 10.09-9.98 (m, 1H), 9.59-9.54 (m, 1H), 9.33-9.13 (m, 2H), 8.48-8.41 (m, 1H), 8.38-8.34 (m, 1H), 8.10-8.04 (m, 1H), 7.99-7.82 (m, 1H), 7.50-7.42 (m, 2H), 7.27-7.21 (m, 1H), 7.13-7.07 (m, 2H), 6.94 (t, J=10.0 Hz, 1H), 3.47-3.27 (m, 3H), 3.27-3.17 (m, 1H), 3.17-2.79 (m, 6H), 2.29-2.14 (m, 1H), 2.04-1.78 (m, 3H), 1.73-1.42 (m, 4H), 1.12-0.92 (m, 1H), 0.69-0.54 (m, 2H), 0.44-0.23 (m, 2H).

### Example 29. (Z)-9-(Cyclopropylmethyl)-2-(6-(2-fluoro-2-(6-(pyridazin-4-yl)pyrazin-2-yl)vinyl)-3-phenoxy-2-(trifluoromethyl)phenyl)-2,9-diazaspiro[5.5]undecane

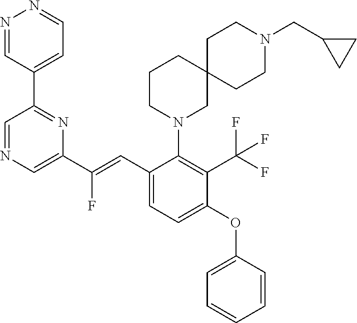 [SMILES: F/C(=C\c1ccc(Oc2ccccc2)c(C(F)(F)F)c1N1CCCC2(CCN(CC3CC3)CC2)C1)c1cncc(-c2ccnnc2)n1]

The title compound was prepared according to the procedures described in Example 28, with 4-(6-chloropyrazin-2-yl)pyridazine (Intermediate 1) replacing 4-(2-bromopyrimidin-4-yl)pyridazine (Intermediate 16) in step 1. LC-MS calculated for C₃₆H₃₇F₄N₆O (M+H)⁺: m/z=645.3; found 645.3. ¹H NMR (500 MHz, DMSO-d₆) δ 10.07-9.96 (m, 1H), 9.64-9.52 (m, 2H), 9.48-9.15 (m, 1H), 9.09 (s, 1H), 8.48-8.40 (m, 1H), 8.09-8.01 (m, 1H), 7.81-7.60 (m, 1H), 7.51-7.43 (m, 2H), 7.27-7.21 (m, 1H), 7.13-7.06 (m, 2H), 6.98-6.91 (m, 1H), 3.46-3.28 (m, 3H), 3.27-3.09 (m, 2H), 3.07-2.78 (m, 5H), 2.28-2.15 (m, 1H), 2.05-1.78 (m, 2H), 1.76-1.39 (m, 5H), 1.12-0.93 (m, 1H), 0.69-0.52 (m, 2H), 0.41-0.22 (m, 2H).

### Example 30. (Z)-9-Cyclobutyl-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane

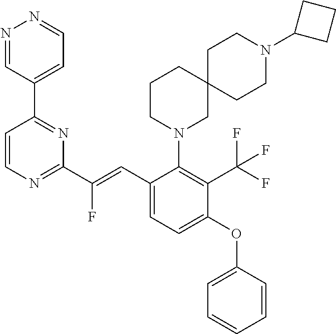 [SMILES: F/C(=C\c1ccc(Oc2ccccc2)c(C(F)(F)F)c1N1CCCC2(CCN(C3CCC3)CC2)C1)c1nccc(-c2ccnnc2)n1]

The title compound was prepared according to the procedures described in Example 28, with iodocyclobutane replacing (iodomethyl)cyclopropane in step 2 and the reaction temperature was heated up to 90° C. LC-MS calculated for C₃₆H₃₇F₄N₆O (M+H)⁺: m/z=645.3; found 645.4.

### Example 31. (Z)-2-(6-(2-Fluoro-2-(4-(pyridazin-4-yl)pyrimidin-2-yl)vinyl)-3-phenoxy-2-(trifluoromethyl)phenyl)-9-(2-methoxyethyl)-2,9-diazaspiro[5.5]undecane

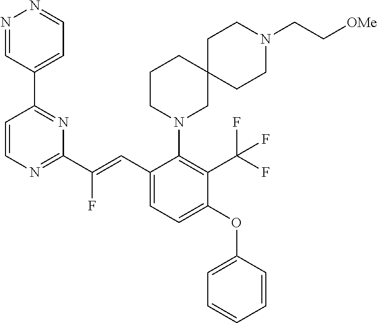 [SMILES: COCCN1CCC2(CCCN(c3c(/C=C(\F)c4nccc(-c5ccnnc5)n4)ccc(Oc4ccccc4)c3C(F)(F)F)C2)CC1]

The title compound was prepared according to the procedures described in Example 28, with 1-iodo-2-methoxyethane replacing (iodomethyl)cyclopropane in step 2. LC-MS calculated for C₃₅H₃₇F₄N₆O₂ (M+H)⁺: m/z=649.3; found 649.4. ¹H NMR (600 MHz, DMSO-d₆) δ 10.05-9.97 (m, 1H), 9.58-9.53 (m, 1H), 9.19-9.14 (m, 1H), 8.47-8.39 (m, 1H), 8.37-8.33 (m, 1H), 8.10-8.02 (m, 1H), 7.96-7.81 (m, 1H), 7.49-7.43 (m, 2H), 7.26-7.19 (m, 1H), 7.13-7.03 (m, 2H), 6.96-6.90 (m, 1H), 3.71-3.56 (m, 2H), 3.40-3.16 (m, 9H), 3.15-3.08 (m, 1H), 3.03-2.91 (m, 3H), 2.23-2.13 (m, 1H), 2.00-1.84 (m, 2H), 1.78-1.54 (m, 3H), 1.49-1.43 (m, 2H).

### Example 32. (Z)-9-Ethyl-2-(6-(2-fluoro-2-(4-(pyridazin-4-yl)pyrimidin-2-yl)vinyl)-3-((3-fluoropyridin-2-yl)oxy)-2-(trifluoromethyl)phenyl)-2,9-diazaspiro[5.5]undecane

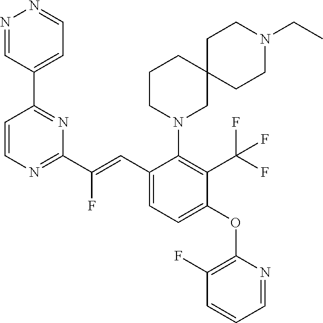 [SMILES: CCN1CCC2(CCCN(c3c(/C=C(\F)c4nccc(-c5ccnnc5)n4)ccc(Oc4ncccc4F)c3C(F)(F)F)C2)CC1]

The title compound was prepared according to the procedures described in Example 28, with tert-butyl (Z)-2-(6-(2-fluoro-2-(4,4,5,5-tetramethyl-1,3,2-dioxaborolan-2-yl)vinyl)-3-((3-fluoropyridin-2-yl)oxy)-2-(trifluoromethyl)phenyl)-2,9-diazaspiro[5.5]undecane-9-carboxylate (Intermediate 21) replacing tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (Intermediate 19) in step 1 and iodoethane replacing (iodomethyl)cyclopropane in step 2. LC-MS calculated for C₃₃H₃₃F₅N₇O (M+H)⁺: m/z=638.3; found 638.3. ¹H NMR (500 MHz, DMSO-d₆) δ 10.09-10.00 (m, 1H), 9.60-9.53 (m, 1H), 9.24-9.16 (m, 1H), 9.09-8.83 (m, 1H), 8.50-8.42 (m, 1H), 8.41-8.36 (m, 1H), 8.17-8.10 (m, 1H), 8.04-7.86 (m, 3H), 7.37-7.31 (m, 1H), 7.30-7.24 (m, 1H), 3.40-3.18 (m, 5H), 3.17-2.79 (m, 6H), 2.20-1.89 (m, 2H), 1.88-1.44 (m, 5H), 1.25-1.08 (m, 3H).

### Example 33. (Z)-9-Ethyl-2-(6-(2-fluoro-2-(6-(pyridazin-4-yl)pyridin-2-yl)vinyl)-3-((3-fluoropyridin-2-yl)oxy)-2-(trifluoromethyl)phenyl)-2,9-diazaspiro[5.5]undecane

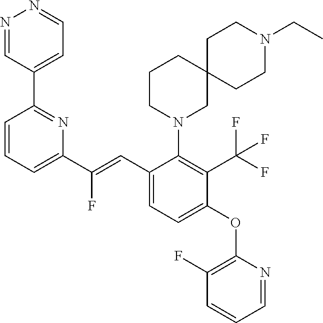 [SMILES: CCN1CCC2(CCCN(c3c(/C=C(\F)c4cccc(-c5ccnnc5)n4)ccc(Oc4ncccc4F)c3C(F)(F)F)C2)CC1]

The title compound was prepared according to the procedures described in Example 28, with 4-(6-chloropyridin-2-yl)pyridazine (Intermediate 2) replacing 4-(2-bromopyrimidin-4-yl)pyridazine (Intermediate 16) in step 1, tert-butyl (Z)-2-(6-(2-fluoro-2-(4,4,5,5-tetramethyl-1,3,2-dioxaborolan-2-yl)vinyl)-3-((3-fluoropyridin-2-yl)oxy)-2-(trifluoromethyl)phenyl)-2,9-diazaspiro[5.5]undecane-9-carboxylate (Intermediate 21) replacing tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (Intermediate 19) in step 1 and iodoethane replacing (iodomethyl)cyclopropane in step 2. LC-MS calculated for C₃₄H₃₄F₅N₆O (M+H)⁺: m/z=637.3; found 637.4. ¹H NMR (500 MHz, DMSO-d₆) δ 10.13-9.98 (m, 1H), 9.51-9.44 (m, 1H), 9.11-8.86 (m, 1H), 8.45-8.31 (m, 2H), 8.27-8.20 (m, 1H), 8.15-8.06 (m, 1H), 8.02-7.96 (m, 1H), 7.96-7.85 (m, 2H), 7.83-7.63 (m, 1H), 7.38-7.29 (m, 1H), 7.29-7.22 (m, 1H), 3.40-3.20 (m, 5H), 3.15-2.86 (m, 5H), 2.27-1.94 (m, 2H), 1.90-1.42 (m, 6H), 1.31-1.06 (m, 3H).

### Example 34. (Z)-9-Ethyl-4-(6-(2-fluoro-2-(6-(pyridazin-4-yl)pyrazin-2-yl)vinyl)-3-(3-fluorophenoxy)-2-(trifluoromethyl)phenyl)-1-oxa-4,9-diazaspiro[5.5]undecane

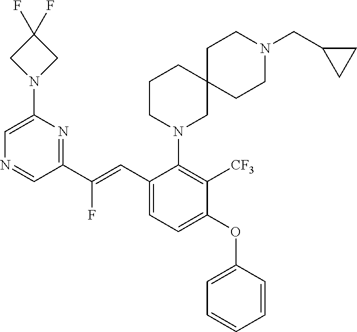 [SMILES: F/C(=C\c1ccc(Oc2ccccc2)c(C(F)(F)F)c1N1CCCC2(CCN(CC3CC3)CC2)C1)c1cncc(N2CC(F)(F)C2)n1]

### Step 1. tert-Butyl (Z)-4-(6-(2-fluoro-2-(6-(pyridazin-4-yl)pyrazin-2-yl)vinyl)-3-(3-fluorophenoxy)-2-(trifluoromethyl)phenyl)-1-oxa-4,9-diazaspiro[5.5]undecane-9-carboxylate

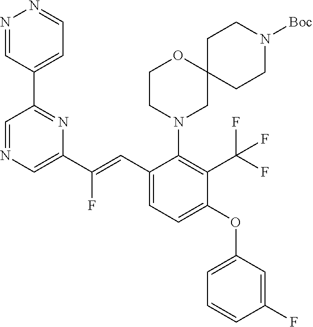 [SMILES: CC(C)(C)OC(=O)N1CCC2(CC1)CN(c1c(/C=C(\F)c3cncc(-c4ccnnc4)n3)ccc(Oc3cccc(F)c3)c1C(F)(F)F)CCO2]

To a mixture of 4-(6-chloropyrazin-2-yl)pyridazine (0.073 g, 0.38 mmol) (Intermediate 1) in 1,4-dioxane/water (6:1, 4 mL) was added tert-butyl (Z)-4-(6-(2-fluoro-2-(4,4,5,5-tetramethyl-1,3,2-dioxaborolan-2-yl)vinyl)-3-phenoxy-2-(trifluoromethyl)phenyl)-1-oxa-4,9-diazaspiro[5.5]undecane-9-carboxylate (0.26 g, 0.38 mmol) (Intermediate 22), cesium carbonate (0.369 g, 1.13 mmol), and [1,1′-bis(diphenylphosphino)ferrocene]dichloropalladium(II) (0.041 g, 0.057 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.19 g, 70% yield). LC-MS calculated for C₃₆H₃₆F₅N₆O₄ (M+H)⁺: m/z=711.3; found 711.3.

### Step 2. (Z)-9-(Cyclopropylmethyl)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane

To a solution of tert-butyl (Z)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane-9-carboxylate (0.10 g, 0.14 mmol) in MeOH (2 mL) was added 4 molar solution of HCl in 1,4-dioxane (0.5 mL, 2 mmol) and the reaction mixture was stirred at 40° C. for 30 mins. After cooling to rt, the solvent was concentrated under reduced pressure. To the crude residue was added acetonitrile (2 mL), N-ethyl-N-isopropylpropan-2-amine (0.62 mL, 3.4 mmol) and iodoethane (0.26 g, 1.70 mmol), and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₃H₃₂F₅N₆O₂(M+H)⁺: m/z=639.2; found 639.3. ¹H NMR (500 MHz, DMSO-d₆) δ 10.07-10.00 (m, 1H), 9.61-9.56 (m, 1H), 9.54-9.48 (m, 1H), 9.12 (s, 1H), 8.49-8.44 (m, 1H), 8.11-8.03 (m, 1H), 7.80-7.65 (m, 1H), 7.51-7.45 (m, 1H), 7.17-7.10 (m, 1H), 7.11-7.03 (m, 1H), 7.02-6.95 (m, 1H), 6.92-6.87 (m, 1H), 3.96-3.75 (m, 2H), 3.70-2.90 (m, 10H), 2.37-2.17 (m, 2H), 1.95-1.55 (m, 2H), 1.23-1.13 (m, 3H).

### Example 35. (Z)-9-Ethyl-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane

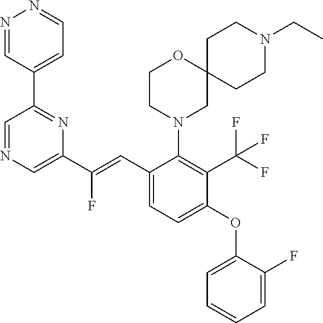 [SMILES: CCN1CCC2(CC1)CN(c1c(/C=C(\F)c3cncc(-c4ccnnc4)n3)ccc(Oc3ccccc3F)c1C(F)(F)F)CCO2]

### Step 1. tert-Butyl (Z)-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane-9-carboxylate

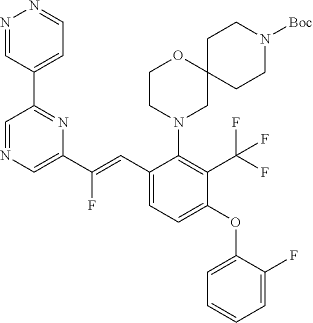 [SMILES: CC(C)(C)OC(=O)N1CCC2(CC1)CN(c1c(/C=C(\F)c3cncc(-c4ccnnc4)n3)ccc(Oc3ccccc3F)c1C(F)(F)F)CCO2]

To a mixture of 4-(6-chloropyrazin-2-yl)pyridazine (0.073 g, 0.38 mmol) (Intermediate 1) in 1,4-dioxane/water (6:1, 4 mL) was added tert-butyl (Z)-4-(6-(2-fluoro-2-(4,4,5,5-tetramethyl-1,3,2-dioxaborolan-2-yl)vinyl)-3-(2-fluorophenoxy)-2-(trifluoromethyl)phenyl)-1-oxa-4,9-diazaspiro[5.5]undecane-9-carboxylate (0.26 g, 0.38 mmol) (Intermediate 23), cesium carbonate (0.369 g, 1.13 mmol), and [1,1′-bis(diphenylphosphino)ferrocene]dichloropalladium(II) (0.041 g, 0.057 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.20 g, 75% yield). LC-MS calculated for C₃₆H₃₆F₅N₆O₄ (M+H)⁺: m/z=711.3; found 711.3.

### Step 2. (Z)-9-Ethyl-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane

To a solution of tert-butyl (Z)-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane-9-carboxylate (0.20 g, 0.29 mmol) in MeOH (2 mL) was added 4 molar solution of HCl in 1,4-dioxane (0.5 mL, 2 mmol) and the reaction mixture was stirred at 40° C. for 30 mins. After cooling to rt, the solvent was concentrated under reduced pressure. To the crude residue was added acetonitrile (2 mL), N-ethyl-N-isopropylpropan-2-amine (0.62 mL, 3.4 mmol) and iodoethane (0.26 g, 1.70 mmol), and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₃H₃₂F₅N₆O₂(M+H)⁺: m/z=639.2; found 639.2. ¹H NMR (500 MHz, DMSO-d₆) δ 10.00-9.96 (m, 1H), 9.49 (s, 1H), 9.45 (dd, J=5.4, 1.3 Hz, 1H), 9.05 (s, 1H), 8.39 (dd, J=5.4, 2.4 Hz, 1H), 7.99 (d, J=8.9 Hz, 1H), 7.80-7.68 (m, 1H), 7.44-7.38 (m, 1H), 7.33-7.20 (m, 3H), 6.90 (d, J=8.9 Hz, 1H), 3.91-3.77 (m, 2H), 3.18-3.03 (m, 4H), 2.40-2.17 (m, 6H), 2.09-1.81 (m, 2H), 1.61-1.52 (m, 2H), 0.94 (t, J=7.1 Hz, 3H).

### Example 36. (Z)-9-(Cyclopropylmethyl)-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane

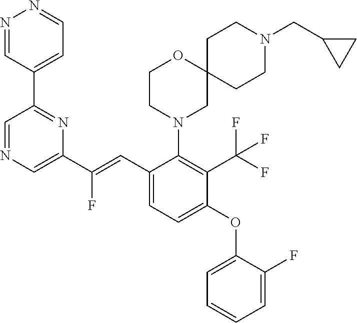 [SMILES: F/C(=C\c1ccc(Oc2ccccc2F)c(C(F)(F)F)c1N1CCOC2(CCN(CC3CC3)CC2)C1)c1cncc(-c2ccnnc2)n1]

The title compound was prepared according to the procedures described in Example 35, with (iodomethyl)cyclopropane replacing iodoethane in step 2. LC-MS calculated for C₃₅H₃₄F₅N₆O₂(M+H)⁺: m/z=665.3; found 665.3. ¹H NMR (500 MHz, DMSO-d₆) δ 10.01 (s, 1H), 9.53 (s, 1H), 9.48 (dd, J=5.4, 1.3 Hz, 1H), 9.39-9.15 (m, 1H), 9.08 (s, 1H), 8.43 (dd, J=5.4, 2.4 Hz, 1H), 8.00 (d, J=8.9 Hz, 1H), 7.77-7.63 (m, 1H), 7.46-7.38 (m, 1H), 7.35-7.26 (m, 2H), 7.26-7.19 (m, 1H), 6.96 (d, J=8.9 Hz, 1H), 3.88 (s, 2H), 3.53-2.90 (m, 10H), 2.43-2.14 (m, 2H), 2.02-1.63 (m, 2H), 1.10-0.97 (m, 1H), 0.68-0.59 (m, 2H), 0.48-0.28 (m, 2H).

### Example 37. (Z)-9-(Cyclopropylmethyl)-2-(6-(2-fluoro-2-(6-(pyridazin-4-yl)pyrazin-2-yl)vinyl)-3-(2-fluorophenoxy)-2-(trifluoromethyl)phenyl)-2,9-diazaspiro[5.5]undecane

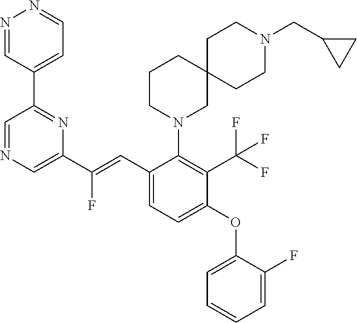 [SMILES: F/C(=C\c1ccc(Oc2ccccc2F)c(C(F)(F)F)c1N1CCCC2(CCN(CC3CC3)CC2)C1)c1cncc(-c2ccnnc2)n1]

The title compound was prepared according to the procedures described in Example 35, with tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (Intermediate 24) replacing tert-butyl (Z)-4-(6-(2-fluoro-2-(4,4,5,5-tetramethyl-1,3,2-dioxaborolan-2-yl)vinyl)-3-(2-fluorophenoxy)-2-(trifluoromethyl)phenyl)-1-oxa-4,9-diazaspiro[5.5]undecane-9-carboxylate (Intermediate 23) in step 1, and (iodomethyl)cyclopropane replacing iodoethane in step 2. LC-MS calculated for C₃₆H₃₆F₅N₆O (M+H)⁺: m/z=663.3; found 663.3. ¹H NMR (600 MHz, DMSO-d₆) δ 9.97 (dd, J=2.4, 1.2 Hz, 1H), 9.52 (s, 1H), 9.51 (dd, J=5.3, 1.3 Hz, 1H), 9.04 (s, 1H), 8.39 (dd, J=5.4, 2.3 Hz, 1H), 8.00 (d, J=8.9 Hz, 1H), 7.80-7.64 (m, 1H), 7.52-7.40 (m, 1H), 7.35-7.24 (m, 3H), 6.80 (d, J=8.9 Hz, 1H), 3.39-3.10 (m, 1H), 3.03-2.88 (m, 3H), 2.48-2.18 (m, 4H), 2.10 (d, J=6.5 Hz, 2H), 2.08-1.98 (m, 1H), 1.88-1.68 (m, 3H), 1.58-1.37 (m, 3H), 1.33-1.24 (m, 1H), 0.77-0.69 (m, 1H), 0.41-0.33 (m, 2H), 0.00 (dt, J=5.0, 2.6 Hz, 2H).

### Example 38. (Z)-9-(Cyclopropylmethyl)-2-(3-(2,4-difluorophenoxy)-6-(2-fluoro-2-(6-(pyridazin-4-yl)pyrazin-2-yl)vinyl)-2-(trifluoromethyl)phenyl)-2,9-diazaspiro[5.5]undecane

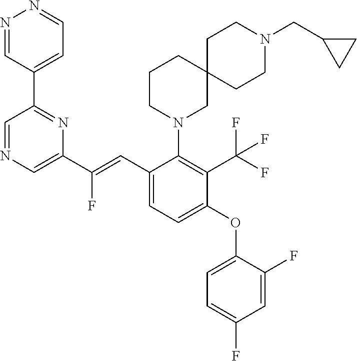 [SMILES: F/C(=C\c1ccc(Oc2ccc(F)cc2F)c(C(F)(F)F)c1N1CCCC2(CCN(CC3CC3)CC2)C1)c1cncc(-c2ccnnc2)n1]

### Step 1. tert-Butyl (Z)-2-(3-(2,4-difluorophenoxy)-6-(2-fluoro-2-(6-(pyridazin-4-yl)pyrazin-2-yl)vinyl)-2-(trifluoromethyl)phenyl)-2,9-diazaspiro[5.5]undecane-9-carboxylate

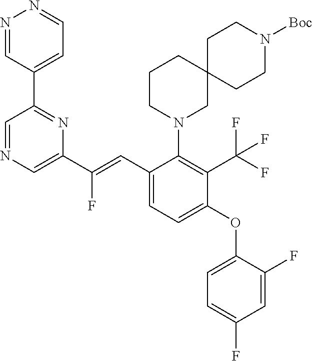 [SMILES: CC(C)(C)OC(=O)N1CCC2(CCCN(c3c(/C=C(\F)c4cncc(-c5ccnnc5)n4)ccc(Oc4ccc(F)cc4F)c3C(F)(F)F)C2)CC1]

To a mixture of 4-(6-chloropyrazin-2-yl)pyridazine (0.073 g, 0.38 mmol) (Intermediate 1) in 1,4-dioxane/water (6:1, 4 mL) was added tert-butyl (Z)-2-(3-(2,4-difluorophenoxy)-6-(2-fluoro-2-(4,4,5,5-tetramethyl-1,3,2-dioxaborolan-2-yl)vinyl)-2-(trifluoromethyl)phenyl)-2,9-diazaspiro[5.5]undecane-9-carboxylate (0.28 g, 0.38 mmol) (Intermediate 25) in 1,4-dioxane/water (6:1, 4 mL), cesium carbonate (0.369 g, 1.13 mmol), and [1,1′-bis(diphenylphosphino)ferrocene]dichloropalladium(II) (0.041 g, 0.057 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.16 g, 60% yield). LC-MS calculated for C₃₇H₃₇F₆N₆O₃ (M+H)⁺: m/z=727.3; found 727.2.

### Step 2. (Z)-9-(Cyclopropylmethyl)-2-(3-(2,4-difluorophenoxy)-6-(2-fluoro-2-(6-(pyridazin-4-yl)pyrazin-2-yl)vinyl)-2-(trifluoromethyl)phenyl)-2,9-diazaspiro[5.5]undecane

To a solution of tert-butyl (Z)-2-(3-(2,4-difluorophenoxy)-6-(2-fluoro-2-(6-(pyridazin-4-yl)pyrazin-2-yl)vinyl)-2-(trifluoromethyl)phenyl)-2,9-diazaspiro[5.5]undecane-9-carboxylate (0.11 g, 0.15 mmol) in MeOH (2 mL) was added 4 molar solution of HCl in 1,4-dioxane (0.5 mL, 2 mmol) and the reaction mixture was stirred at 40° C. for 30 mins. After cooling to rt, the solvent was concentrated under reduced pressure. To the crude residue was added acetonitrile (5 mL), N-ethyl-N-isopropylpropan-2-amine (0.31 mL, 1.7 mmol) and (iodomethyl)cyclopropane (0.16 g, 0.85 mmol), and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₆H₃₅F₆N₆O (M+H)⁺: m/z=681.3; found 681.3. ¹H NMR (600 MHz, DMSO-d₆) δ 10.08-9.94 (m, 1H), 9.58-9.51 (m, 2H), 9.09 (s, 2H), 8.50-8.38 (m, 1H), 8.07-7.97 (m, 1H), 7.77-7.50 (m, 2H), 7.41-7.30 (m, 1H), 7.24-7.15 (m, 1H), 6.95-6.84 (m, 1H), 3.45-3.32 (m, 2H), 3.32-3.03 (m, 3H), 3.03-2.84 (m, 5H), 2.21 (d, J=14.4 Hz, 1H), 2.04-1.84 (m, 2H), 1.84-1.41 (m, 5H), 1.07-0.94 (m, 1H), 0.67-0.54 (m, 2H), 0.41-0.23 (m, 2H).

### Example 39. (Z)-9-(Cyclopropylmethyl)-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane

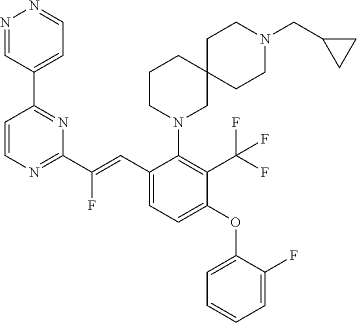 [SMILES: F/C(=C\c1ccc(Oc2ccccc2F)c(C(F)(F)F)c1N1CCCC2(CCN(CC3CC3)CC2)C1)c1nccc(-c2ccnnc2)n1]

### Step 1. tert-Butyl (Z)-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane-9-carboxylate

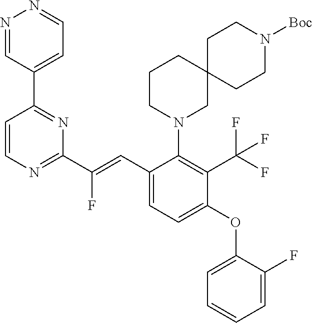 [SMILES: CC(C)(C)OC(=O)N1CCC2(CCCN(c3c(/C=C(\F)c4nccc(-c5ccnnc5)n4)ccc(Oc4ccccc4F)c3C(F)(F)F)C2)CC1]

To a mixture of 4-(2-bromopyrimidin-4-yl)pyridazine (0.090 g, 0.38 mmol) (Intermediate 16) in 1,4-dioxane/water (6:1, 4 mL) was added tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (0.26 g, 0.38 mmol) (Intermediate 24), cesium carbonate (0.369 g, 1.13 mmol), and [1,1′-bis(diphenylphosphino)ferrocene]dichloropalladium(II) (0.041 g, 0.057 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.15 g, 55% yield). LC-MS calculated for C₃₇H₃₈F₅N₆O₃ (M+H)⁺: m/z=709.3; found 709.2.

### Step 2. (Z)-9-(Cyclopropylmethyl)-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane

To a solution of tert-butyl (Z)-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane-9-carboxylate (0.10 g, 0.14 mmol) (from step 1) in MeOH (1 mL) was added 4 molar solution of HCl in 1,4-dioxane (0.5 mL, 2 mmol) and the reaction mixture was stirred at 40° C. for 30 mins. After cooling to rt, the solvent was concentrated under reduced pressure. To the crude residue was added acetonitrile (5 mL), N-ethyl-N-isopropylpropan-2-amine (0.31 mL, 1.7 mmol) and (iodomethyl)cyclopropane (0.16 g, 0.85 mmol), and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₆H₃₆F₅N₆O (M+H)⁺: m/z=663.3; found 663.4. ¹H NMR (500 MHz, DMSO-d₆) δ 10.07-9.98 (m, 1H), 9.59-9.53 (m, 1H), 9.21-9.16 (m, 1H), 9.06 (s, 1H), 8.48-8.40 (m, 1H), 8.40-8.30 (m, 1H), 8.09-8.02 (m, 1H), 7.99-7.80 (m, 1H), 7.50-7.42 (m, 1H), 7.39-7.20 (m, 3H), 6.92-6.85 (m, 1H), 3.51-2.82 (m, 10H), 2.29-2.14 (m, 1H), 2.09-1.42 (m, 7H), 1.12-0.93 (m, 1H), 0.71-0.52 (m, 2H), 0.40-0.25 (m, 2H).

### Example 40. (Z)-9-Ethyl-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane

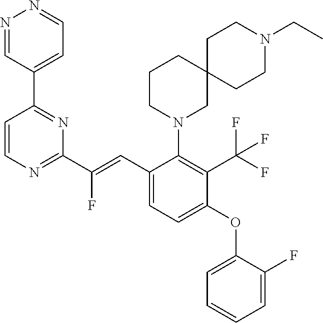 [SMILES: CCN1CCC2(CCCN(c3c(/C=C(\F)c4nccc(-c5ccnnc5)n4)ccc(Oc4ccccc4F)c3C(F)(F)F)C2)CC1]

The title compound was prepared according to the procedures described in Example 39, with iodoethane replacing (iodomethyl)cyclopropane in step 2. LC-MS calculated for C₃₄H₃₄F₅N₆O (M+H)⁺: m/z=637.3; found 637.3. ¹H NMR (500 MHz, DMSO-d₆) δ 10.09-9.96 (m, 1H), 9.60-9.51 (m, 1H), 9.20-9.14 (m, 1H), 9.11-8.87 (m, 1H), 8.47-8.39 (m, 1H), 8.38-8.30 (m, 1H), 8.11-8.00 (m, 1H), 7.97-7.79 (m, 1H), 7.53-7.41 (m, 1H), 7.38-7.21 (m, 3H), 6.95-6.81 (m, 1H), 3.42-2.81 (m, 10H), 2.27-2.12 (m, 1H), 2.09-1.40 (m, 7H), 1.25-1.12 (m, 3H).

### Example 41. (Z)-9-Ethyl-4-(6-(2-fluoro-2-(4-(pyridazin-4-yl)pyrimidin-2-yl)vinyl)-3-(2-fluorophenoxy)-2-(trifluoromethyl)phenyl)-1-oxa-4,9-diazaspiro[5.5]undecane

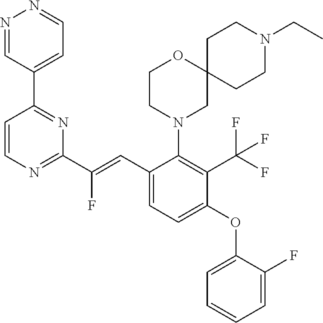 [SMILES: CCN1CCC2(CC1)CN(c1c(/C=C(\F)c3nccc(-c4ccnnc4)n3)ccc(Oc3ccccc3F)c1C(F)(F)F)CCO2]

The title compound was prepared according to the procedures described in Example 39, with tert-butyl (Z)-4-(6-(2-fluoro-2-(4,4,5,5-tetramethyl-1,3,2-dioxaborolan-2-yl)vinyl)-3-(2-fluorophenoxy)-2-(trifluoromethyl)phenyl)-1-oxa-4,9-diazaspiro[5.5]undecane-9-carboxylate (Intermediate 23) replacing tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (Intermediate 24) in step 1, and iodoethane replacing (iodomethyl)cyclopropane in step 2. LC-MS calculated for C₃₃H₃₂F₅N₆O₂(M+H)⁺: m/z=639.2; found 639.3. ¹H NMR (600 MHz, DMSO-d₆) δ 10.05-10.00 (m, 1H), 9.56-9.51 (m, 1H), 9.30-9.13 (m, 2H), 8.49-8.43 (m, 1H), 8.39-8.35 (m, 1H), 8.08-8.02 (m, 1H), 7.98-7.86 (m, 1H), 7.49-7.42 (m, 1H), 7.37-7.22 (m, 3H), 6.96-6.91 (m, 1H), 4.02-3.75 (m, 2H), 3.47-3.17 (m, 4H), 3.17-2.91 (m, 6H), 2.44-2.17 (m, 2H), 1.96-1.57 (m, 2H), 1.27-1.13 (m, 3H).

### Example 42. (Z)-9-(Cyclopropylmethyl)-4-(6-(2-fluoro-2-(4-(pyridazin-4-yl)pyrimidin-2-yl)vinyl)-3-(2-fluorophenoxy)-2-(trifluoromethyl)phenyl)-1-oxa-4,9-diazaspiro[5.5]undecane

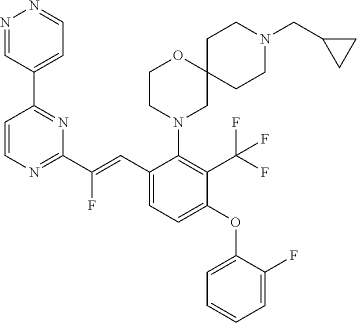 [SMILES: F/C(=C\c1ccc(Oc2ccccc2F)c(C(F)(F)F)c1N1CCOC2(CCN(CC3CC3)CC2)C1)c1nccc(-c2ccnnc2)n1]

The title compound was prepared according to the procedures described in Example 39, with tert-butyl (Z)-4-(6-(2-fluoro-2-(4,4,5,5-tetramethyl-1,3,2-dioxaborolan-2-yl)vinyl)-3-(2-fluorophenoxy)-2-(trifluoromethyl)phenyl)-1-oxa-4,9-diazaspiro[5.5]undecane-9-carboxylate (Intermediate 23) replacing tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (Intermediate 24) in step 1. LC-MS calculated for C₃₅H₃₄F₅N₆O₂(M+H)⁺: m/z=665.3; found 665.3. ¹H NMR (600 MHz, DMSO-d₆) δ 10.06-10.01 (m, 1H), 9.56-9.52 (m, 1H), 9.45-9.24 (m, 1H), 9.21-9.16 (m, 1H), 8.49-8.44 (m, 1H), 8.39-8.35 (m, 1H), 8.07-8.02 (m, 1H), 8.00-7.84 (m, 1H), 7.49-7.42 (m, 1H), 7.37-7.21 (m, 3H), 6.94 (d, J=8.9 Hz, 1H), 4.04-3.71 (m, 2H), 3.56-3.17 (m, 4H), 3.18-2.89 (m, 6H), 2.44-2.14 (m, 2H), 2.02-1.61 (m, 2H), 1.12-0.94 (m, 1H), 0.69-0.54 (m, 2H), 0.40-0.23 (m, 2H).

### Example 43. (S,Z)-(1-(6-(2-Fluoro-2-(4-(pyridazin-4-yl)pyrimidin-2-yl)vinyl)-3-phenoxy-2-(trifluoromethyl)phenyl)piperidin-3-yl)methanamine

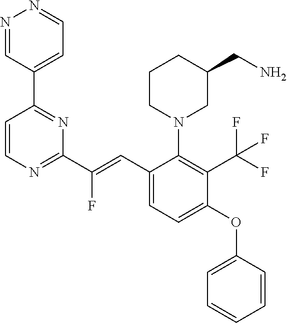 [SMILES: NC[C@@H]1CCCN(c2c(/C=C(\F)c3nccc(-c4ccnnc4)n3)ccc(Oc3ccccc3)c2C(F)(F)F)C1]

### Step 1. tert-Butyl (S,Z)-((1-(6-(2-fluoro-2-(4-(pyridazin-4-yl)pyrimidin-2-yl)vinyl)-3-phenoxy-2-(trifluoromethyl)phenyl)piperidin-3-yl)methyl)carbamate

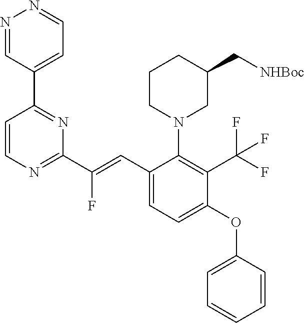 [SMILES: CC(C)(C)OC(=O)NC[C@@H]1CCCN(c2c(/C=C(\F)c3nccc(-c4ccnnc4)n3)ccc(Oc3ccccc3)c2C(F)(F)F)C1]

To a mixture of 4-(2-bromopyrimidin-4-yl)pyridazine (0.090 g, 0.38 mmol) (Intermediate 16) in 1,4-dioxane/water (6:1, 4 mL) was added tert-butyl (S,Z)-((1-(6-(2-fluoro-2-(4,4,5,5-tetramethyl-1,3,2-dioxaborolan-2-yl)vinyl)-3-phenoxy-2-(trifluoromethyl)phenyl)piperidin-3-yl)methyl)carbamate (0.20 g, 0.38 mmol) (Intermediate 26), cesium carbonate (0.369 g, 1.13 mmol), and [1,1′-bis(diphenylphosphino)ferrocene]dichloropalladium(II) (0.041 g, 0.057 mmol) and the reaction mixture was stirred at 90° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.13 g, 53% yield). LC-MS calculated for C₃₄H₃₅F₄N₆O₃(M+H)⁺: m/z=651.3; found 651.2.

### Step 2. (S,Z)-(1-(6-(2-Fluoro-2-(4-(pyridazin-4-yl)pyrimidin-2-yl)vinyl)-3-phenoxy-2-(trifluoromethyl)phenyl)piperidin-3-yl)methanamine

To a solution of tert-butyl (S,Z)-((1-(6-(2-fluoro-2-(4-(pyridazin-4-yl)pyrimidin-2-yl)vinyl)-3-phenoxy-2-(trifluoromethyl)phenyl)piperidin-3-yl)methyl)carbamate (0.026 g, 0.04 mmol) (from step 1) in MeOH (1 mL) was added 4 molar solution of HCl in 1,4-dioxane (0.2 mL, 0.8 mmol) and the reaction mixture was stirred at 40° C. for 30 mins. After cooling to rt, the solvent was concentrated under reduced pressure, and the crude mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₉H₂₇F₄N₆O (M+H)⁺: m/z=551.2; found 551.3. ¹H NMR (500 MHz, DMSO-d₆) δ 10.11-9.99 (m, 1H), 9.55 (d, J=5.4 Hz, 1H), 9.20-9.14 (m, 1H), 8.51-8.40 (m, 1H), 8.40-8.30 (m, 1H), 8.10 (d, J=8.9 Hz, 1H), 8.00-7.62 (m, 3H), 7.45 (q, J=7.3 Hz, 2H), 7.27-7.18 (m, 1H), 7.12-6.86 (m, 3H), 3.35-3.18 (m, 1H), 3.15-2.94 (m, 2H), 2.90-2.67 (m, 3H), 2.18-1.54 (m, 4H), 1.31-1.11 (m, 1H).

### Example 44. (Z)-2-(6-(2-(6-(3,3-Difluoroazetidin-1-yl)pyrazin-2-yl)-2-fluorovinyl)-3-(2-fluorophenoxy)-2-(trifluoromethyl)phenyl)-9-ethyl-2,9-diazaspiro[5.5]undecane

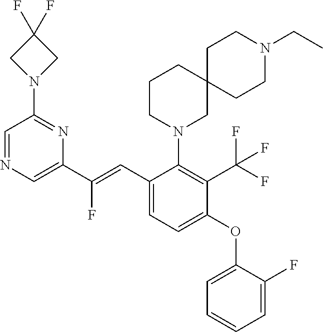 [SMILES: CCN1CCC2(CCCN(c3c(/C=C(\F)c4cncc(N5CC(F)(F)C5)n4)ccc(Oc4ccccc4F)c3C(F)(F)F)C2)CC1]

The title compound was prepared according to the procedures described in Example 39, with 2-bromo-6-(3,3-difluoroazetidin-1-yl)pyrazine (Intermediate 17) replacing 4-(2-bromopyrimidin-4-yl)pyridazine (Intermediate 16) in step 1 and iodoethane replacing (iodomethyl)cyclopropane in step 2. LC-MS calculated for C₃₃H₃₅F₇N₅O (M+H)⁺: m/z=650.3; found 650.3. ¹H NMR (500 MHz, DMSO-d₆) δ 9.05-8.80 (m, 1H), 8.27 (s, 1H), 8.16 (s, 1H), 7.96 (t, J=9.1 Hz, 1H), 7.48-7.18 (m, 5H), 6.87-6.79 (m, 1H), 4.65-4.49 (m, 4H), 3.41-2.76 (m, 10H), 2.19-1.74 (m, 4H), 1.70-1.39 (m, 4H), 1.25-1.12 (m, 3H).

### Example 45. (Z)-2-(2-(6-(2-Fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecan-9-yl)ethan-1-ol

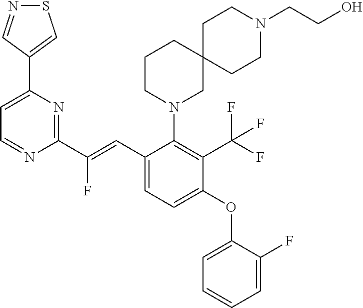 [SMILES: OCCN1CCC2(CCCN(c3c(/C=C(\F)c4nccc(-c5cnsc5)n4)ccc(Oc4ccccc4F)c3C(F)(F)F)C2)CC1]

### Step 1. tert-Butyl (Z)-2-(6-(2-fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecane-9-carboxylate

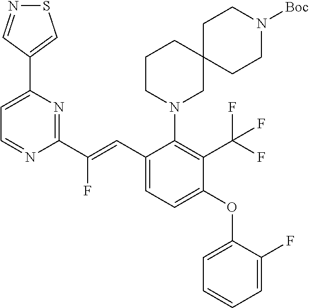 [SMILES: CC(C)(C)OC(=O)N1CCC2(CCCN(c3c(/C=C(\F)c4nccc(-c5cnsc5)n4)ccc(Oc4ccccc4F)c3C(F)(F)F)C2)CC1]

To a mixture of 4-(2-iodopyrimidin-4-yl)isothiazole (0.102 g, 0.35 mmol) (Intermediate 28) in 1,4-dioxane/H₂O (4:1, 10 mL) was added tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (0.20 g, 0.30 mmol) (Intermediate 24), cesium carbonate (0.29 g, 0.88 mmol), and chloro(2-dicyclohexylphosphino-2′,4′,6′-triisopropyl-1,1′-biphenyl)[2-(2′-amino-1,1′-biphenyl)]palladium(II) (34 mg, 0.044 mmol) and the reaction mixture was stirred at 100° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.18 g, 86% yield). LC-MS calculated for C₃₆H₃₇F₅N₅O₃S (M+H)⁺: m/z=714.3; found 714.4.

### Step 2. (Z)-2-(2-(6-(2-Fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecan-9-yl)ethan-1-ol

To a mixture of tert-butyl (Z)-2-(6-(2-fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecane-9-carboxylate (0.18 g, 0.25 mmol) in MeOH (2 mL) was added a 4 molar solution of HCl in 1,4-dioxane (2 mL, 8 mmol) and the reaction mixture was stirred at 40° C. for 30 min. After cooling to rt, the solvent was concentrated under reduced pressure, and to the crude residue was added acetonitrile (5 mL), N-ethyl-N-isopropylpropan-2-amine (0.46 mL, 2.5 mmol) and tert-butyl(2-iodoethoxy)dimethylsilane (358 mg, 1.25 mmol) and the reaction mixture was stirred at 40° C. for 30 mins. After cooling to rt, the solvent was concentrated under reduced pressure, and to the crude residue was added DMF (5 mL), cesium fluoride (114 mg, 0.75 mmol) and the reaction mixture was stirred at 80° C. for 1 hour. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₃H₃₃F₅N₅O₂S (M+H)⁺: m/z=658.2; found 658.3. ¹H NMR (500 MHz, DMSO-d₆) δ 9.92-9.84 (m, 1H), 9.35-9.28 (m, 1H), 9.03-8.96 (m, 1H), 8.07-7.97 (m, 2H), 7.87-7.72 (m, 1H), 7.49-7.41 (m, 1H), 7.36-7.19 (m, 3H), 6.90-6.83 (m, 1H), 3.75-3.59 (m, 2H), 3.45-3.25 (m, 2H), 3.25-3.04 (m, 5H), 3.04-2.89 (m, 4H), 2.21-2.00 (m, 1H), 2.00-1.81 (m, 2H), 1.78-1.52 (m, 3H), 1.52-1.36 (m, 2H).

### Example 46. (Z)-4-(2-(2-(2-(9-Ethyl-2,9-diazaspiro[5.5]undecan-2-yl)-4-(2-fluorophenoxy)-3-(trifluoromethyl)phenyl)-1-fluorovinyl)pyrimidin-4-yl)isothiazole

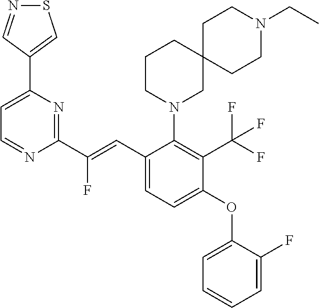 [SMILES: CCN1CCC2(CCCN(c3c(/C=C(\F)c4nccc(-c5cnsc5)n4)ccc(Oc4ccccc4F)c3C(F)(F)F)C2)CC1]

### Step 1. tert-Butyl (Z)-2-(6-(2-fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecane-9-carboxylate

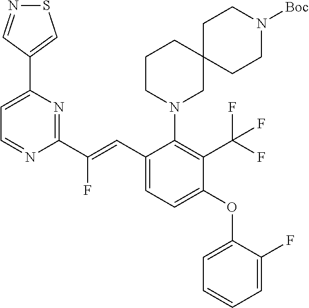 [SMILES: CC(C)(C)OC(=O)N1CCC2(CCCN(c3c(/C=C(\F)c4nccc(-c5cnsc5)n4)ccc(Oc4ccccc4F)c3C(F)(F)F)C2)CC1]

To a mixture of 4-(2-iodopyrimidin-4-yl)isothiazole (0.102 g, 0.35 mmol) (Intermediate 28) in 1,4-dioxane/H₂O (4:1, 10 mL) was added tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (0.20 g, 0.30 mmol) (Intermediate 24), cesium carbonate (0.29 g, 0.88 mmol), and chloro(2-dicyclohexylphosphino-2′,4′,6′-triisopropyl-1,1′-biphenyl)[2-(2′-amino-1,1′-biphenyl)]palladium(II) (34 mg, 0.044 mmol) and the reaction mixture was stirred at 100° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.18 g, 86% yield). LC-MS calculated for C₃₆H₃₇F₅N₅O₃S (M+H)⁺: m/z=714.3; found 714.4.

### Step 2. (Z)-4-(2-(2-(2-(9-Ethyl-2,9-diazaspiro[5.5]undecan-2-yl)-4-(2-fluorophenoxy)-3-(trifluoromethyl)phenyl)-1-fluorovinyl)pyrimidin-4-yl)isothiazole

To a mixture of tert-butyl (Z)-2-(6-(2-fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecane-9-carboxylate (0.18 g, 0.25 mmol) in MeOH (2 mL) was added a 4 molar solution of HCl in 1,4-dioxane (2 mL, 8 mmol) and the reaction mixture was stirred at 40° C. for 30 min. After cooling to rt, the solvent was concentrated under reduced pressure, and to the crude residue was added acetonitrile (5 mL), N-ethyl-N-isopropylpropan-2-amine (0.46 mL, 2.5 mmol) and iodoethane (195 mg, 1.25 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₃H₃₃F₅N₅OS (M+H)⁺: m/z=642.2; found 642.4. ¹H NMR (500 MHz, DMSO-d₆) δ 9.92-9.86 (m, 1H), 9.35-9.28 (m, 1H), 9.02-8.96 (m, 1H), 8.09-7.97 (m, 2H), 7.88-7.72 (m, 1H), 7.49-7.41 (m, 1H), 7.36-7.19 (m, 3H), 6.90-6.82 (m, 1H), 3.39-3.25 (m, 2H), 3.24-3.16 (m, 2H), 3.16-2.96 (m, 4H), 2.95-2.81 (m, 2H), 2.24-1.76 (m, 4H), 1.69-1.42 (m, 4H), 1.25-1.11 (m, 3H).

### Example 47. (Z)-4-(6-(2-(2-(9-(Cyclopropylmethyl)-2,9-diazaspiro[5.5]undecan-2-yl)-4-(2,3-difluorophenoxy)-3-(trifluoromethyl)phenyl)-1-fluorovinyl)pyrazin-2-yl)isothiazole

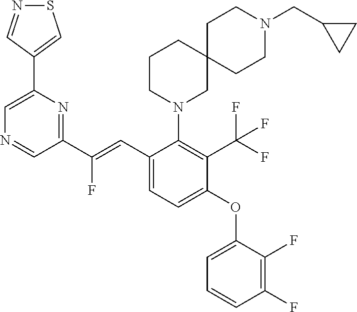 [SMILES: F/C(=C\c1ccc(Oc2cccc(F)c2F)c(C(F)(F)F)c1N1CCCC2(CCN(CC3CC3)CC2)C1)c1cncc(-c2cnsc2)n1]

The title compound was prepared according to the procedures described in Example 46, with 4-(6-chloropyrazin-2-yl)isothiazole (Intermediate 27) replacing 4-(2-iodopyrimidin-4-yl)isothiazole (Intermediate 28) and tert-butyl (Z)-2-(3-(2,3-difluorophenoxy)-6-(2-fluoro-2-(4,4,5,5-tetramethyl-1,3,2-dioxaborolan-2-yl)vinyl)-2-(trifluoromethyl)phenyl)-2,9-diazaspiro[5.5]undecane-9-carboxylate (Intermediate 29) replacing tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (0.20 g, 0.30 mmol) (Intermediate 24) in step 1 and (iodomethyl)cyclopropane replacing iodoethane in step 2. LC-MS calculated for C₃₅H₃₄F₆N₅OS (M+H)⁺: m/z=686.2; found 686.3. ¹H NMR (500 MHz, DMSO-d₆) δ 9.85-9.79 (m, 1H), 9.36-9.26 (m, 2H), 8.90 (s, 1H), 8.06-7.93 (m, 1H), 7.66-7.51 (m, 1H), 7.38-7.24 (m, 2H), 7.08-6.99 (m, 2H), 3.40-3.17 (m, 3H), 3.12-2.69 (m, 8H), 2.21-2.11 (m, 1H), 2.02-1.74 (m, 3H), 1.69-1.40 (m, 3H), 1.03-0.92 (m, 1H), 0.65-0.51 (m, 2H), 0.41-0.20 (m, 2H).

### Example 48. (Z)-2-(3-(Cyclohexyloxy)-6-(2-fluoro-2-(6-(pyridazin-4-yl)pyrazin-2-yl)vinyl)-2-(trifluoromethyl)phenyl)-9-(cyclopropylmethyl)-2,9-diazaspiro[5.5]undecane

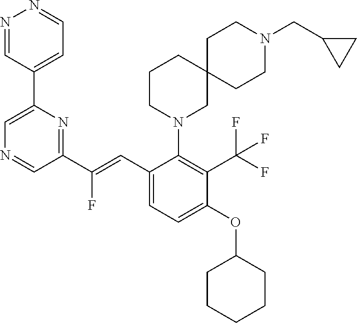 [SMILES: F/C(=C\c1ccc(OC2CCCCC2)c(C(F)(F)F)c1N1CCCC2(CCN(CC3CC3)CC2)C1)c1cncc(-c2ccnnc2)n1]

### Step 1. tert-Butyl (Z)-2-(3-(cyclohexyloxy)-6-(2-fluoro-2-(6-(pyridazin-4-yl)pyrazin-2-yl)vinyl)-2-(trifluoromethyl)phenyl)-2,9-diazaspiro[5.5]undecane-9-carboxylate

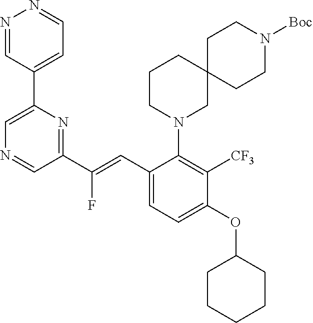 [SMILES: CC(C)(C)OC(=O)N1CCC2(CCCN(c3c(/C=C(\F)c4cncc(-c5ccnnc5)n4)ccc(OC4CCCCC4)c3C(F)(F)F)C2)CC1]

To a mixture of tert-butyl (Z)-2-(6-(2-fluoro-2-(6-(pyridazin-4-yl)pyrazin-2-yl)vinyl)-3-hydroxy-2-(trifluoromethyl)phenyl)-2,9-diazaspiro[5.5]undecane-9-carboxylate (0.600 g, 0.488 mmol) (Intermediate 30) in DMF (5 mL) was added iodocyclohexane (410 mg, 1.95 mmol) and potassium carbonate (404 mg, 2.92 mmol) and the reaction mixture was stirred at 120° C. for 1 hour before the reaction mixture was poured into water. The mixture was extracted with EtOAc, dried over sodium sulfate, and concentrated in vacuo. The crude residue was purified by flash column chromatography (SiO₂, EtOAc/Hexanes) to afford the desired product (0.32 g, 47% yield). LC-MS calculated for C₃₇H₄₅F₄N₆O₃(M+H)⁺: m/z=697.3; found 697.3.

### Step 2. (Z)-2-(3-(Cyclohexyloxy)-6-(2-fluoro-2-(6-(pyridazin-4-yl)pyrazin-2-yl)vinyl)-2-(trifluoromethyl)phenyl)-9-(cyclopropylmethyl)-2,9-diazaspiro[5.5]undecane

To a mixture of tert-butyl (Z)-2-(3-(cyclohexyloxy)-6-(2-fluoro-2-(6-(pyridazin-4-yl)pyrazin-2-yl)vinyl)-2-(trifluoromethyl)phenyl)-2,9-diazaspiro[5.5]undecane-9-carboxylate (0.17 g, 0.25 mmol) in MeOH (2 mL) was added a 4 molar solution of HCl in 1,4-dioxane (2 mL, 8 mmol) and the reaction mixture was stirred at 40° C. for 30 min. After cooling to rt, the solvent was concentrated under reduced pressure, and to the crude residue was added acetonitrile (5 mL), N-ethyl-N-isopropylpropan-2-amine (0.44 mL, 2.5 mmol) and (iodomethyl)cyclopropane (228 mg, 1.26 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₆H₄₃F₄N₆O (M+H)⁺: m/z=651.3; found 651.4. ¹H NMR (600 MHz, DMSO-d₆) δ 10.03-9.95 (m, 1H), 9.56-9.49 (m, 2H), 9.07 (s, 1H), 8.47-8.37 (m, 1H), 8.05-7.97 (m, 1H), 7.74-7.57 (m, 1H), 7.27-7.20 (m, 1H), 4.64-4.57 (m, 1H), 3.43-3.22 (m, 2H), 3.18-2.72 (m, 8H), 2.27-2.19 (m, 1H), 2.08-1.81 (m, 4H), 1.79-1.27 (m, 13H), 1.04-0.91 (m, 1H), 0.64-0.50 (m, 2H), 0.38-0.22 (m, 2H).

### Example 49. (Z)-5-(6-(2-(2-(9-(Cyclopropylmethyl)-2,9-diazaspiro[5.5]undecan-2-yl)-4-(2,3-difluorophenoxy)-3-(trifluoromethyl)phenyl)-1-fluorovinyl)pyrazin-2-yl)isothiazole

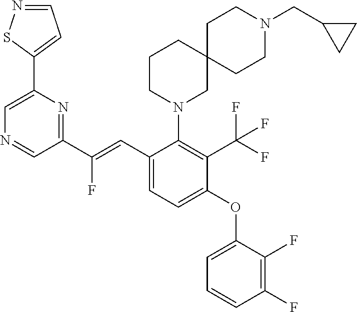 [SMILES: F/C(=C\c1ccc(Oc2cccc(F)c2F)c(C(F)(F)F)c1N1CCCC2(CCN(CC3CC3)CC2)C1)c1cncc(-c2ccns2)n1]

### Step 1. tert-Butyl (Z)-2-(6-(2-(6-chloropyrazin-2-yl)-2-fluorovinyl)-3-(2,3-difluorophenoxy)-2-(trifluoromethyl)phenyl)-2,9-diazaspiro[5.5]undecane-9-carboxylate

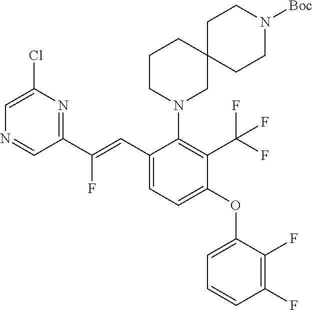 [SMILES: CC(C)(C)OC(=O)N1CCC2(CCCN(c3c(/C=C(\F)c4cncc(Cl)n4)ccc(Oc4cccc(F)c4F)c3C(F)(F)F)C2)CC1]

To a mixture of 2,6-dichloropyrazine (90 mg, 0.60 mmol) in 1,4-dioxane/H₂O (4:1, 10 mL) was added tert-butyl (Z)-2-(3-(2,3-difluorophenoxy)-6-(2-fluoro-2-(4,4,5,5-tetramethyl-1,3,2-dioxaborolan-2-yl)vinyl)-2-(trifluoromethyl)phenyl)-2,9-diazaspiro[5.5]undecane-9-carboxylate (0.30 g, 0.43 mmol) (Intermediate 29), cesium carbonate (0.42 g, 1.3 mmol), and 1,1′-bis(diphenylphosphino)ferrocene]dichloropalladium(II) (47 mg, 0.064 mmol) and the reaction mixture was stirred at 70° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (0.16 g, 54% yield). LC-MS calculated for C₃₃H₃₄ClF₆N₄O₃ (M+H)⁺: m/z=683.2; found 683.3.

### Step 2. tert-Butyl (Z)-2-(3-(2,3-difluorophenoxy)-6-(2-fluoro-2-(6-(isothiazol-5-yl)pyrazin-2-yl)vinyl)-2-(trifluoromethyl)phenyl)-2,9-diazaspiro[5.5]undecane-9-carboxylate

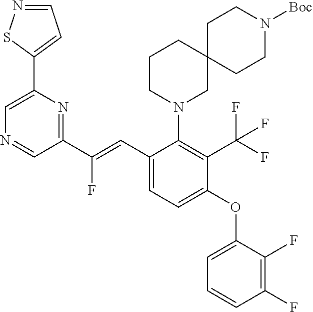 [SMILES: CC(C)(C)OC(=O)N1CCC2(CCCN(c3c(/C=C(\F)c4cncc(-c5ccns5)n4)ccc(Oc4cccc(F)c4F)c3C(F)(F)F)C2)CC1]

To a mixture of 5-(4,4,5,5-tetramethyl-1,3,2-dioxaborolan-2-yl)isothiazole (28 mg, 0.13 mmol) in 1,4-dioxane/H₂O (4:1, 10 mL) was added tert-butyl (Z)-2-(6-(2-(6-chloropyrazin-2-yl)-2-fluorovinyl)-3-(2,3-difluorophenoxy)-2-(trifluoromethyl)phenyl)-2,9-diazaspiro[5.5]undecane-9-carboxylate (70 mg, 0.10 mmol), cesium carbonate (0.10 g, 0.31 mmol), and 1,1′-bis(diphenylphosphino)ferrocene]dichloropalladium(II) (11 mg, 0.015 mmol) and the reaction mixture was stirred at 100° C. for 1 hour. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (48 mg, 64% yield). LC-MS calculated for C₃₆H₃₆F₆N₅O₃S (M+H)⁺: m/z=732.2; found 732.3.

### Step 3. (Z)-5-(6-(2-(2-(9-(Cyclopropylmethyl)-2,9-diazaspiro[5.5]undecan-2-yl)-4-(2,3-difluorophenoxy)-3-(trifluoromethyl)phenyl)-1-fluorovinyl)pyrazin-2-yl)isothiazole

To a mixture of tert-butyl (Z)-2-(3-(2,3-difluorophenoxy)-6-(2-fluoro-2-(6-(isothiazol-5-yl)pyrazin-2-yl)vinyl)-2-(trifluoromethyl)phenyl)-2,9-diazaspiro[5.5]undecane-9-carboxylate (48 mg, 0.10 mmol) in MeOH (2 mL) was added a 4 molar solution of HCl in 1,4-dioxane (2 mL, 8 mmol) and the reaction mixture was stirred at 40° C. for 30 min. After cooling to rt, the solvent was concentrated under reduced pressure, and to the crude residue was added acetonitrile (5 mL), N-ethyl-N-isopropylpropan-2-amine (0.18 mL, 1.0 mmol) and (iodomethyl)cyclopropane (90 mg, 0.50 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₅H₃₄F₆N₅OS (M+H)⁺: m/z=686.2; found 686.4.

### Example 50. (Z)-2-(3-(Cyclohexyloxy)-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane

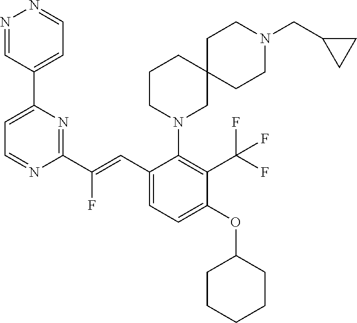 [SMILES: F/C(=C\c1ccc(OC2CCCCC2)c(C(F)(F)F)c1N1CCCC2(CCN(CC3CC3)CC2)C1)c1nccc(-c2ccnnc2)n1]

The title compound was prepared according to the procedures described in Example 48, with tert-butyl (Z)-2-(6-(2-fluoro-2-(4-(pyridazin-4-yl)pyrimidin-2-yl)vinyl)-3-hydroxy-2-(trifluoromethyl)phenyl)-2,9-diazaspiro[5.5]undecane-9-carboxylate (Intermediate 31) replacing tert-butyl (Z)-2-(6-(2-fluoro-2-(6-(pyridazin-4-yl)pyrazin-2-yl)vinyl)-3-hydroxy-2-(trifluoromethyl)phenyl)-2,9-diazaspiro[5.5]undecane-9-carboxylate (Intermediate 30) in step 1. LC-MS calculated for C₃₆H₄₃F₄N₆O (M+H)⁺: m/z=651.3; found 651.5.

### Example 51. (Z)-2-(3-(Cyclopentyloxy)-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane

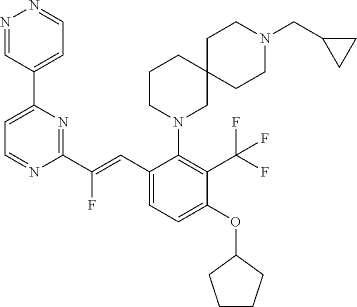 [SMILES: F/C(=C\c1ccc(OC2CCCC2)c(C(F)(F)F)c1N1CCCC2(CCN(CC3CC3)CC2)C1)c1nccc(-c2ccnnc2)n1]

The title compound was prepared according to the procedures described in Example 48, with tert-butyl (Z)-2-(6-(2-fluoro-2-(4-(pyridazin-4-yl)pyrimidin-2-yl)vinyl)-3-hydroxy-2-(trifluoromethyl)phenyl)-2,9-diazaspiro[5.5]undecane-9-carboxylate (Intermediate 31) replacing tert-butyl (Z)-2-(6-(2-fluoro-2-(6-(pyridazin-4-yl)pyrazin-2-yl)vinyl)-3-hydroxy-2-(trifluoromethyl)phenyl)-2,9-diazaspiro[5.5]undecane-9-carboxylate (Intermediate 30) and bromocyclopentane replacing iodocyclohexane in step 1. LC-MS calculated for C₃₅H₄₁F₄N₆O (M+H)⁺: m/z=637.3; found 637.4.

### Example 52. (Z)-2-(3-Cyclobutoxy-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane

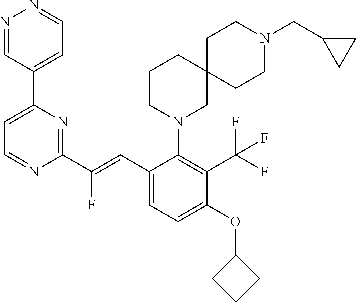 [SMILES: F/C(=C\c1ccc(OC2CCC2)c(C(F)(F)F)c1N1CCCC2(CCN(CC3CC3)CC2)C1)c1nccc(-c2ccnnc2)n1]

The title compound was prepared according to the procedures described in Example 48, with tert-butyl (Z)-2-(6-(2-fluoro-2-(4-(pyridazin-4-yl)pyrimidin-2-yl)vinyl)-3-hydroxy-2-(trifluoromethyl)phenyl)-2,9-diazaspiro[5.5]undecane-9-carboxylate (Intermediate 31) replacing tert-butyl (Z)-2-(6-(2-fluoro-2-(6-(pyridazin-4-yl)pyrazin-2-yl)vinyl)-3-hydroxy-2-(trifluoromethyl)phenyl)-2,9-diazaspiro[5.5]undecane-9-carboxylate (Intermediate 30) and bromocyclobutane replacing iodocyclohexane in step 1. LC-MS calculated for C₃₄H₃₉F₄N₆O (M+H)⁺: m/z=623.3; found 623.4.

### Example 53. (Z)-2-(3-(Cyclopentyloxy)-6-(2-fluoro-2-(4-(pyridazin-4-yl)pyrimidin-2-yl)vinyl)-2-(trifluoromethyl)phenyl)-9-ethyl-2,9-diazaspiro[5.5]undecane

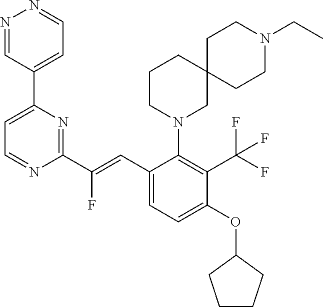 [SMILES: CCN1CCC2(CCCN(c3c(/C=C(\F)c4nccc(-c5ccnnc5)n4)ccc(OC4CCCC4)c3C(F)(F)F)C2)CC1]

### Step 1. tert-Butyl (Z)-2-(3-(cyclopentyloxy)-6-(2-fluoro-2-(4-(pyridazin-4-yl)pyrimidin-2-yl)vinyl)-2-(trifluoromethyl)phenyl)-2,9-diazaspiro[5.5]undecane-9-carboxylate

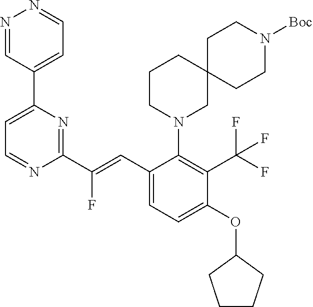 [SMILES: CC(C)(C)OC(=O)N1CCC2(CCCN(c3c(/C=C(\F)c4nccc(-c5ccnnc5)n4)ccc(OC4CCCC4)c3C(F)(F)F)C2)CC1]

To a mixture of tert-butyl (Z)-2-(6-(2-fluoro-2-(4-(pyridazin-4-yl)pyrimidin-2-yl)vinyl)-3-hydroxy-2-(trifluoromethyl)phenyl)-2,9-diazaspiro[5.5]undecane-9-carboxylate (40 mg, 0.065 mmol) (Intermediate 31) in DMF (2 mL) was added bromocyclobutane (17 mg, 0.13 mmol) and potassium carbonate (27 mg, 0.20 mmol) and the reaction mixture was stirred at 120° C. for 1 hour before the reaction mixture was poured into water. The mixture was extracted with EtOAc, dried over sodium sulfate, and concentrated in vacuo. The crude residue was purified by flash column chromatography (SiO₂, EtOAc/Hexanes) to afford the desired product (30 mg, 69% yield). LC-MS calculated for C₃₆H₄₃F₄N₆O₃(M+H)⁺: m/z=683.3; found 683.5.

### Step 2. (Z)-2-(3-(Cyclopentyloxy)-6-(2-fluoro-2-(4-(pyridazin-4-yl)pyrimidin-2-yl)vinyl)-2-(trifluoromethyl)phenyl)-9-ethyl-2,9-diazaspiro[5.5]undecane

To a mixture of tert-butyl (Z)-2-(3-(cyclohexyloxy)-6-(2-fluoro-2-(6-(pyridazin-4-yl)pyrazin-2-yl)vinyl)-2-(trifluoromethyl)phenyl)-2,9-diazaspiro[5.5]undecane-9-carboxylate (15 mg, 0.026 mmol) in MeOH (2 mL) was added a 4 molar solution of HCl in 1,4-dioxane (2 mL, 80 mmol) and the reaction mixture was stirred at 40° C. for 30 min. After cooling to rt, the solvent was concentrated under reduced pressure, and to the crude residue was added acetonitrile (2 mL), N-ethyl-N-isopropylpropan-2-amine (0.046 mL, 0.26 mmol) and iodoethane (21 mg, 0.13 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₃H₃₉F₄N₆O (M+H)⁺: m/z=611.3; found 611.4.

### Example 54. (Z)-2-(3-(Bicyclo[3.1.1]heptan-3-yloxy)-6-(2-fluoro-2-(4-(pyridazin-4-yl)pyrimidin-2-yl)vinyl)-2-(trifluoromethyl)phenyl)-9-ethyl-2,9-diazaspiro[5.5]undecane

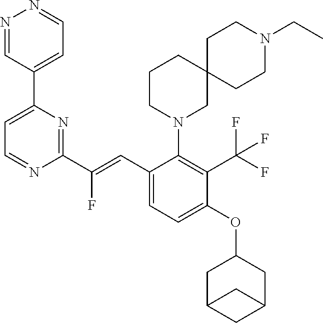 [SMILES: CCN1CCC2(CCCN(c3c(/C=C(\F)c4nccc(-c5ccnnc5)n4)ccc(OC4CC5CC(C5)C4)c3C(F)(F)F)C2)CC1]

### Step 1. tert-Butyl (Z)-2-(3-(bicyclo[3.1.1]heptan-3-yloxy)-6-(2-fluoro-2-(4-(pyridazin-4-yl)pyrimidin-2-yl)vinyl)-2-(trifluoromethyl)phenyl)-2,9-diazaspiro[5.5]undecane-9-carboxylate

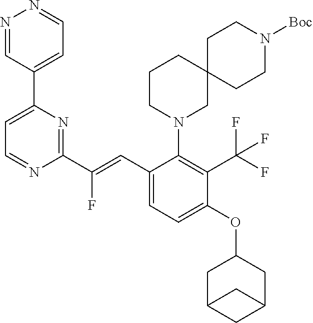 [SMILES: CC(C)(C)OC(=O)N1CCC2(CCCN(c3c(/C=C(\F)c4nccc(-c5ccnnc5)n4)ccc(OC4CC5CC(C5)C4)c3C(F)(F)F)C2)CC1]

To a mixture of bicyclo[3.1.1]heptan-3-ol (100 mg, 0.891 mmol) in DCM (3 mL) was added N-ethyl-N-isopropylpropan-2-amine (0.40 mL, 2.2 mmol) and methanesulfonyl chloride (0.10 ml, 1.3 mmol) at 0° C. and the reaction mixture was stirred at 0° C. for 1 hour before the reaction mixture was poured into water. The mixture was extracted with DCM, dried over sodium sulfate, and concentrated in vacuo. The crude residue was directly used without further purification. To a mixture of the crude material in DMF (5 mL) was added tert-butyl (Z)-2-(6-(2-fluoro-2-(4-(pyridazin-4-yl)pyrimidin-2-yl)vinyl)-3-hydroxy-2-(trifluoromethyl)phenyl)-2,9-diazaspiro[5.5]undecane-9-carboxylate (150 mg, 0.244 mmol) (Intermediate 31) and potassium carbonate (101 mg, 0.732 mmol) and the reaction mixture was stirred at 120° C. for 1 hour before the reaction mixture was poured into water. The mixture was extracted with EtOAc, dried over sodium sulfate, and concentrated in vacuo. The crude residue was purified by flash column chromatography (SiO₂, EtOAc/Hexanes) to afford the desired product (140 mg, 81% yield). LC-MS calculated for C₃₈H₄₅F₄N₆O₃(M+H)⁺: m/z=709.3; found 709.5.

### Step 2. (Z)-2-(3-(Bicyclo[3.1.1]heptan-3-yloxy)-6-(2-fluoro-2-(4-(pyridazin-4-yl)pyrimidin-2-yl)vinyl)-2-(trifluoromethyl)phenyl)-9-ethyl-2,9-diazaspiro[5.5]undecane

To a mixture of tert-butyl (Z)-2-(3-(cyclohexyloxy)-6-(2-fluoro-2-(6-(pyridazin-4-yl)pyrazin-2-yl)vinyl)-2-(trifluoromethyl)phenyl)-2,9-diazaspiro[5.5]undecane-9-carboxylate (15 mg, 0.025 mmol) in MeOH (2 mL) was added a 4 molar solution of HCl in 1,4-dioxane (2 mL, 8 mmol) and the reaction mixture was stirred at 40° C. for 30 min. After cooling to rt, the solvent was concentrated under reduced pressure, and to the crude residue was added acetonitrile (2 mL), N-ethyl-N-isopropylpropan-2-amine (0.044 mL, 0.25 mmol) and iodoethane (19 mg, 0.13 mmol) and the reaction mixture was stirred at 40° C. for 30 mins. After cooling to rt, the mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₅H₄₁F₄N₆O (M+H)⁺: m/z=637.3; found 637.4.

### Example 55. (Z)-4-(2-(2-(2-(9-(Cyclopropylmethyl)-2,9-diazaspiro[5.5]undecan-2-yl)-4-(2-fluorophenoxy)-3-(trifluoromethyl)phenyl)-1-fluorovinyl)pyrimidin-4-yl)isothiazole

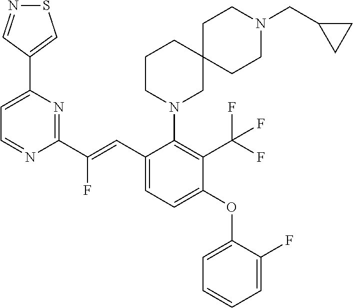 [SMILES: F/C(=C\c1ccc(Oc2ccccc2F)c(C(F)(F)F)c1N1CCCC2(CCN(CC3CC3)CC2)C1)c1nccc(-c2cnsc2)n1]

The title compound was prepared according to the procedures described in Example 46, with (iodomethyl)cyclopropane replacing iodoethane in step 2. LC-MS calculated for C₃₅H₃₅F₅N₅OS (M+H)⁺: m/z=668.2; found 668.3.

### Example 56. (Z)-4-(6-(2-(2-(9-(Cyclopropylmethyl)-2,9-diazaspiro[5.5]undecan-2-yl)-4-(2-fluorophenoxy)-3-(trifluoromethyl)phenyl)-1-fluorovinyl)pyrazin-2-yl)isothiazole

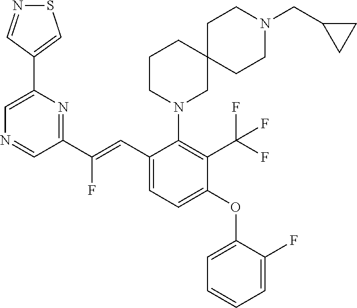 [SMILES: F/C(=C\c1ccc(Oc2ccccc2F)c(C(F)(F)F)c1N1CCCC2(CCN(CC3CC3)CC2)C1)c1cncc(-c2cnsc2)n1]

The title compound was prepared according to the procedures described in Example 46, with 4-(6-chloropyrazin-2-yl)isothiazole (Intermediate 27) replacing 4-(2-iodopyrimidin-4-yl)isothiazole (Intermediate 28) in step 1 and (iodomethyl)cyclopropane replacing iodoethane in step 2. LC-MS calculated for C₃₅H₃₅F₅N₅OS (M+H)⁺: m/z=668.2; found 668.3.

### Example 57. (Z)-9-(Cyclopropylmethyl)-2-(6-(2-(6-(3,3-difluoroazetidin-1-yl)pyrazin-2-yl)-2-fluorovinyl)-3-(2-fluorophenoxy)-2-(trifluoromethyl)phenyl)-2,9-diazaspiro[5.5]undecane

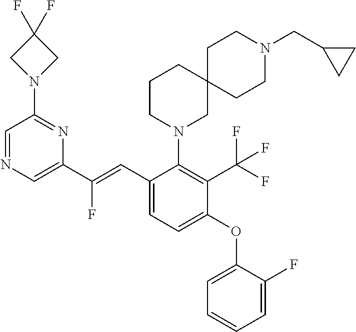 [SMILES: F/C(=C\c1ccc(Oc2ccccc2F)c(C(F)(F)F)c1N1CCCC2(CCN(CC3CC3)CC2)C1)c1cncc(N2CC(F)(F)C2)n1]

This compound was prepared according to the procedures described in Example 39, with 2-bromo-6-(3,3-difluoroazetidin-1-yl)pyrazine (Intermediate 17) replacing 4-(2-bromopyrimidin-4-yl)pyridazine (Intermediate 16) in Step 1. LC-MS calculated for C₃₃H37F₇N₅O (M+H)⁺: m/z=676.3; found 676.3.

### Example 58. (Z)-2-(6-(2-(6-(3,3-Difluoropyrrolidin-1-yl)pyrazin-2-yl)-2-fluorovinyl)-3-(2-fluorophenoxy)-2-(trifluoromethyl)phenyl)-9-ethyl-2,9-diazaspiro[5.5]undecane

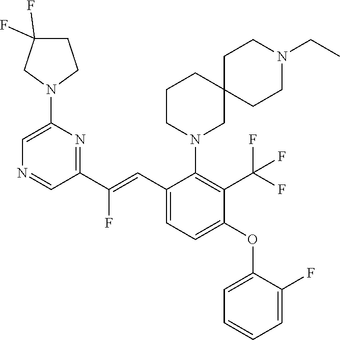 [SMILES: CCN1CCC2(CCCN(c3c(/C=C(\F)c4cncc(N5CCC(F)(F)C5)n4)ccc(Oc4ccccc4F)c3C(F)(F)F)C2)CC1]

This compound was prepared according to the procedures described in Example 19, with tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (Intermediate 24) replacing tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (Intermediate 19) in Step 1 and iodoethane replacing (iodomethyl)cyclopropane in Step 2. LC-MS calculated for C₃₄H₃₇F₇N₅O (M+H)⁺: m/z=664.3; found 664.3.

### Example 59. (Z)-9-(Cyclopropylmethyl)-2-(6-(2-(6-(3,3-difluoropyrrolidin-1-yl)pyrazin-2-yl)-2-fluorovinyl)-3-(2-fluorophenoxy)-2-(trifluoromethyl)phenyl)-2,9-diazaspiro[5.5]undecane

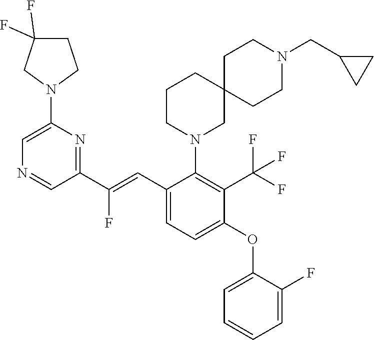 [SMILES: F/C(=C\c1ccc(Oc2ccccc2F)c(C(F)(F)F)c1N1CCCC2(CCN(CC3CC3)CC2)C1)c1cncc(N2CCC(F)(F)C2)n1]

This compound was prepared according to the procedures described in Example 19, with tert-butyl (Z)-2-(6-(2-fluoro-2-(4,4,5,5-tetramethyl-1,3,2-dioxaborolan-2-yl)vinyl)-3-(2-fluorophenoxy)-2-(trifluoromethyl)phenyl)-2,9-diazaspiro[5.5]undecane-9-carboxylate (Intermediate 24) replacing tert-butyl (Z)-2-(6-(2-fluoro-2-(4,4,5,5-tetramethyl-1,3,2-dioxaborolan-2-yl)vinyl)-3-phenoxy-2-(trifluoromethyl)phenyl)-2,9-diazaspiro[5.5]undecane-9-carboxylate (Intermediate 19) in Step 1. LC-MS calculated for C₃₆₃H₃₉F₇N₅O (M+H)⁺: m/z=690.3; found 690.3.

### Example A. In Vitro DGKα and DGKζ Inhibition Assays

The DGKα and DGKζ biochemical reactions were performed using His-tagged human recombinant enzymes (Signal Chem, DGKα, #D21-10BH; DGKζ, #D30-10H)) and DLG (Dilauroyl-sn-glycerol) lipid substrate (Signal Chem, #D430-59). ADP-Glo assay was performed using ADP-Glo™ kinase Assay kit (Promega, #V9104). The reactions were carried out in assay buffer containing 40 mM Tris, pH 7.5, 0.1% CHAPS, 0.1% Prionex, 40 mM NaCl, 5 mM MgCl₂, 1 mM CaCl₂, and 1 mM DTT. DGKα reactions contained 0.1 nM DGKα, 50 μM ATP, and 20 μM DLG. And DGKζ reactions contained 0.4 nM DGKζ, 30 μM ATP, and 20 μM DLG.

For compound inhibition studies, 40 nL test compound in DMSO was added to wells of white polystyrene plates in 384-well (Greiner, #784075) or 1536-well format (Greiner, #782075). Compounds were added with top concentration of 2 mM with 11 point, 3-fold dilution series. Enzyme solution (contains 2× DGK enzyme concentration in 1× assay buffer) was added to the plate in 2 μL/well volume, followed by 2 μL/well of substrate solution (contains 2× concentration of ATP and DLG substrate in 1× assay buffer). Plates were then centrifuged for 1 min at 1200 RPM and sealed or lidded. For 4 μL reaction volume, test compounds were therefore diluted 100× to final top concentration of 20 μM. After 90 minute incubation, reactions were quenched by addition of 2 μL/well Promega ADP-Glo Reagent, followed by centrifugation and lidding. After 60 min incubation, 2 μL/well Promega Kinase Detection Reagent was added, plates centrifuged, and incubated for 30 min. Plates were then read using Luminescence method on BMG PHERAstar FSX plate reader. Percent inhibition was calculated and IC50s were determined using 4-parameter fit in Genedata Screener. Labcyte Echo acoustic dispenser was used for compound addition, and Formulatrix Tempest liquid handler was used for all reagent dispenses.

TABLE A

| Example | DGKα IC₅₀ (nM) | DGKζ IC₅₀ (nM) |
|---|---|---|
| 1 | ++ | ++ |
| 2 | + | + |
| 3 | + | + |
| 4 | + | + |
| 5 | + | ++ |
| 6 | + | + |
| 7 | + | + |
| 8 | + | + |
| 9 | + | + |
| 10 | + | + |
| 11 | ++ | + |
| 12 | + | + |
| 13 | + | + |
| 14 | ++ | ++ |
| 15 | + | + |
| 16 | ++ | + |
| 17 | + | + |
| 18 | + | + |
| 19 | + | + |
| 20 | + | + |
| 21 | + | + |
| 22 | + | + |
| 23 | ++ | + |
| 24 | + | + |
| 25 | + | + |
| 26 | + | + |
| 27 | + | + |
| 28 | + | + |
| 29 | + | + |
| 30 | + | + |
| 31 | + | + |
| 32 | + | + |
| 33 | + | + |
| 34 | + | + |
| 35 | + | + |
| 36 | ++ | + |
| 37 | + | + |
| 38 | + | + |
| 39 | + | + |
| 40 | + | + |
| 41 | + | + |
| 42 | + | + |
| 43 | + | + |
| 44 | + | + |
| 45 | + | + |
| 46 | + | + |
| 47 | + | + |
| 48 | + | + |
| 49 | + | + |
| 50 | + | + |
| 51 | + | + |
| 52 | + | + |
| 53 | + | + |
| 54 | + | + |
| 55 | + | + |
| 56 | + | + |
| 57 | + | + |
| 58 | + | + |
| 59 | + | + |

+ refers to IC₅₀ of ≤20 nM
++ refers to IC₅₀ of >20 nM to ≤200 nM
+++ refers to IC₅₀ of >200 nM to ≤2000 nM
++++ refers to IC₅₀ of >2000 nM

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 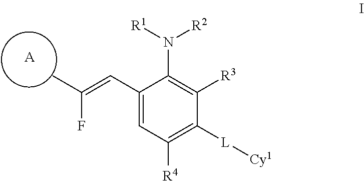
or a pharmaceutically acceptable salt thereof, wherein:
Ring A is: 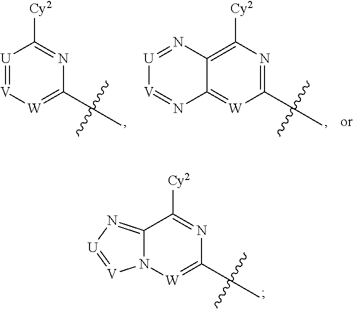
U is CR⁵ or N;
V is CR⁶ or N;
W is CR⁷ or N;
wherein at least one of U, V, or W is not N;
L is a bond, O, or NR¹⁰;
Cy¹ is selected from C₆₋₁₀ aryl, 5-10 membered heteroaryl, 3-10 membered cycloalkyl, and 4-10 membered heterocycloalkyl, wherein the C₆₋₁₀ aryl, 5-10 membered heteroaryl, 3-10 membered cycloalkyl, and 4-10 membered heterocycloalkyl are each optionally substituted with 1, 2, 3, 4, 5, 6, 7, or 8 independently selected R¹¹ substituents;
each R¹¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a111}, SR^{a111}, NHOR^{a111}, C(O)R^{b111}, C(O)NR^{c111}R^{d111}, C(O)NR^{c111}(OR^{a111}), C(O)OR^{a111}, OC(O)R^{b111}, OC(O)NR^{c111}R^{d111}, NR^{c111}R^{d111}, NR^{c111}R^{c111}R^{d111}, NR^{c111}C(O)R^{b111}, NR^{c111}C(O)OR^{a111}, NR^{c111}C(O)NR^{c111}R^{d111}, C(═NR^{e111})R^{b111}, C(═NR^{e111})NR^{c111}R^{d111}, NR^{c111}C(═NR^{e111})NR^{c111}R^{d111}, NR^{e111}C(═NR^{e111})R^{b111}, NR^{c111}S(O)R^{b111}, NR^{c111}S(O)NR^{c111}R^{d111}, NR^{c111}S(O)₂R^{b111}, NR^{c111}S(O)(═NR^{e111})R^{b111}, NR^{c111}S(O)₂NR^{c111}R^{d111}, S(O)R^{b111}, S(O)NR^{c111}R^{d111}, S(O)₂R^{b111}, S(O)₂NR^{c111}R^{d111}, OS(O)(═NR^{e111})R^{b111}, and OS(O)₂R^{b111}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl- of R¹¹ are each optionally substituted with 1, 2, 3, 4, 5, 6, 7, or 8 independently selected R^{11A} substituents;
each R^{a111}, R^{c111}, and R^{d111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a111}, R^{c111} and R^{d11} are each optionally substituted with 1, 2, 3, 4, 5, 6, 7, or 8 independently selected R^{11A} substituents;
or, any R^{c111} and R^{d111} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{11A} substituents;
each R^{b111} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b111} are each optionally substituted with 1, 2, 3, 4, 5, 6, 7, or 8 independently selected R^{11A} substituents;
each R^{e111} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{11A} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a112}, C(O)NR^{c112}R^{d112}, C(O)OR^{a112}, NR^{c112}R^{d112}, S(O)NR^{c112}R^{d112}, S(O)₂R^{b112}, S(O)₂NR^{c112}R^{a112}, and OS(O)₂R^{b112}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{11A} are each optionally substituted with 1, 2, 3, or 4 independently selected R^{M} substituents;
each R^{a112}, R^{c112}, and R^{d112} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a112}, R^{c112} and R^{d112} are each optionally substituted with 1, 2, 3, or 4 independently selected R^{M} substituents;
or, any R^{c112} and R^{d112} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b112} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b112} are each optionally substituted with 1, 2, 3, or 4 independently selected R^{M} substituents;
Cy² is 4-pyridazinyl, 5 membered heteroaryl, or 4-8 membered heterocycloalkyl, wherein the 4-pyridazinyl, 5 membered heteroaryl, and 4-8 membered heterocycloalkyl are each optionally substituted with 1, 2, 3, or 4 independently selected R¹² substituents;
R¹² is selected from halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OR^{a121}, CN, C(O)OH, C(O)NHR^{a121}, and NR^{a121}R^{a121}, wherein the C₁₋₆ alkyl is optionally substituted with OH, CN, and NH₂;
each R^{a121} is independently selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R¹ is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, C(═NR^{e1})R^{b1}, C(═NR^{e1})NR^{c1}R^{d1}, C(═NOR^{a1})R^{b1}, C(═NOR^{a1})OR^{a1}, S(O)R^{b1}, S(O)NR^{c1}R^{d1}, S(O)₂R^{b1}, S(O)₂NR^{c1}R^{d1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹ are each optionally substituted with 1, 2, 3, 4, 5, 6, 7, or 8 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a1}, R^{c1} and R^{d1} are each optionally substituted with 1, 2, 3, 4, 5, 6, 7, or 8 independently selected R^{1A} substituents;
or, any R^{c1} and R^{d1} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{1A} substituents;
each R^{b1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b1} are each optionally substituted with 1, 2, 3, 4, 5, 6, 7, or 8 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11} NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, 4, 5, 6, 7, or 8 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a11}, R^{c11} and R^{d11} are each optionally substituted with 1, 2, 3, 4, 5, 6, 7, or 8 independently selected R^{1B} substituents;
or, any R^{c11} and R^{d11} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{1B} substituents;
each R^{b11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b11} are each optionally substituted with 1, 2, 3, 4, 5, 6, 7, or 8 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1B} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a12}, C(O)NR^{c12}R^{d12}, C(O)OR^{a12}, NR^{c12}R^{d12}, S(O)NR^{c12}R^{d12}, S(O)₂R^{b12}, S(O)₂NR^{c12}R^{d12}, and OS(O)₂R^{b12}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{1B} are each optionally substituted with 1, 2, 3, or 4 independently selected R^{M} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a12}, R^{c12} and R^{d12} are each optionally substituted with 1, 2, 3, or 4 independently selected R^{M} substituents;
or, any R^{c12} and R^{d12} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b12} are each optionally substituted with 1, 2, 3, or 4 independently selected R^{M} substituents;
R² is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, C(═NR^{e2})R^{b2}, C(═NR^{e2})NR^{c2}R^{d2}, C(═NOR^{a2})R^{b2}, C(═NOR^{a2})OR^{a2}, S(O)R^{b2}, S(O)NR^{c2}R^{d2}, S(O)₂R^{b2}, and S(O)₂NR^{c2}R^{d2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR²¹)R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2B} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a22}, C(O)NR^{c22}R^{d22}, C(O)OR^{a22}, NR^{c22}R^{d22}, S(O)NR^{c22}R^{d22}, S(O)₂R^{b22}, S(O)₂NR^{c22}R^{d22}, and OS(O)₂R^{b22}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{2B} are each optionally substituted with 1, 2, 3, or 4 independently selected R^{M} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a22}, R^{c22} and R^{d22} are each optionally substituted with 1, 2, 3, or 4 independently selected R^{M} substituents;
or, any R^{c22} and R^{d22} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b22} are each optionally substituted with 1, 2, 3, or 4 independently selected R^{M} substituents;
or, R¹ and R², together with the N atom to which they are attached, form a 4-12 membered heterocycloalkyl, wherein the 4-12 membered heterocycloalkyl is optionally substituted with 1, 2, 3, 4, 5, 6, 7, or 8 independently selected R¹³ substituents;
each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a131}, SR^{a131}, NHOR^{a131}, C(O)R^{b131}, C(O)NR^{c131}R^{d131}, C(O)NR^{c131}(OR^{a131}), C(O)OR^{a131}, OC(O)R^{b131}, OC(O)NR^{c131}R^{d131}, NR^{c131}R^{d131}, NR^{c131}NR^{c131}R^{d131}, NR^{c131}C(O)R^{b131}, NR^{c131}C(O)OR^{a131}, NR^{c131}C(O)NR^{c131}R^{d131}, C(═NR^{e131})R^{b131}, C(═NR^{e131})NR^{c131}R^{d131}, C(═NOR^{a131})R^{b131}, C(═NOR^{a131})OR^{a131}, NR^{c131}C(═NR^{c131})NR^{c131}R^{d131}, NR^{c131}C(═NR^{e131})R^{b131}, NR^{c131}S(O)R^{b131}, NR^{c131}S(O)NR^{c131}R^{d131}, NR^{c131}S(O)₂R^{b131}, NR^{c131}S(O)(═NR^{e131})R^{b131}, NR^{c131}S(O)₂NR^{c131}R^{d131}, S(O)R^{b131}, S(O)NR^{c131}R^{d131}, S(O)₂R^{b131}, S(O)₂NR^{c131}R^{d131}, OS(O)(═NR^{e131})R^{b131}, and OS(O)₂R^{b131}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1, 2, 3, 4, 5, 6, 7, or 8 independently selected R^{13A} substituents;
each R^{a131}, R^{c131}, and R^{d131} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a131}, R^{c131} and R^{d131} are each optionally substituted with 1, 2, 3, 4, 5, 6, 7, or 8 independently selected R^{13A} substituents;
or, any R^{c131} and R^{d131} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{13A} substituents;
each R^{b131} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b1}31 are each optionally substituted with 1, 2, 3, 4, 5, 6, 7, or 8 independently selected R^{13A} substituents;
each R^{e131} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{13A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a132}, SR^{a132}, NHOR^{a132}, C(O)R^{b132}, C(O)NR^{c132}R^{d132}, C(O)NR^{c132}(OR^{a132}), C(O)OR^{a132}, OC(O)R^{b132}, OC(O)NR^{c132}R^{d132} NR^{c132}R^{d132}, NR^{c132}NR^{c132}R^{d132}, NR^{c132}C(O)R^{b132}, NR^{c132}C(O)OR^{a132}, NR^{c132}C(O)NR^{c132}R^{d132}, C(═NR^{e132})R^{b132}, C(═NR^{e132})R^{c132}R^{d132}, NR^{c132}C(═NR^{e132})NR^{c132}R^{d132}, NR^{c132}C(═NR^{e132})R^{b132}, NR^{c132}S(O)R^{b132}, NR^{c132}S(O)NR^{c132}R^{d132}, NR^{c132}S(O)₂R^{b132}, NR^{c132}S(O)(═NR^{e132})R^{b132}, NR^{c132}S(O)₂NR^{c132}R^{d132}, S(O)R^{b132}, S(O)NR^{c132}R^{d132}, S(O)₂R^{b132}, S(O)₂NR^{c132}R^{d132}, OS(O)(═NR^{e132})R^{b132}, and OS(O)₂R^{b132}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{13A} are each optionally substituted with 1, 2, 3, 4, 5, 6, 7, or 8 independently selected R^{13B} substituents;
each R^{a132}, R^{c132}, and R^{d132} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a132}, R^{c132} and R^{d132} are each optionally substituted with 1, 2, 3, 4, 5, 6, 7, or 8 independently selected R^{13B} substituents;
or, any R^{c132} and R^{d132} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{13B} substituents;
each R^{b132} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b132} are each optionally substituted with 1, 2, 3, 4, 5, 6, 7, or 8 independently selected R^{13B} substituents;
each R^{e132} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{13B} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a133}, C(O)NR^{c133}R^{d133}, C(O)OR^{a133}, NR^{c133}R^{d133}, S(O)NR^{c133}R^{d133}, S(O)₂R^{b133}, S(O)₂NR^{c133}R^{d133}, and OS(O)₂R^{b133}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{13B} are each optionally substituted with 1, 2, 3, or 4 independently selected R^{M} substituents;
each R^{a133}, R^{c133}, and R^{d133} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a133}, R^{c133} and R^{d133} are each optionally substituted with 1, 2, 3, or 4 independently selected R^{M} substituents;
or, any R^{c133} and R^{d133} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b133} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b133} are each optionally substituted with 1, 2, 3, or 4 independently selected R^{M} substituents;
R³ is selected from H, halo, C₁₋₆ alkyl and C₁₋₆ haloalkyl;
R⁴ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₄ cycloalkyl, OH, CN, C(O)OH, C(O)NH₂, and NH₂; wherein the C₁₋₆ alkyl is optionally substituted with OH, CN, and NH₂;
R⁵ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a51}, and NHR^{a52}, wherein the C₁₋₆ alkyl is optionally substituted with OH, CN, and NH₂;
each R^{a51} is independently selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
each R^{a52} is independently selected from C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R⁶ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, and C(O)NH₂; wherein the C₁₋₆ alkyl is optionally substituted with OH, CN, and NH₂;
R⁷ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a71}, and NHR^{a72}, wherein the C₁₋₆ alkyl is optionally substituted with OH, CN, and NH₂;
each R^{a71} is independently selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
each R^{a72} is independently selected from C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R¹⁰ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NH₂, and NH₂; wherein the C₁₋₆ alkyl, C₂₋₆ alkenyl, and C₂₋₆ alkynyl of R¹⁰ are each optionally substituted with 1, 2, 3, or 4 independently selected R^{M} substituents; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-.

2. The compound of claim 1, or a pharmaceutically acceptable salt thereof, wherein Ring A is

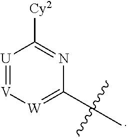

3. The compound of claim 1, or a pharmaceutically acceptable salt thereof, wherein Ring A is

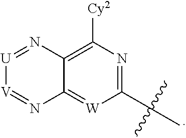

4. The compound of claim 1, or a pharmaceutically acceptable salt thereof, wherein Ring A is

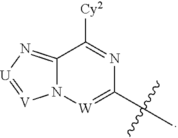

5. The compound of claim 1, or a pharmaceutically acceptable salt thereof, wherein U is CRS.

6. The compound of claim 1, or a pharmaceutically acceptable salt thereof, wherein R⁵ is selected from H and C₁₋₆ alkyl.

7. The compound of claim 1, or a pharmaceutically acceptable salt thereof, wherein U is CH.

8. The compound of claim 1, or a pharmaceutically acceptable salt thereof, wherein U is N.

9. The compound of claim 1, or a pharmaceutically acceptable salt thereof, wherein V is CR⁶.

10. The compound of claim 1, or a pharmaceutically acceptable salt thereof, wherein R⁶ is selected from H and C₁₋₆ alkyl.

11. The compound of claim 1, or a pharmaceutically acceptable salt thereof, wherein V is CH.

12. The compound of claim 1, or a pharmaceutically acceptable salt thereof, wherein V is N.

13. The compound of claim 1, or a pharmaceutically acceptable salt thereof, wherein W is CR⁷.

14. The compound of claim 1, or a pharmaceutically acceptable salt thereof, wherein R⁷ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, and CN.

15. The compound of claim 1, or a pharmaceutically acceptable salt thereof, wherein R⁷ is selected from H and CN.

16. The compound of claim 1, or a pharmaceutically acceptable salt thereof, wherein W is N.

17. The compound of claim 1, or a pharmaceutically acceptable salt thereof, wherein Cy² is 4-pyridazinyl, 5 membered heteroaryl, or 4-6 membered heterocycloalkyl, wherein the 4-pyridazinyl, 5 membered heteroaryl, and 4-6 membered heterocycloalkyl are each optionally substituted with 1, 2, 3, or 4 independently selected R¹² substituents.

18. The compound of claim 1, or a pharmaceutically acceptable salt thereof, wherein Cy² is 4-pyridazinyl, isothiazolyl, azetidinyl, or pyrrolidinyl, wherein the 4-pyridazinyl, isothiazolyl, azetidinyl, or pyrrolidinyl are each optionally substituted with 1, 2, 3, or 4 independently selected R¹² substituents.

19. The compound of claim 1, or a pharmaceutically acceptable salt thereof, wherein each R¹² is independently selected from halo, C₁₋₆ alkyl, C₁₋₆ alkoxy, C₁₋₆ haloalkyl, C₁₋₆ haloalkoxy, C₂₋₆ alkenyl, and C₂₋₆ alkynyl.

20. The compound of claim 1, or a pharmaceutically acceptable salt thereof, wherein each R¹² is an independently selected halo.

21. The compound of claim 1, or a pharmaceutically acceptable salt thereof, wherein each R¹² is fluoro.

22. The compound of claim 1, or a pharmaceutically acceptable salt thereof, wherein Cy² is 4-pyridizinyl, isothiazolyl, difluoroazetidinyl, or difluoropyrrolidinyl.

23. The compound of claim 1, or a pharmaceutically acceptable salt thereof, wherein R¹ is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R¹ are each optionally substituted with 1, 2, 3, or 4 independently selected R^{1A} substituents.

24. The compound of claim 1, or a pharmaceutically acceptable salt thereof, wherein R¹ is C₁₋₆ alkyl, which is optionally substituted with 1 or 2 independently selected R^{1A} substituents.

25. The compound of claim 1, or a pharmaceutically acceptable salt thereof, wherein each R^{1A} is independently selected from halo, C₁₋₆ alkyl, C₁₋₆ haloalkyl, C₂₋₆ alkenyl, C₂₋₆ alkynyl, and NR^{c11}R^{d11}; and
each R^{c11} and R^{d11} is independently selected from H and C₁₋₆ alkyl.

26. The compound of claim 1, or a pharmaceutically acceptable salt thereof, wherein each R^{1A} is NR^{c11}R^{d11}; and
each R^{c11} and R^{d11} is independently selected from H and C₁₋₆ alkyl.

27. The compound of claim 1, or a pharmaceutically acceptable salt thereof, wherein R¹ is methyl.

28. The compound of claim 1, or a pharmaceutically acceptable salt thereof, wherein R² is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{2A} substituents.

29. The compound of claim 1, or a pharmaceutically acceptable salt thereof, wherein R² is C₁₋₆ alkyl, which is optionally substituted with 1 or 2 independently selected R^{2A} substituents.

30. The compound of claim 1, or a pharmaceutically acceptable salt thereof, wherein each R^{2A} is independently selected from halo, C₁₋₆ alkyl, C₁₋₆ haloalkyl, C₂₋₆ alkenyl, C₂₋₆ alkynyl, and NR^{c21}R^{d21}; and
each R^{c21} and R^{d21} is independently selected from H and C₁₋₆ alkyl.

31. The compound of claim 1, or a pharmaceutically acceptable salt thereof, wherein each R^{2A} is NR^{c21}R^{d21}; and
each R^{c21} and R^{d21} is independently selected from H and C₁₋₆ alkyl.

32. The compound of claim 1, or a pharmaceutically acceptable salt thereof, wherein R² is aminopropyl.

33. The compound of claim 1, or a pharmaceutically acceptable salt thereof, wherein R¹ and R², together with the N atom to which they are attached, form a 4-12 membered heterocycloalkyl, wherein the 4-12 membered heterocycloalkyl is optionally substituted with 1, 2, 3, or 4 independently selected R¹³ substituents.

34. The compound of claim 1, or a pharmaceutically acceptable salt thereof, wherein R¹ and R², together with the N atom to which they are attached, form a 4-7 membered monocyclic heterocycloalkyl or an 8-12 membered spirocyclic heterocycloalkyl, wherein the 4-7 membered monocyclic heterocycloalkyl and 8-12 membered spirocyclic heterocycloalkyl are each optionally substituted with 1, 2, 3, or 4 independently selected R¹³ substituents.

35. The compound of claim 1, or a pharmaceutically acceptable salt thereof, wherein R¹ and R², together with the N atom to which they are attached, form a pyrrolidinyl, piperidinyl, 2,9-diazaspiro[5.5]undecanyl, or 1-oxa-4,9-diazaspiro[5.5]undecanyl, wherein the pyrrolidinyl, piperidinyl, 2,9-diazaspiro[5.5]undecanyl, and 1-oxa-4,9-diazaspiro[5.5]undecanyl are each optionally substituted with 1, 2, 3, or 4 independently selected R¹³ substituents.

36. The compound of claim 1, or a pharmaceutically acceptable salt thereof, wherein each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1, 2, 3, or 4 independently selected R^{13A} substituents.

37. The compound of claim 1, or a pharmaceutically acceptable salt thereof, wherein each R¹³ is independently selected from halo, oxo, C₁₋₆ alkyl, C₁₋₆ haloalkyl, C₂₋₆ alkenyl, C₂₋₆ alkynyl, C₃₋₇ cycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1, 2, 3, or 4 independently selected R^{13A} substituents.

38. The compound of claim 1, or a pharmaceutically acceptable salt thereof, wherein each R¹³ is independently selected from C₁₋₆ alkyl, C₃₋₇ cycloalkyl, and C₃₋₇ cycloalkyl-C₁₋₆ alkyl-, wherein the C₁₋₆ alkyl, C₃₋₇ cycloalkyl, and C₃₋₇ cycloalkyl-C₁₋₆ alkyl- of R¹³ are each optionally substituted with 1 or 2 independently selected R^{13A} substituents.

39. The compound of claim 1, or a pharmaceutically acceptable salt thereof, wherein each R^{13A} is independently selected from halo, C₁₋₆ alkyl, C₁₋₆ haloalkyl, C₂₋₆ alkenyl, C₂₋₆ alkynyl, OR^{a132}, and NR^{e132}R^{d132};
each R^{a132}, R^{c132}, and R^{d132} is independently selected from H and C₁₋₆ alkyl.

40. The compound of claim 1, or a pharmaceutically acceptable salt thereof, wherein each R^{13A} is independently selected amino, hydroxy, and methoxy.

41. The compound of claim 1, or a pharmaceutically acceptable salt thereof, wherein each R¹³ is independently selected from ethyl, cyclobutyl, cyclopropylmethyl, aminomethyl, hydroxyethyl, and methoxyethyl.

42. The compound of claim 1, or a pharmaceutically acceptable salt thereof, wherein R³ is selected from halo and C₁₋₆ haloalkyl.

43. The compound of claim 1, or a pharmaceutically acceptable salt thereof, wherein R³ is selected from chloro and trifluoromethyl.

44. The compound of claim 1, or a pharmaceutically acceptable salt thereof, wherein R⁴ is selected from H, halo, C₁₋₆ alkyl, C₁₋₆ alkoxy, C₁₋₆ haloalkyl, C₁₋₆ haloalkoxy, C₂₋₆ alkenyl, and C₂₋₆ alkynyl.

45. The compound of claim 1, or a pharmaceutically acceptable salt thereof, wherein R⁴ is H.

46. The compound of claim 1, or a pharmaceutically acceptable salt thereof, wherein L is O.

47. The compound of claim 1, or a pharmaceutically acceptable salt thereof, wherein Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl are each optionally substituted with 1, 2, 3, or 4 independently selected R¹¹ substituents.

48. The compound of claim 1, or a pharmaceutically acceptable salt thereof, wherein Cy¹ is selected from phenyl, 5-6 membered heteroaryl, and C₃₋₇ cycloalkyl, wherein the phenyl, 5-6 membered heteroaryl, and C₃₋₇ cycloalkyl are each optionally substituted with 1, 2, 3, or 4 independently selected R¹¹ substituents.

49. The compound of claim 1, or a pharmaceutically acceptable salt thereof, wherein Cy¹ is selected from phenyl, pentadeuterophenyl, pyridinyl, cyclopropyl, cyclobutyl, cyclopentyl, cyclohexyl, and bicyclo[3.1.1]heptanyl, wherein the phenyl, pyridinyl, cyclopropyl, cyclobutyl, cyclopentyl, cyclohexyl, and bicyclo[3.1.1]heptanyl are each optionally substituted with 1, 2, 3, or 4 independently selected R¹¹ substituents.

50. The compound of claim 1, or a pharmaceutically acceptable salt thereof, wherein each R¹¹ is independently selected from halo, C₁₋₆ alkyl, C₁₋₆ haloalkyl, C₂₋₆ alkenyl, and OR^{a111}; and
each R^{a111} is independently selected from H and C₁₋₆ alkyl.

51. The compound of claim 1, or a pharmaceutically acceptable salt thereof, wherein each R¹¹ is independently selected from halo and OR^{a111}; and
each R^{a111} is independently selected from H and C₁₋₆ alkyl.

52. The compound of claim 1, or a pharmaceutically acceptable salt thereof, wherein each R¹¹ is independently selected from fluoro and methoxy.

53. The compound of claim 1, or a pharmaceutically acceptable salt thereof, wherein Cy¹ is selected from cyclopropyl, cyclobutyl, cyclopentyl, cyclohexyl, bicyclo[3.1.1]heptanyl, fluoropyridinyl, phenyl, pentadeuterophenyl, fluorophenyl, difluorophenyl, and (fluoro)(methoxy)phenyl.

54. The compound of claim 1, or a pharmaceutically acceptable salt thereof, wherein:
Ring A is: 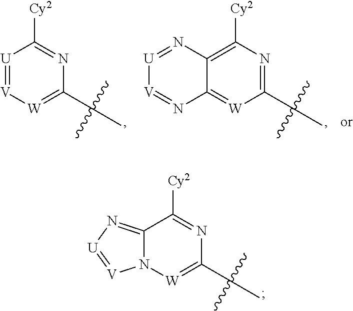
U is CR⁵ or N;
V is CR⁶ or N;
W is CR⁷ or N;
wherein at least one of U, V, or W is not N;
L is a bond, O, or NR¹⁰;
Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl are each optionally substituted with 1, 2, 3, or 4 independently selected R¹¹ substituents;
each R¹¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a111}, SR^{a111}, NHOR^{a111}, C(O)R^{b111}, C(O)NR^{c111}R^{d111}, C(O)NR^{c111}(OR^{a111}), C(O)OR^{a111}, OC(O)R^{b111}, OC(O)NR^{c111}R^{d111}, NR^{c111}R^{d111}, NR^{c111}NR^{c111}R^{d111}, NR^{c111}C(O)R^{b111}, NR^{c111}C(O)OR^{a111}, NR^{c111}C(O)NR^{c111}R^{d111}, C(═NR^{e111})R^{b111}, C(═NR^{e111})NR^{c111}R^{d111}, NR^{c111}C(═NR^{e111})NR^{c111}R^{d111}, NR^{c111}C(═NR^{e111})R^{d111}, NR^{c111}S(O)R^{b111}, NR^{c111}(O)NR^{c111}R^{d111}, NR^{c111}S(O)₂R^{b111}, NR^{c111}S(O)(═NR^{e111})R^{b111}, NR^{c111}S(O)₂NR^{c111}R^{d111}, S(O)R^{b111}, S(O)NR^{c111}R^{d111}, S(O)₂R^{b111}, S(O)₂NR^{c111}R^{d111}, OS(O)(═NR^{e111})R^{b111}, and OS(O)₂R^{b111};
each R^{a111}, R^{c111}, and R^{d111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c111} and R^{d111} attached to the same N atom, together with the N atom to which they are attached, form a 5-10 membered heteroaryl or a 4-10 membered heterocycloalkyl group;
each R^{b111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e111} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
Cy² is 4-pyridazinyl, 5 membered heteroaryl, or 4-8 membered heterocycloalkyl, wherein the 4-pyridazinyl, 5 membered heteroaryl, and 4-8 membered heterocycloalkyl are each optionally substituted with 1, 2, 3, or 4 independently selected R¹² substituents;
R¹² is selected from halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OR^{a121}, CN, C(O)OH, C(O)NHR^{a121}, and NR^{a121}R^{a121}, wherein the C₁₋₆ alkyl is optionally substituted with OH, CN, and NH₂;
each R^{a121} is independently selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R¹ is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R¹ are each optionally substituted with 1, 2, 3, or 4 independently selected R^{1A} substituents;
each R^{1A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}(═NR^{e11})NR^{c11}R^{d11}, NR^{c11}C(═NR^{e11})R^{b11}, NR^{c11}S(O)R^{b11}, NR^{c11}(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11};
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c11} and R^{d11} attached to the same N atom, together with the N atom to which they are attached, form a 5-10 membered heteroaryl or a 4-10 membered heterocycloalkyl group;
each R^{b11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
R² is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}NR^{c21}C(O)NR^{c21}R^{d21}, C(═NR²¹)R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21};
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c21} and R^{d21} attached to the same N atom, together with the N atom to which they are attached, form a 5-10 membered heteroaryl or a 4-10 membered heterocycloalkyl group;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or R¹ and R², together with the N atom to which they are attached, form a 4-12 membered heterocycloalkyl, wherein the 4-12 membered heterocycloalkyl is optionally substituted with 1, 2, 3, or 4 independently selected R¹³ substituents;
each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1, 2, 3, or 4 independently selected R^{13A} substituents;
each R^{13A} is independently selected from halo, C₁₋₆ alkyl, C₁₋₆ haloalkyl, C₂₋₆ alkenyl, C₂₋₆ alkynyl, OR^{a132}, and NR^{c132}R^{d132};
each R^{a132}, R^{c132}, and R^{d132} is independently selected from H and C₁₋₆ alkyl;
R³ is selected from H, halo, C₁₋₆ alkyl and C₁₋₆ haloalkyl;
R⁴ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₄ cycloalkyl, OH, CN, C(O)OH, C(O)NH₂, and NH₂; wherein the C₁₋₆ alkyl is optionally substituted with OH, CN, and NH₂;
R⁵ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a51}, and NHR^{a52}, wherein the C₁₋₆ alkyl is optionally substituted with OH, CN, and NH₂;
each R^{a51} is independently selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
each R^{a52} is independently selected from C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R⁶ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, and C(O)NH₂; wherein the C₁₋₆ alkyl is optionally substituted with OH, CN, and NH₂; and
R⁷ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a71}, and NHR^{a72}, wherein the C₁₋₆ alkyl is optionally substituted with OH, CN, and NH₂;
R¹⁰ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NH₂, and NH₂; wherein the C₁₋₆ alkyl, C₂₋₆ alkenyl, and C₂₋₆ alkynyl of R¹⁰ are each optionally substituted with 1, 2, 3, or 4 independently selected R^{M} substituents; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl.

55. The compound of claim 1, or a pharmaceutically acceptable salt thereof, wherein:
Ring A is: 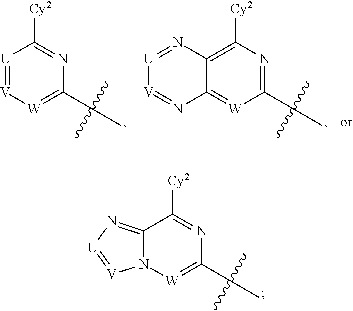
U is CR⁵ or N;
V is CR⁶ or N;
W is CR⁷ or N;
wherein at least one of U, V, or W is not N;
L is a bond, O, or NR¹⁰;
Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl are each optionally substituted with 1, 2, 3, or 4 independently selected R¹¹ substituents;
R¹¹ is independently selected from halo, C₁₋₆ alkyl, C₁₋₆ haloalkyl, C₂₋₆ alkenyl, and OR^{a111};
each R^{a111} is independently selected from H and C₁₋₆ alkyl;
Cy² is 4-pyridazinyl, 5 membered heteroaryl, or 4-6 membered heterocycloalkyl, wherein the 4-pyridazinyl, 5 membered heteroaryl, and 4-6 membered heterocycloalkyl are each optionally substituted with 1, 2, 3, or 4 independently selected R¹² substituents;
R¹² is independently selected from halo, C₁₋₆ alkyl, C₁₋₆ alkoxy, C₁₋₆ haloalkyl, C₁₋₆ haloalkoxy, C₂₋₆ alkenyl, and C₂₋₆ alkynyl;
R¹ is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R¹ are each optionally substituted with 1, 2, 3, or 4 independently selected R^{1A} substituents;
each R^{1A} is independently selected from halo, C₁₋₆ alkyl, C₁₋₆ haloalkyl, C₂₋₆ alkenyl, C₂₋₆ alkynyl, and NR^{c11}R^{d11};
each R^{c11} and R^{d11} is independently selected from H and C₁₋₆ alkyl;
R² is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, and NR^{c21}R^{d21};
each R^{c21} and R^{d21} is independently selected from H and C₁₋₆ alkyl;
or R¹ and R², together with the N atom to which they are attached, form a 4-12 membered heterocycloalkyl, wherein the 4-12 membered heterocycloalkyl is optionally substituted with 1, 2, 3, or 4 independently selected R¹³ substituents;
each R¹³ is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R¹³ are each optionally substituted with 1, 2, 3, or 4 independently selected R^{13A} substituents;
each R^{13A} is independently selected from halo, C₁₋₆ alkyl, C₁₋₆ haloalkyl, C₂₋₆ alkenyl, C₂₋₆ alkynyl, OR^{a132}, and NR^{c132}R^{d132};
each R^{a132}, R^{c132}, and R^{d132} is independently selected from H and C₁₋₆ alkyl;
R³ is selected from halo and C₁₋₆ haloalkyl;
R⁴ is selected from H, halo, C₁₋₆ alkyl, C₁₋₆ alkoxy, C₁₋₆ haloalkyl, C₁₋₆ haloalkoxy, C₂₋₆ alkenyl, and C₂₋₆ alkynyl;
R⁵ is selected from H and C₁₋₆ alkyl;
R⁶ is selected from H and C₁₋₆ alkyl;
R⁷ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, and CN;
R¹⁰ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NH₂, and NH₂; wherein the C₁₋₆ alkyl, C₂₋₆ alkenyl, and C₂₋₆ alkynyl of R¹⁰ are each optionally substituted with 1, 2, 3, or 4 independently selected R^{M} substituents; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl.

56. The compound of claim 1, wherein the compound of Formula I is a compound of Formula II: 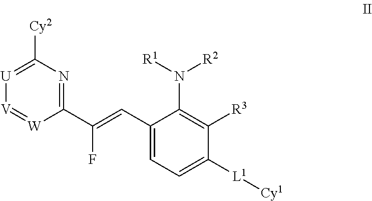
or a pharmaceutically acceptable salt thereof.

57. The compound of claim 1, wherein the compound of Formula I is a compound of Formula III: 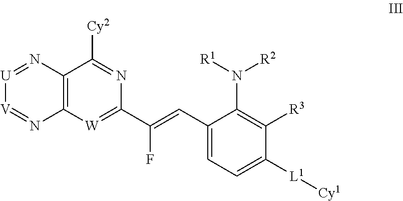
or a pharmaceutically acceptable salt thereof.

58. The compound of claim 1, wherein the compound of Formula I is a compound of Formula IV: 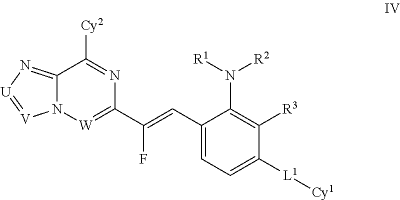
or a pharmaceutically acceptable salt thereof.

59. The compound of claim 1, which is selected from:
(S,Z)-(1-(6-(2-(5-(3,3-difluoroazetidin-1-yl)pyrido[3,4-b]pyrazin-7-yl)-2-fluorovinyl)-3-(2-fluorophenoxy)-2-(trifluoromethyl)phenyl)piperidin-3-yl)methanamine;
(S,Z)-(1-(6-(2-(8-(3,3-difluoroazetidin-1-yl)imidazo[1,2-a]pyrazin-6-yl)-2-fluorovinyl)-3-(2-fluorophenoxy)-2-(trifluoromethyl)phenyl)piperidin-3-yl)methanamine;
(S,Z)-3-(2-(2-(3-(aminomethyl)piperidin-1-yl)-4-(2-fluorophenoxy)-3-(trifluoromethyl)phenyl)-1-fluorovinyl)-5-(3,3-difluoroazetidin-1-yl)pyrazine-2-carbonitrile;
(S,Z)-(1-(6-(2-fluoro-2-(6-(isothiazol-5-yl)pyrazin-2-yl)vinyl)-3-(2-fluorophenoxy)-2-(trifluoromethyl)phenyl)piperidin-3-yl)methanamine;
(Z)-N1-(6-(2-fluoro-2-(6-(pyridazin-4-yl)pyridin-2-yl)vinyl)-3-phenoxy-2-(trifluoromethyl)phenyl)-N1-methylpropane-1,3-diamine;
(S,Z)-(1-(2-chloro-6-(2-fluoro-2-(4-(pyridazin-4-yl)pyrimidin-2-yl)vinyl)-3-(4-fluoro-2-methoxyphenoxy)phenyl)piperidin-3-yl)methanamine;
(S,Z)-(1-(2-chloro-6-(2-fluoro-2-(2-(pyridazin-4-yl)pyrimidin-4-yl)vinyl)-3-(4-fluoro-2-methoxyphenoxy)phenyl)piperidin-3-yl)methanamine;
(S,Z)-(1-(2-chloro-6-(2-(5-(3,3-difluoropyrrolidin-1-yl)-1,2,4-triazin-3-yl)-2-fluorovinyl)-3-(4-fluoro-2-methoxyphenoxy)phenyl)piperidin-3-yl)methanamine;
(Z)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane;
(Z)-9-(cyclopropylmethyl)-4-(6-(2-fluoro-2-(6-(pyridazin-4-yl)pyrazin-2-yl)vinyl)-3-phenoxy-2-(trifluoromethyl)phenyl)-1-oxa-4,9-diazaspiro[5.5]undecane;
(Z)-4-(6-(2-fluoro-2-(6-(pyridazin-4-yl)pyrazin-2-yl)vinyl)-3-phenoxy-2-(trifluoromethyl)phenyl)-9-(2-methoxyethyl)-1-oxa-4,9-diazaspiro[5.5]undecane;
(R,Z)-(1-(2-chloro-6-(2-fluoro-2-(6-(pyridazin-4-yl)pyrazin-2-yl)vinyl)-3-phenoxyphenyl)pyrrolidin-2-yl)methanamine;
(R,Z)-(1-(2-chloro-6-(2-fluoro-2-(4-(pyridazin-4-yl)pyrimidin-2-yl)vinyl)-3-phenoxyphenyl)pyrrolidin-2-yl)methanamine; and
(Z)-4-(3-cyclopropoxy-6-(2-fluoro-2-(6-(pyridazin-4-yl)pyrazin-2-yl)vinyl)-2-(trifluoromethyl)phenyl)-9-ethyl-1-oxa-4,9-diazaspiro[5.5]undecane;
or a pharmaceutically acceptable salt thereof.

60. The compound of claim 1, which is selected from:
(Z)-9-(cyclopropylmethyl)-2-(6-(2-(6-(3,3-difluoroazetidin-1-yl)pyrazin-2-yl)-2-fluorovinyl)-3-phenoxy-2-(trifluoromethyl)phenyl)-2,9-diazaspiro[5.5]undecane;
(Z)-2-(6-(2-(6-(3,3-difluoroazetidin-1-yl)pyrazin-2-yl)-2-fluorovinyl)-3-phenoxy-2-(trifluoromethyl)phenyl)-9-ethyl-2,9-diazaspiro[5.5]undecane;
(Z)-9-(cyclopropylmethyl)-2-(6-(2-(6-(3,3-difluoroazetidin-1-yl)pyrazin-2-yl)-2-fluorovinyl)-3-(phenoxy-ds)-2-(trifluoromethyl)phenyl)-2,9-diazaspiro[5.5]undecane;
(Z)-2-(6-(2-(6-(3,3-difluoroazetidin-1-yl)pyrazin-2-yl)-2-fluorovinyl)-3-(phenoxy-ds)-2-(trifluoromethyl)phenyl)-9-ethyl-2,9-diazaspiro[5.5]undecane;
(Z)-9-(cyclopropylmethyl)-2-(6-(2-(6-(3,3-difluoropyrrolidin-1-yl)pyrazin-2-yl)-2-fluorovinyl)-3-phenoxy-2-(trifluoromethyl)phenyl)-2,9-diazaspiro[5.5]undecane;
(Z)-2-(6-(2-(6-(3,3-difluoropyrrolidin-1-yl)pyrazin-2-yl)-2-fluorovinyl)-3-phenoxy-2-(trifluoromethyl)phenyl)-9-ethyl-2,9-diazaspiro[5.5]undecane;
(Z)-9-(cyclopropylmethyl)-2-(6-(2-(6-(3,3-difluoropyrrolidin-1-yl)pyrazin-2-yl)-2-fluorovinyl)-3-(phenoxy-ds)-2-(trifluoromethyl)phenyl)-2,9-diazaspiro[5.5]undecane;
(Z)-2-(6-(2-(6-(3,3-difluoropyrrolidin-1-yl)pyrazin-2-yl)-2-fluorovinyl)-3-(phenoxy-d₅)-2-(trifluoromethyl)phenyl)-9-ethyl-2,9-diazaspiro[5.5]undecane;
(Z)-9-(cyclopropylmethyl)-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane;
(Z)-9-ethyl-4-(6-(2-fluoro-2-(4-(pyridazin-4-yl)pyrimidin-2-yl)vinyl)-3-phenoxy-2-(trifluoromethyl)phenyl)-1-oxa-4,9-diazaspiro[5.5]undecane;
(Z)-4-(6-(2-fluoro-2-(4-(pyridazin-4-yl)pyrimidin-2-yl)vinyl)-3-phenoxy-2-(trifluoromethyl)phenyl)-9-(2-methoxyethyl)-1-oxa-4,9-diazaspiro[5.5]undecane;
(Z)-4-(6-(2-fluoro-2-(6-(pyridazin-4-yl)pyridin-2-yl)vinyl)-3-phenoxy-2-(trifluoromethyl)phenyl)-9-(2-methoxyethyl)-1-oxa-4,9-diazaspiro[5.5]undecane;
(Z)-9-(cyclopropylmethyl)-4-(6-(2-fluoro-2-(6-(pyridazin-4-yl)pyridin-2-yl)vinyl)-3-phenoxy-2-(trifluoromethyl)phenyl)-1-oxa-4,9-diazaspiro[5.5]undecane;
(Z)-9-(cyclopropylmethyl)-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane;
(Z)-9-(cyclopropylmethyl)-2-(6-(2-fluoro-2-(6-(pyridazin-4-yl)pyrazin-2-yl)vinyl)-3-phenoxy-2-(trifluoromethyl)phenyl)-2,9-diazaspiro[5.5]undecane;
(Z)-9-cyclobutyl-2-(6-(2-fluoro-2-(4-(pyridazin-4-yl)pyrimidin-2-yl)vinyl)-3-phenoxy-2-(trifluoromethyl)phenyl)-2,9-diazaspiro[5.5]undecane;
(Z)-2-(6-(2-fluoro-2-(4-(pyridazin-4-yl)pyrimidin-2-yl)vinyl)-3-phenoxy-2-(trifluoromethyl)phenyl)-9-(2-methoxyethyl)-2,9-diazaspiro[5.5]undecane;
(Z)-9-ethyl-2-(6-(2-fluoro-2-(4-(pyridazin-4-yl)pyrimidin-2-yl)vinyl)-3-((3-fluoropyridin-2-yl)oxy)-2-(trifluoromethyl)phenyl)-2,9-diazaspiro[5.5]undecane;
(Z)-9-ethyl-2-(6-(2-fluoro-2-(6-(pyridazin-4-yl)pyridin-2-yl)vinyl)-3-((3-fluoropyridin-2-yl)oxy)-2-(trifluoromethyl)phenyl)-2,9-diazaspiro[5.5]undecane;
(Z)-9-ethyl-4-(6-(2-fluoro-2-(6-(pyridazin-4-yl)pyrazin-2-yl)vinyl)-3-(3-fluorophenoxy)-2-(trifluoromethyl)phenyl)-1-oxa-4,9-diazaspiro[5.5]undecane;
(Z)-9-ethyl-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane;
(Z)-9-(cyclopropylmethyl)-4-(6-(2-fluoro-2-(6-(pyridazin-4-yl)pyrazin-2-yl)vinyl)-3-(2-fluorophenoxy)-2-(trifluoromethyl)phenyl)-1-oxa-4,9-diazaspiro[5.5]undecane;
(Z)-9-(cyclopropylmethyl)-2-(6-(2-fluoro-2-(6-(pyridazin-4-yl)pyrazin-2-yl)vinyl)-3-(2-fluorophenoxy)-2-(trifluoromethyl)phenyl)-2,9-diazaspiro[5.5]undecane;
(Z)-9-(cyclopropylmethyl)-2-(3-(2,4-difluorophenoxy)-6-(2-fluoro-2-(6-(pyridazin-4-yl)pyrazin-2-yl)vinyl)-2-(trifluoromethyl)phenyl)-2,9-diazaspiro[5.5]undecane;
(Z)-9-(cyclopropylmethyl)-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane;
(Z)-9-ethyl-2-(6-(2-fluoro-2-(4-(pyridazin-4-yl)pyrimidin-2-yl)vinyl)-3-(2-fluorophenoxy)-2-(trifluoromethyl)phenyl)-2,9-diazaspiro[5.5]undecane;
(Z)-9-ethyl-4-(6-(2-fluoro-2-(4-(pyridazin-4-yl)pyrimidin-2-yl)vinyl)-3-(2-fluorophenoxy)-2-(trifluoromethyl)phenyl)-1-oxa-4,9-diazaspiro[5.5]undecane;
(Z)-9-(cyclopropylmethyl)-4-(6-(2-fluoro-2-(4-(pyridazin-4-yl)pyrimidin-2-yl)vinyl)-3-(2-fluorophenoxy)-2-(trifluoromethyl)phenyl)-1-oxa-4,9-diazaspiro[5.5]undecane;
(S,Z)-(1-(6-(2-fluoro-2-(4-(pyridazin-4-yl)pyrimidin-2-yl)vinyl)-3-phenoxy-2-(trifluoromethyl)phenyl)piperidin-3-yl)methanamine;
(Z)-2-(6-(2-(6-(3,3-difluoroazetidin-1-yl)pyrazin-2-yl)-2-fluorovinyl)-3-(2-fluorophenoxy)-2-(trifluoromethyl)phenyl)-9-ethyl-2,9-diazaspiro[5.5]undecane;
(Z)-2-(2-(6-(2-fluoro-2-(4-(isothiazol-4-yl)pyrimidin-2-yl)vinyl)-3-(2-fluorophenoxy)-2-(trifluoromethyl)phenyl)-2,9-diazaspiro[5.5]undecan-9-yl)ethan-1-ol;
(Z)-4-(2-(2-(2-(9-ethyl-2,9-diazaspiro[5.5]undecan-2-yl)-4-(2-fluorophenoxy)-3-(trifluoromethyl)phenyl)-1-fluorovinyl)pyrimidin-4-yl)isothiazole;
(Z)-4-(6-(2-(2-(9-(cyclopropylmethyl)-2,9-diazaspiro[5.5]undecan-2-yl)-4-(2,3-difluorophenoxy)-3-(trifluoromethyl)phenyl)-1-fluorovinyl)pyrazin-2-yl)isothiazole;
(Z)-2-(3-(cyclohexyloxy)-6-(2-fluoro-2-(6-(pyridazin-4-yl)pyrazin-2-yl)vinyl)-2-(trifluoromethyl)phenyl)-9-(cyclopropylmethyl)-2,9-diazaspiro[5.5]undecane;
(Z)-5-(6-(2-(2-(9-(cyclopropylmethyl)-2,9-diazaspiro[5.5]undecan-2-yl)-4-(2,3-difluorophenoxy)-3-(trifluoromethyl)phenyl)-1-fluorovinyl)pyrazin-2-yl)isothiazole;
(Z)-2-(3-(cyclohexyloxy)-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane;
(Z)-2-(3-(cyclopentyloxy)-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane;
(Z)-2-(3-cyclobutoxy-6-(2-fluoro-2-(4-(pyridazin-4-yl)pyrimidin-2-yl)vinyl)-2-(trifluoromethyl)phenyl)-9-(cyclopropylmethyl)-2,9-diazaspiro[5.5]undecane;
(Z)-2-(3-(cyclopentyloxy)-6-(2-fluoro-2-(4-(pyridazin-4-yl)pyrimidin-2-yl)vinyl)-2-(trifluoromethyl)phenyl)-9-ethyl-2,9-diazaspiro[5.5]undecane;
(Z)-2-(3-(bicyclo[3.1.1]heptan-3-yloxy)-6-(2-fluoro-2-(4-(pyridazin-4-yl)pyrimidin-2-yl)vinyl)-2-(trifluoromethyl)phenyl)-9-ethyl-2,9-diazaspiro[5.5]undecane;
(Z)-4-(2-(2-(2-(9-(cyclopropylmethyl)-2,9-diazaspiro[5.5]undecan-2-yl)-4-(2-fluorophenoxy)-3-(trifluoromethyl)phenyl)-1-fluorovinyl)pyrimidin-4-yl)isothiazole;
(Z)-4-(6-(2-(2-(9-(cyclopropylmethyl)-2,9-diazaspiro[5.5]undecan-2-yl)-4-(2-fluorophenoxy)-3-(trifluoromethyl)phenyl)-1-fluorovinyl)pyrazin-2-yl)isothiazole;
(Z)-9-(cyclopropylmethyl)-2-(6-(2-(6-(3,3-difluoroazetidin-1-yl)pyrazin-2-yl)-2-fluorovinyl)-3-(2-fluorophenoxy)-2-(trifluoromethyl)phenyl)-2,9-diazaspiro[5.5]undecane;
(Z)-2-(6-(2-(6-(3,3-difluoropyrrolidin-1-yl)pyrazin-2-yl)-2-fluorovinyl)-3-(2-fluorophenoxy)-2-(trifluoromethyl)phenyl)-9-ethyl-2,9-diazaspiro[5.5]undecane; and
(Z)-9-(cyclopropylmethyl)-2-(6-(2-(6-(3,3-difluoropyrrolidin-1-yl)pyrazin-2-yl)-2-fluorovinyl)-3-(2-fluorophenoxy)-2-(trifluoromethyl)phenyl)-2,9-diazaspiro[5.5]undecane;
or a pharmaceutically acceptable salt thereof.

61. The compound of claim 1, or a pharmaceutically acceptable salt thereof, wherein the compound is deuterated.

62. A pharmaceutical composition, comprising a compound of claim 1, or a pharmaceutically acceptable salt thereof, and a pharmaceutically acceptable carrier.

63. A method of inhibiting an activity of a diacylglycerol kinase, comprising contacting the kinase with a compound of claim 1, or a pharmaceutically acceptable salt thereof.

64. A method of treating cancer in a patient in need thereof, the method comprising administering to the patient a therapeutically effective amount of a compound of claim 1, or a pharmaceutically acceptable salt thereof.

65. The method of claim 64, wherein the cancer is non-small cell lung cancer, bladder urothelial carcinoma, esophageal carcinoma, stomach adenocarcinoma, mesothelioma, liver hepatocellular carcinoma, diffuse large B cell lymphoma, kidney renal clear cell carcinoma, head and neck squamous cell carcinoma, cholangiocarcinoma, cervical squamous cell carcinoma, endocervical adenocarcinoma, and melanoma.

66. The method of claim 65, wherein the melanoma is metastatic melanoma.
